(19) United States
(12) Patent Application Publication
(10) Pub. No.: US 2026/0146100 A1
(43) Pub. Date: May 28, 2026

(54) MULTISPECIFIC ANTIGEN-BINDING MOLECULES THAT BIND CD22 AND 4-1BB AND METHODS OF USE THEREOF

(71) Applicant: Regeneron Pharmaceuticals, Inc., Tarrytown, NY (US)

(72) Inventors: Joyce Wei, New York, NY (US); Kara L. Olson, White Plains, NY (US); Erica Ullman, Yorktown Heights, NY (US)

(21) Appl. No.: 19/372,768

(22) Filed: Oct. 29, 2025

Related U.S. Application Data
(60) Provisional application No. 63/713,829, filed on Oct. 30, 2024.

(51) Int. Cl. C07K 16/28; A61K 39/00; A61K 45/06; A61P 35/00

(52) U.S. Cl. CPC C07K 16/2878; A61K 45/06; A61P 35/00; C07K 16/2803; A61K 2039/505; C07K 2317/14; C07K 2317/31; C07K 2317/522; C07K 2317/524; C07K 2317/526; C07K 2317/53; C07K 2317/565

(57) ABSTRACT

The present disclosure provides multispecific anti-CD22/anti-4-1BB antigen-binding molecules comprising a first antigen-binding domain that binds specifically to CD22 and a second antigen-binding domain that binds specifically to 4-1BB. In certain embodiments, the molecules further comprise a third antigen-binding domain that binds 4-1BB. In certain embodiments, the molecules are multispecific antibodies or antigen-binding fragments thereof. In certain embodiments, the antibodies are useful in treating a CD22-associated disease or disorder (e.g., lymphoma).

### CROSS-REFERENCE TO RELATED APPLICATION

This application claims the benefit of priority to U.S. Provisional Patent Appl. No. 63/713,829 filed Oct. 30, 2024, the disclosure of which is hereby incorporated by reference herein in its entirety.

### FIELD

The present disclosure relates to multispecific antigen-binding molecules that bind CD22 and 4-1BB and methods of use thereof, e.g., for treating or preventing cancer.

### SEQUENCE LISTING

The sequence listing of the present application is submitted electronically as an ST.26 formatted xml file with a file name “SeqList11731.xml,” creation date of Oct. 24, 2025, and a size of 63,385 bytes.

### BACKGROUND

The ability of T cells to recognize and kill their cellular targets, such as virally-infected cells or tumor cells, depends on a coordinated set of interactions. Two signals, “signal 1” & “signal 2”, are required for proper T cell activation. Signal 1 involves recognition and binding of the target cell by the T cell Receptor (TCR) complex (which includes the associated CD3 gamma (γ), delta (δ), epsilon (ε), and zeta (ζ) chains). The TCR recognizes a viral or tumor peptide presented on the groove of an MHC protein expressed on the surface of a target cell. Because such binding is generally of low-affinity, successful triggering of signal 1 requires clustering of many TCR complexes along the interface between the T cell and its target cell. This interface has been referred to as the “immune synapse.” Signal 2 is provided by engaging co-stimulatory receptors on T cells. One such costimulatory receptor is 4-1BB, which is an inducible type I membrane protein and member of the tumor necrosis factor receptor (TNFR) superfamily. Expression of 4-1BB receptor is induced on the surface of T cells after antigen- or mitogen-induced activation. When a T cell recognizes its target cell via its TCR complex, and then also engages signal 2 via 4-1BB binding to its cognate ligand(s) on the target cell, T cell activation is enhanced.

4-1BB has been garnering attention as a promising therapeutic target in the setting of cancer, amongst other diseases, due to its broad expression profile and ability to stimulate various signaling pathways involved in the generation of a potent immune response. As a prominent mediator of immune responses expressed on various cell types, 4-1BB signaling not only exerts protective effects, but is also capable of driving pathologies such as the adverse effects observed following administration of therapeutic 4-1BB antibodies. The persistent stimulation of 4-1BB signaling and, consequently, continuous activation of T cells have been shown to result in granuloma formation in tumor-draining lymph nodes due to the excessive recruitment of macrophages (Kim et al. Cell Mol. Immunol. (2021) 18(8):1956-68). Furthermore, liver-associated toxicity has been reported to be a common problem associated with therapeutic 4-1BB antibody treatment. Urelumab (BMS-663513), a fully human IgG4 monoclonal antibody, was the first anti-4-1BB therapeutic to enter clinical trials. Clinical development halted when liver toxicity associated with the antibody was revealed. It has been showed that 4-1BB antibody treatment results in CD8⁺ T cell infiltration into the liver causing inflammation and increased transaminase expression (Dubrot et al. Cancer Immunol. Immunother. (2010) 59(8):1223-33). Such infiltration, however, was not associated with clinical benefit in the setting of tumors in or around the liver tissue. Localized or targeted use of anti-4-1BB mAb can be used for promotion of antitumor immunity with less risk.

Cluster of Differentiation-22 (CD22; also known as Siglec-2), a member of the Siglec family, specifically recognizes α2, 6 sialic acid and is a transmembrane protein preferentially expressed on B lymphocytes (B cells). CD22 has a number of ascribed functions including, for example, B cell homeostasis, B cell survival and migration, dampening TLR and CD40 signaling, and inhibiting B cell receptor (BCR) signaling via recruitment of SH2 domain-containing phosphatases by phosphorylation of immunoreceptor tyrosine-based inhibition motifs (ITIMs) in the cytoplasmic region, as well as facilitation of adhesion between B cells and other cell types. CD22 is not found on the surface of B cells during the early stages of development, nor is it expressed in stem cells. However, 60-70% of all B-cell lymphomas and leukemias express CD22. An anti-CD22 antibody for treating B-cell lymphomas and leukemias has been investigated. However, the monoclonal antibody, Epratuzumab, had limited success (Grant, et al. (2013) Cancer 119(21):3797-804). Thus, a need exists in the art for alternative approaches to treating cancer. Multispecific antigen-binding molecules that bind both CD22 and 4-1BB would be useful in therapeutic settings in which specific targeting and T cell-mediated killing of tumor cells that express CD22 is desired. There is a need for improved immunotherapeutic agents that recognize CD22 and 4-1BB.

### SUMMARY

The present disclosure provides antigen-binding molecules that bind both CD22 and 4-1BB (“anti-CD22×anti-4-1BB”). The multispecific anti-CD22×anti-4-1BB antigen-binding molecules of the present disclosure bind to and engage CD22 expressing tumor cells while also targeting 4-1BB. CD22 is expressed on malignant B cells. As such, the anti-CD22×anti-4-1BB antigen-binding molecules disclosed herein provide an efficacious anti-tumor therapy against B cell lymphomas and leukemias.

In one aspect, the disclosed technology relates to an isolated multispecific antigen-binding molecule comprising: (a) a first antigen-binding domain (D1) containing three complementarity determining regions (CDRs) (D1-HCDR1, D1-HCDR2, and D1-HCDR3) of a heavy chain variable region (D1-HCVR) and three CDRs (D1-LCDR1, D1-LCDR2, and D1-LCDR3) of a light chain variable region (D1-LCVR), wherein the first antigen-binding domain binds specifically to CD22; (b) a second antigen-binding domain (D2) containing three CDRs (D2-HCDR1, D2-HCDR2, and D2-HCDR3) of a HCVR (D2-HCVR) and three CDRs (D2-LCDR1, D2-LCDR2, and D2-LCDR3) of a LCVR (D2-LCVR), wherein the second antigen-binding domain binds specifically to 4-1BB; and (c) a third antigen-binding domain (D3) containing three CDRs (D3-HCDR1, D3-HCDR2, and D3-HCDR3) of a HCVR (D3-HCVR) and three CDRs (D3-LCDR1, D3-LCDR2, and D3-LCDR3) of a LCVR (D3-LCVR), wherein the third antigen-binding domain binds specifically to 4-1BB.

In some embodiments, D2 and D3 bind to the same epitope on 4-1BB. In some embodiments, D2 and D3 bind to different epitopes on 4-1BB. In some embodiments, D1 comprises three CDRs of a HCVR containing an amino acid sequence of SEQ ID NO: 2 or 32. In some embodiments, D1 comprises three CDRs of a LCVR containing an amino acid sequence of SEQ ID NO: 18. In some embodiments, D1-HCDR1, D1-HCDR2, and D1-HCDR3 comprise respective amino acid sequences of SEQ ID NO: 4, SEQ ID NO: 6, and SEQ ID NO: 8; or SEQ ID NO: 34, SEQ ID NO: 36, and SEQ ID NO: 38. In some embodiments, D1-LCDR1, D1-LCDR2, and D1-LCDR3 comprise respective amino acid sequences of SEQ ID NO: 20, GAS, and SEQ ID NO: 24. In some embodiments, D1-HCVR comprises an amino acid sequence of SEQ ID NO: 2 or 32. In some embodiments, D1-LCVR comprises an amino acid sequence of SEQ ID NO: 18.

In some embodiments, D2 comprises three CDRs of a HCVR comprising an amino acid sequence of SEQ ID NO: 10 or 42. In some embodiments, D2 comprises three CDRs of a LCVR comprising an amino acid sequence of SEQ ID NO: 18. In some embodiments, D2-HCDR1, D2-HCDR2, and D2-HCDR3 comprise respective amino acid sequences of SEQ ID NO: 12, SEQ ID NO: 14, and SEQ ID NO: 16; or SEQ ID NO: 44, SEQ ID NO: 46, and SEQ ID NO: 48. In some embodiments, D2-LCDR1, D2-LCDR2, and D2-LCDR3 comprise respective amino acid sequences of SEQ ID NO: 20, GAS, and SEQ ID NO: 24. In some embodiments, D2-HCVR comprises an amino acid sequence of SEQ ID NO: 10 or 42. In some embodiments, D2-LCVR comprises an amino acid sequence of SEQ ID NO: 18.

In some embodiments, D3 comprises three CDRs of a HCVR comprising an amino acid sequence of SEQ ID NO: 10 or 42. In some embodiments, D3 comprises three CDRs of a LCVR comprising an amino acid sequence of SEQ ID NO: 18. In some embodiments, D3-HCDR1, D3-HCDR2, and D3-HCDR3 comprise respective amino acid sequences of SEQ ID NO: 12, SEQ ID NO: 14, and SEQ ID NO: 16; or SEQ ID NO: 44, SEQ ID NO: 46, and SEQ ID NO: 48. In some embodiments, D3-LCDR1, D3-LCDR2, and D3-LCDR3 comprise respective amino acid sequences of SEQ ID NO: 20, GAS, and SEQ ID NO: 24. In some embodiments, D3-HCVR comprises the amino acid sequence of SEQ ID NO: 10 or 42. In some embodiments, D3-LCVR comprises the amino acid sequence of SEQ ID NO: 18. In some embodiments, D1-LCVR, D2-LCVR, and D3-LCVR comprise the same amino acid sequence.

In some embodiments, D1 comprises three CDRs of a HCVR comprising an amino acid sequence of SEQ ID NO: 2 or 32; D2 comprises three CDRs of a HCVR comprising an amino acid sequence of SEQ ID NO: 10 or 42; and D3 comprises three CDRs of a HCVR comprising an amino acid sequence of SEQ ID NO: 10 or 42. In some embodiments, D1 comprises three CDRs of a HCVR comprising an amino acid sequence of SEQ ID NO: 2; D2 comprises three CDRs of a HCVR comprising an amino acid sequence of SEQ ID NO: 10; and D3 comprises three CDRs of a HCVR comprising an amino acid sequence of SEQ ID NO: 10. In some embodiments, D1 comprises three CDRs of a HCVR comprising an amino acid sequence of SEQ ID NO: 2; D2 comprises three CDRs of a HCVR comprising an amino acid sequence of SEQ ID NO: 42; and D3 comprises three CDRs of a HCVR comprising an amino acid sequence of SEQ ID NO: 42. In some embodiments, D1 comprises three CDRs of a HCVR comprising an amino acid sequence of SEQ ID NO: 32; D2 comprises three CDRs of a HCVR comprising an amino acid sequence of SEQ ID NO: 10; and D3 comprises three CDRs of a HCVR comprising an amino acid sequence of SEQ ID NO: 10. In some embodiments, D1-HCVR, D2-HCVR, and D3-HCVR comprise respective amino acid sequences of SEQ ID NOs: 2, 10, and 10. In some embodiments, D1-HCVR, D2-HCVR, and D3-HCVR comprise respective amino acid sequences of SEQ ID NOs: 2, 42, and 42. In some embodiments, D1-HCVR, D2-HCVR, and D3-HCVR comprise respective amino acid sequences of SEQ ID NOs: 32, 10, and 10.

In some embodiments, D1-HCVR, D2-HCVR, and D3-HCVR comprise respective amino acid sequences of SEQ ID NOs: 2, 10, and 10; and D1-LCVR, D2-LCVR, and D3-LCVR comprise respective amino acid sequences of SEQ ID NOs: 18, 18, and 18. In some embodiments, D1-HCVR, D2-HCVR, and D3-HCVR comprise respective amino acid sequences of SEQ ID NOs: 2, 42, and 42; and D1-LCVR, D2-LCVR, and D3-LCVR comprise respective amino acid sequences of SEQ ID NOs: 18, 18, and 18. In some embodiments, D1-HCVR, D2-HCVR, and D3-HCVR comprise respective amino acid sequences of SEQ ID NOs: 32, 10, and 10; and D1-LCVR, D2-LCVR, and D3-LCVR comprise respective amino acid sequences of SEQ ID NOs: 18, 18, and 18.

In some embodiments, the multispecific antigen-binding protein is a multispecific antibody or antigen-binding fragment thereof. In some embodiments, the multispecific antigen-binding the molecule is a multispecific antibody comprising a first heavy chain comprising a HCVR of a first antigen-binding arm, which binds CD22, wherein the first heavy chain is paired with a first light chain comprising a LCVR of the first antigen-binding arm, wherein (i) the first antigen-binding arm binds CD22, (ii) the HCVR is D1-HCVR, and (iii) the LCVR is D1-LCVR. In some embodiments, the multispecific antigen-binding the molecule is a multispecific antibody further comprising a second heavy chain comprising an outer HCVR and an inner HCVR of a second antigen-binding arm, wherein the second heavy chain is paired with an outer light chain and an inner light chain comprising an outer LCVR and an inner LCVR, respectively, of the second antigen-binding arm, wherein (i) the second antigen-binding arm binds 4-1BB, (ii) the outer HCVR is D2-HCVR, (iii) the inner HCVR is D3-HCVR, (iv) the outer LCVR is D2-LCVR, and (vi) the inner LCVR is D3-LCVR.

In some embodiments, the first heavy chain and the first light chain are interconnected by disulfide bonds, wherein the first heavy chain comprises D1-HCVR and a heavy chain constant region comprising CH1, CH2, and CH3 domains, and the first light chain comprises D1-LCVR and a light chain constant region, wherein the first heavy chain and first light chain comprise the first antigen-binding domain; and the second heavy chain and the outer light chain are interconnected by disulfide bonds, and the second heavy chain and the inner light chain are interconnected by disulfide bonds, wherein the second heavy chain comprises D2-HCVR and D3-HCVR and a heavy chain constant region comprising CH1, CH2, and CH3 domains, and the outer light chain comprises D2-LCVR and a light chain constant region, and the inner light chain comprises D3-LCVR and a light chain constant region, wherein the second heavy chain and the outer light chain comprise the second antigen-binding domain, and the second heavy chain and the inner light chain comprise the third antigen-binding domain. In some embodiments, the first heavy chain or the second heavy chain, but not both, comprises a CH3 domain comprising a H435R (EU numbering) modification and a Y436F (EU numbering) modification. In some embodiments, the heavy chain constant region of the first heavy chain and/or the heavy chain constant region of the second heavy chain are of isotype IgG1. In some embodiments, the heavy chain constant region of the first heavy chain and/or the heavy chain constant region of the second heavy chain are of isotype IgG4. In some embodiments, the first heavy chain and the second heavy chain comprise a chimeric hinge that reduces Fcγ receptor binding relative to a wild-type hinge of the same isotype.

In some embodiments, the first heavy chain comprises an amino acid sequence of SEQ ID NO: 26 or 40. In some embodiments, the second heavy chain comprises an amino acid sequence of SEQ ID NO: 28 or 50. In some embodiments, the first heavy chain comprises an amino acid sequence of SEQ ID NO: 26, and the second heavy chain comprises an amino acid sequence of SEQ ID NO: 28. In some embodiments, the first heavy chain comprises an amino acid sequence of SEQ ID NO: 26, and the second heavy chain comprises an amino acid sequence of SEQ ID NO: 50. In some embodiments, the first heavy chain comprises an amino acid sequence of SEQ ID NO: 40, and the second heavy chain comprises an amino acid sequence of SEQ ID NO: 28. In some embodiments, the first light chain, the outer light chain, and the inner light chain comprise a common light chain. In some embodiments, the common light chain comprises an amino acid sequence of SEQ ID NO: 30.

In one aspect, the disclosed technology relates to a pharmaceutical composition comprising an isolated multispecific antigen-binding molecule that binds specifically to CD22 and 4-1BB, and a pharmaceutically acceptable carrier or diluent.

In one aspect, the disclosed technology relates to a method of producing a multispecific antigen-binding molecule that binds specifically to CD22 and 4-1BB, the method comprising introducing one or more nucleic acid molecules comprising polynucleotide sequences that encode the antigen-binding molecule into a host cell, and culturing the host cell under conditions favorable to expression of the one or more nucleic acid molecules. In some embodiments, the method further comprises isolating the multispecific antigen-binding molecule from the host cell and/or medium in which the host cell is grown. In some embodiments, the host cell is a Chinese hamster ovary (CHO) cell.

In one aspect, the disclosed technology relates to an isolated nucleic acid molecule or a group of nucleic acid molecules. In some embodiments, the nucleic acid molecule or group of nucleic acid molecules comprise a polynucleotide sequence encoding an amino acid sequence comprising D1-HCVR, D2-HCVR, D3-HCVR, D1-LCVR, D2-LCVR, and D3-LCVR of an multispecific antigen-binding molecule that binds specifically to CD22 and 4-1BB. In some embodiments, an expression vector contains a nucleic acid molecule or group of nucleic acid molecules comprising a polynucleotide sequence encoding an amino acid sequence comprising D1-HCVR, D2-HCVR, D3-HCVR, D1-LCVR, D2-LCVR, and D3-LCVR of an multispecific antigen-binding molecule that binds specifically to CD22 and 4-1BB. In one embodiment the expression vector or set of expression vectors is contained within a host cell. In one embodiment the host cell is E. coli or CHO cell.

In one aspect, the disclosed technology relates to a host cell comprising: a first expression vector comprising a nucleic acid molecule encoding a first immunoglobulin heavy chain comprising the amino acid sequence of SEQ ID NO: 26; a second expression vector comprising a nucleic acid molecule encoding a second immunoglobulin heavy chain comprising the amino acid sequence of SEQ ID NO: 28; and a third expression vector comprising a nucleic acid molecule encoding an immunoglobulin light chain comprising the amino acid sequence of SEQ ID NO: 30.

In one aspect, the disclosed technology relates to a host cell comprising: a first expression vector comprising a nucleic acid molecule encoding a first immunoglobulin heavy chain comprising the amino acid sequence of SEQ ID NO: 26; a second expression vector comprising a nucleic acid molecule encoding a second immunoglobulin heavy chain comprising the amino acid sequence of SEQ ID NO: 50; and a third expression vector comprising a nucleic acid molecule encoding an immunoglobulin light chain comprising the amino acid sequence of SEQ ID NO: 30.

In one aspect, the disclosed technology relates to a host cell comprising: a first expression vector comprising a nucleic acid molecule encoding a first immunoglobulin heavy chain comprising the amino acid sequence of SEQ ID NO: 40; a second expression vector comprising a nucleic acid molecule encoding a second immunoglobulin heavy chain comprising the amino acid sequence of SEQ ID NO: 28; and a third expression vector comprising a nucleic acid molecule encoding an immunoglobulin light chain comprising the amino acid sequence of SEQ ID NO: 30.

In one aspect, the disclosed technology relates to a method of producing a multispecific antigen-binding molecule that specifically binds CD22 and 4-1BB comprising: (a) culturing a host cell under conditions favorable for production of the multispecific antigen-binding molecule; and (b) optionally, isolating the multispecific antigen-binding molecule from the host cell and/or medium in which the host cell is grown. In some embodiments, the host cell is a CHO cell. In some embodiments, the method further comprises formulating the multispecific antigen-binding molecule as a pharmaceutical composition comprising an acceptable carrier.

In one aspect, the disclosed technology relates to a method of inhibiting growth of a tumor in a subject, comprising administering the isolated multispecific antigen-binding molecule that specifically binds CD22 and 4-1BB. In some embodiments, the tumor is anal cancer, angiosarcoma, basal cell carcinoma, a B cell cancer, bladder cancer, bone cancer, brain cancer, breast cancer, cervical cancer, cholangiocarcinoma, chondrosarcoma, colon cancer, colorectal cancer, cutaneous squamous cell carcinoma, endometrial cancer, esophageal cancer, glioblastoma multiforme, head and neck squamous cell cancer, hepatocellular carcinoma, kidney cancer, liver cancer, leukemia, lung cancer, lymphoma, Merkel cell carcinoma, melanoma, myeloma, non-small cell lung cancer, ovarian cancer, pancreatic cancer, prostate cancer, salivary gland cancer, skin cancer, soft tissue sarcoma, stomach cancer, a T cell cancer, testicular cancer, and uterine cancer. In some embodiments, the method further comprises administering a second therapeutic agent or therapeutic regimen. In some embodiments, the second therapeutic agent or therapeutic regimen comprises an Ang2 inhibitor, a BCMA inhibitor, a bispecific antibody comprising a CD28-binding arm, an anti-CD20×anti-CD3 multispecific antigen-binding molecule, an antibody drug conjugate, a bispecific antibody conjugated to an anti-tumor agent, a cancer vaccine, a CD20 inhibitor, a CD19 inhibitor, a CD27 agonist, a CD28 agonist, CD38 inhibitor, a chemotherapeutic drug, a checkpoint inhibitor, a corticosteroid, a CTLA-4 inhibitor, a cytokine, a DNA alkylator, an EGFR inhibitor, a GITR agonist, a histone deacetylase inhibitor, an IL4 inhibitor, an IL6 inhibitor, an immunocytokine, an immunomodulator, a LAG3 inhibitor, a modified IL2, a modified IL12, a MUC16 inhibitor, an oncolytic virus, a PD-1 inhibitor, a PD-L1 inhibitor, a proteasome inhibitor, radiotherapy, a stem cell transplant, surgery, a T cell comprising a chimeric antigen receptor (CAR-Tcell), a VEGF inhibitor, a 4-1BB activator, or combinations thereof.

In one aspect, the disclosed technology relates to a kit comprising an isolated multispecific antigen-binding molecule that specifically binds CD22 and 4-1BB in combination with written instructions for use of a therapeutically effective amount of the isolated multispecific antigen-binding molecule for inhibiting the growth of a tumor in a subject.

### BRIEF DESCRIPTION OF THE FIGURES

FIG. 1 relates to Example 3 and is a graph showing binding of the indicated antibodies to Jurkat/NFκB-Luc/4-1BB cells over the indicated range of concentrations. Comparator 1 and control antibodies are described in Example 1.

FIG. 2 relates to Example 3 and is a graph showing binding of the indicated antibodies to HEK293/hCD20 cells over the indicated range of concentrations. Comparator 1 and control antibodies are described in Example 1.

FIG. 3 relates to Example 3 and is a graph showing binding of the indicated antibodies to HEK293/hCD19/hCD20/hCD22 cells over the indicated range of concentrations. Comparator 1 and control antibodies are described in Example 1.

FIG. 4 relates to Example 3 and is a graph showing binding of the indicated antibodies to Ramos cells over the indicated range of concentrations. Comparator 1 and control antibodies are described in Example 1.

FIG. 5 relates to Example 5 and is a graph showing the concentration of IL-2 produced by human peripheral blood mononuclear cells (PBMCs) in the presence of HEK293/CD20/CD22 cells upon exposure to the indicated antibodies at the indicated range of concentrations. Comparator 1 and control antibodies are described in Example 1.

FIG. 6 relates to Example 5 and is a graph showing the concentration of IFN-γ produced by hPBMCs in the presence of HEK293/CD20/CD22 cells upon exposure to the indicated antibodies at the indicated range of concentrations. Comparator 1 and control antibodies are described in Example 1.

FIG. 7 relates to Example 5 and is a graph showing the concentration of TNF-α produced by hPBMCs in the presence of HEK293/CD20/CD22 cells upon exposure to the indicated antibodies at the indicated range of concentrations. Comparator 1 and control antibodies are described in Example 1.

FIG. 8 relates to Example 5 and is a graph showing the concentration of GM-CSF produced by hPBMCs in the presence of HEK293/CD20/CD22 cells upon exposure to the indicated antibodies at the indicated range of concentrations. Comparator 1 and control antibodies are described in Example 1.

FIG. 9 relates to Example 5 and is a graph showing the concentration of IL-2 produced by hPBMCs in the presence of HEK293/CD20 cells upon exposure to the indicated antibodies at the indicated range of concentrations. Comparator 1 and control antibodies are described in Example 1.

FIG. 10 relates to Example 5 and is a graph showing the concentration of IFN-γ produced by hPBMCs in the presence of HEK293/CD20 cells upon exposure to the indicated antibodies at the indicated range of concentrations. Comparator 1 and control antibodies are described in Example 1.

FIG. 11 relates to Example 5 and is a graph showing the concentration of TNF-α produced by hPBMCs in the presence of HEK293/CD20 cells upon exposure to the indicated antibodies at the indicated range of concentrations. Comparator 1 and control antibodies are described in Example 1.

FIG. 12 relates to Example 5 and is a graph showing the concentration of GM-CSF produced by hPBMCs in the presence of HEK293/CD20 cells upon exposure to the indicated antibodies at the indicated range of concentrations. Comparator 1 and control antibodies are described in Example 1.

FIGS. 13A-13B relate to Example 5. FIG. 13A is a graph showing the concentration of IL-2 produced by hPBMCs in the presence of WSU-DLCL2 cells and 0.2 nM odronextamab upon exposure to the indicated antibodies at the indicated range of concentrations. FIG. 13B is another graph showing the concentration of IL-2 produced by hPBMCs in the presence of WSU-DLCL2 cells and 0.2 nM odronextamab upon exposure to the indicated antibodies at the indicated range of concentrations. Comparator 1 and control antibodies are described in Example 1.

FIG. 14 relates to Example 5 and is a graph showing the concentration of IFN-γ produced by hPBMCs in the presence of WSU-DLCL2 cells and 0.2 nM odronextamab upon exposure to the indicated antibodies at the indicated range of concentrations. Comparator 1 and control antibodies are described in Example 1.

FIG. 15 relates to Example 5 and is a graph showing the concentration of TNF-α produced by hPBMCs in the presence of WSU-DLCL2 cells and 0.2 nM odronextamab upon exposure to the indicated antibodies at the indicated range of concentrations. Comparator 1 and control antibodies are described in Example 1.

FIG. 16 relates to Example 5 and is a graph showing the concentration of GM-CSF produced by hPBMCs in the presence of WSU-DLCL2 cells and 0.2 nM odronextamab upon exposure to the indicated antibodies at the indicated range of concentrations. Comparator 1 and control antibodies are described in Example 1.

FIG. 17 relates to Example 6 and is a graph showing the killing of Ramos/GFP cells over time according to area of the plate with GFP signal upon addition of 200 pM odronextamab or odronextamab in combination with the indicated concentrations of REGN9220.

FIG. 18 relates to Example 6 and is a graph showing the killing of Ramos/GFP cells over time according to area of the plate with GFP signal upon addition of 200 pM odronextamab or odronextamab in combination with the indicated concentrations of the CD22×CD28 bispecific antibody antibody REGN5837.

FIG. 19 relates to Example 6 and is a graph showing the percent survival of Ramos/GFP cells 192 hours after addition of REGN9220 or the CD22×CD28 bispecific antibody REGN5837 at the indicated range of concentrations. Statistical significance calculated using the Kaplan-Meier method with log-rank test. *P<0.05, **P<0.01, ***P<0.001.

FIG. 20 relates to Example 7 and is a graph showing average tumor volume over time in mice administered the indicated antibodies at the indicated doses. Statistical significance calculated with 2-way ANOVA and Tukey's multiple comparisons. *P<0.05, **P<0.01, ***P<0.001.

FIG. 21 relates to Example 7 and is a graph showing the probability of survival at the indicated days post implantation of mice administered the indicated antibodies at the indicated doses. Statistical significance calculated using the Kaplan-Meier method with log-rank test. *P<0.05, **P<0.01, ***P<0.001.

FIG. 22 relates to Example 7 and is a set of graphs showing tumor volume over time in individual mice administered the indicated antibodies at the indicated doses. The fraction of tumor-free mice is indicated.

FIG. 23 relates to Example 7 and is a set of graphs showing tumor volume over time in individual mice administered the indicated antibodies at the indicated doses. The fraction of tumor-free mice is indicated.

FIG. 24 relates to Example 8 and is a graph showing average tumor volume over time in mice administered the indicated antibodies at the indicated doses. Statistical significance calculated with 2-way ANOVA and Tukey's multiple comparisons. *P<0.05, **P<0.01, ***P<0.001.

FIG. 25 relates to Example 8 and is a graph showing the probability of survival at the indicated days post implantation of mice administered the indicated antibodies at the indicated doses. Statistical significance calculated using the Kaplan-Meier method with log-rank test. *P<0.05, **P<0.01, ***P<0.001, ****P<0.0001.

FIG. 26 relates to Example 8 and is a set of graphs showing blood cytokine concentration of the indicated cytokines in mice administered the indicated antibodies at the indicated doses.

FIG. 27 relates to Example 9 and is a set of graphs showing tumor volume over time in individual mice administered the indicated antibodies at the indicated doses. The fraction of tumor-free mice is indicated.

FIG. 28 relates to Example 9 and is a set of graphs showing tumor volume over time in individual mice administered the indicated antibodies at the indicated doses. The fraction of tumor-free mice is indicated.

FIG. 29 relates to Example 9 and is a graph showing the probability of survival at the indicated days post implantation of mice administered the indicated antibodies at the indicated doses. Statistical significance calculated using the Kaplan-Meier method with log-rank test. *P<0.05, **P<0.01 ***P<0.001.

FIG. 30 relates to Example 9 and is a set of graphs showing blood cytokine concentration of the indicated cytokines in mice administered the indicated antibodies at the indicated doses.

FIG. 31 relates to Example 10 and is a diagram illustrating the experimental design of the in vivo study.

FIG. 32 relates to Example 10 and is a graph showing tumor weight in individual mice administered the indicated antibodies.

FIG. 33 relates to Example 10 and is a graph showing the percent of live CD45+ cells that are each of the indicated cell types in tumors harvested from mice administered the indicated antibodies.

FIG. 34 relates to Example 10 and is a set of graphs showing the number of the indicated cells per mg of tumor of tumors isolated from individual mice administered the indicated antibodies.

FIG. 35 relates to Example 10 and is a set of graphs showing the number of the indicated cells per μl of blood of individual mice administered the indicated antibodies.

FIG. 36 relates to Example 11 and is a set of graphs showing tumor volume over time in individual mice administered the indicated antibodies at the indicated doses. The fraction of tumor-free mice is indicated.

FIG. 37 relates to Example 11 and is a set of graphs showing tumor volume over time in individual mice administered the indicated antibodies at the indicated doses. The fraction of tumor-free mice is indicated.

FIG. 38 relates to Example 11 and is a graph showing the probability of survival at the indicated days post implantation of mice administered the indicated antibodies at the indicated doses. Statistical significance calculated using the Kaplan-Meier method with log-rank test. *P<0.05, **P<0.01 ***P<0.001.

### DETAILED DESCRIPTION

Before the present disclosure is described, it is to be understood that this disclosure is not limited to particular methods and experimental conditions described, as such methods and conditions may vary. It is also to be understood that the terminology used herein is for the purpose of describing particular embodiments only, and is not intended to be limiting, since the scope of the present disclosure will be limited only by the appended claims.

Unless defined otherwise, all technical and scientific terms used herein have the same meaning as commonly understood by one of ordinary skill in the art to which this disclosure belongs. Although any methods and materials similar or equivalent to those described herein can be used in the practice or testing of the present disclosure, the preferred methods and materials are now described.

The present disclosure relates, in part, to multispecific antigen-binding molecules that specifically bind CD22 (also known as Siglec-2) and 4-1BB (also known as CD137 and TNFRSF9) and their use in treating various diseases, including cancer. The multispecific antigen-binding molecules disclosed herein can be used alone or in combination with other agents for treating cancers that express CD22.

As used herein, “CD22” refers to the human CD22 protein unless specified as being from a non-human species.

As used herein, “4-1BB” refers to the human 4-1BB protein which is expressed on T cells as a costimulatory receptor unless specified as being from a non-human species.

As used herein, “isolated” antigen-binding molecules (e.g., antibodies or antigen-binding fragments thereof), polypeptides, polynucleotides and vectors, refer to such molecules that are at least partially free of other biological molecules from the cells or cell culture from which they are produced. Such biological molecules include nucleic acids, proteins, other antibodies or antigen-binding fragments, lipids, carbohydrates, or other material such as cellular debris and growth medium. An isolated antigen-binding protein may further be at least partially free of expression system components such as biological molecules from a host cell or of the growth medium thereof. The term “isolated” is not intended to refer to a complete absence of such biological molecules or to an absence of water, buffers, or salts or to components of a pharmaceutical formulation that includes the antibodies or antigen-binding fragments thereof.

As used herein, the expression “antigen-binding molecule” means a protein, polypeptide, antibody, fragment of an antibody, or other molecular complex comprising or consisting of at least one complementarity determining region (CDR) that alone, or in combination with one or more additional CDRs and/or framework regions (FRs), specifically binds to a particular antigen. The term includes any naturally occurring, enzymatically obtainable, synthetic, or genetically engineered polypeptide or glycoprotein that specifically binds an antigen to form a complex. In certain embodiments, an antigen-binding molecule is an antibody or a fragment of an antibody.

In some embodiments, an antigen-binding fragment of an antibody comprises at least one variable domain. The variable domain may be of any size or amino acid composition and generally comprises at least one CDR, which is adjacent to or in frame with one or more framework sequences. In antigen-binding fragments having a V_{H} domain associated with a V_{L} domain, the V_{H} and V_{L} domains may be situated relative to one another in any suitable arrangement. For example, the variable region may be dimeric and contain V_{H}-V_{H}, V_{H}-V_{L} or V_{L}-V_{L} dimers. Alternatively, the antigen-binding fragment of an antibody may contain a monomeric V_{H} or V_{L} domain.

In some embodiments, an antigen-binding fragment of an antibody may contain at least one variable domain covalently linked to at least one constant domain. Non-limiting, exemplary configurations of variable and constant domains that may be found within an antigen-binding fragment of an antibody of the present disclosure include: (i) V_{H}-C_{H}1; (ii) V_{H}-C_{H}2; (iii) V_{H}-C_{H}3; (iv) V_{H}-C_{H}1-C_{H}2; (v) V_{H}-C_{H}1-C_{H}2-C_{H}3; (vi) V_{H}-C_{H}2-C_{H}3; (vii) V_{H}-CL; (viii) V_{L}-C_{H}1; (ix) V_{L}- C_{H}2; (x) V_{L}-C_{H}3; (xi) V_{L}-C_{H}1-C_{H}2; (xii) V_{L}-C_{H}1-C_{H}2-C_{H}3; (xiii) V_{L}-C_{H}2-C_{H}3; and (xiv) V_{L}-C_{L}. In any configuration of variable and constant domains, including any of the exemplary configurations listed above, the variable and constant domains may be either directly linked to one another or may be linked by a full or partial hinge or linker region. A hinge region may consist of at least 2 (e.g., 5, 10, 15, 20, 40, 60 or more) amino acids, which result in a flexible or semi-flexible linkage between adjacent variable and/or constant domains in a single polypeptide molecule. Moreover, an antigen-binding fragment of an antibody of the present disclosure may comprise a homo-dimer or hetero-dimer (or other multimer) of any of the variable and constant domain configurations listed above in non-covalent association with one another and/or with one or more monomeric V_{H} or V_{L} domain (e.g., by disulfide bond(s)).

As used herein, an “antibody” refers to an immunoglobulin molecule comprising four polypeptide chains, two heavy chains (HC) and two light chains (LC) inter-connected by disulfide bonds. Each heavy chain (HC) comprises a heavy chain variable region (abbreviated herein as HCVR or V_{H}) and a heavy chain constant region (e.g., IgG, IgG1 or IgG4). The heavy chain constant region comprises three domains, C_{H}1, C_{H}2 and C_{H}3. Each light chain (LC) comprises a light chain variable region (abbreviated herein as LCVR or V_{L}) and a light chain constant region (e.g., lambda or kappa). The light chain constant region comprises one domain (C_{L}1). The HCVR and LCVR regions can be further subdivided into regions of hypervariability, termed complementarity determining regions (CDRs), interspersed with regions that are more conserved, termed framework regions (FR). Each HCVR and LCVR includes three CDRs and four FRs, arranged from amino-terminus to carboxy-terminus in the following order: FR1, CDR1, FR2, CDR2, FR3, CDR3, FR4. A heavy chain CDR may be referred to as HCDR and a light chain CDR may be referred to as LCDR. In different embodiments, the FRs of an antibody (or antigen-binding fragment thereof) may be identical to the human germline sequences, or may be naturally or artificially modified.

As used herein, an “antigen-binding arm” or “arm” of a Y-shaped IgG antibody (e.g., a CD22 or 4-1BB binding arm) refers to a structural portion of the antibody that confers binding specificity to the antigen. An antigen-binding arm of an IgG antibody has a heavy chain (HC) associated with a light chain (LC).

As used herein, the expression “multispecific antigen-binding molecule” means a protein, polypeptide, antibody, fragment of an antibody, or other molecular complex comprising a first antigen-binding arm and a second antigen-binding arm. A multispecific antigen-binding molecule specifically binds to two or more different antigens (e.g., CD22 and 4-1BB). Each antigen-binding arm within the multispecific antigen-binding molecule comprises at least one CDR that alone, or in combination with one or more additional CDRs and/or FRs, specifically binds to a particular antigen. In some embodiments, antigen-binding fragments of an antibody may be derived, e.g., from full antibody molecules using any suitable standard techniques such as proteolytic digestion or recombinant genetic engineering techniques involving the manipulation and expression of DNA encoding antibody variable and optionally constant domains. Non-limiting examples of antigen-binding fragments include: (i) Fab fragments; (ii) F(ab′)₂ fragments; (iii) Fd fragments; (iv) Fv fragments; (v) single-chain Fv (scFv) molecules; and (vi) dAb fragments.

The present disclosure provides multispecific antigen-binding molecules that bind CD22 and 4-1BB. Such multispecific antigen-binding molecules are also referred to herein as “multispecific CD22×4-1BB binding molecules” or “anti-CD22×anti-4-1BB,” “anti-4-1BB×anti-CD22,” “anti-CD22/anti-4-1BB,” “anti-4-1BB/anti-CD22,” “anti-CD22×4-1BB,” “anti-4-1BB×CD22,” “CD22×4-1BB,” “4-1BB×CD22” antigen-binding molecules or similar terminology. Such molecules may be referred to herein as “anti-CD22” or “CD22.” The present disclosure also includes multispecific antibodies and antigen-binding fragments thereof that specifically bind CD22 and 4-1BB. Such molecules may be referred to herein The multispecific antigen-binding molecules disclosed herein contain three antigen-binding domains, D1, D2, and D3. The D1 domain specifically binds CD22. The D2 domain and the D3 domain each specifically bind 4-1BB. In some aspects, D2 and D3 bind different 4-1BB epitopes. In some aspects, D2 and D3 bind the same 4-1BB epitopes. In some aspects, the multispecific antigen-binding molecules provided herein contain two antigen-binding arms, A1 and A2; A1 contains D1, and A2 contains D2 and D3. In some aspects, D2 is linked to D3 via a linker, forming stacked antigen-binding domains on the A2 arm. The combination of the A1 arm and the stacked A2 arm is termed a 1+2 format. The antigen-binding domains of A2 may be contained in Fabs, an “outer” Fab2 and an “inner” Fab3. In some embodiments, D2 is contained in Fab2 and D3 is contained in Fab3. In some embodiments, the Fab2 and Fab3 of the anti-CD22×anti-4-1BB 1+2 multispecific antigen-binding molecule are connected via a linker from the C-terminus of the C_{H}1 of Fab2 (“C_{H}1-2”) to the N-terminus of the V_{H} of Fab3 (“V_{H}-3”). In some aspects, the amino acid sequences of D2 and D3 contain heavy chain variable regions (HCVR) that are identical or substantially similar, i.e., less than 5, or less than 4, or less than 3 amino acid differences in a HCVR or a heavy chain complementarity determining region (HCDR). In some aspects, the amino acid sequences of D2 and D3 HCVRs are identical or substantially similar, i.e., 2 or 1 amino acid differences in a HCVR or a HCDR. In some aspects, the D2 and D3 HCVRs have different antigen-binding sequences.

As used herein, “recombinant” antibodies or antigen-binding fragments thereof refer to such molecules created, expressed, isolated or obtained by technologies or methods known in the art as recombinant DNA technology which include, e.g., DNA splicing and transgenic expression. The term includes antibodies or antigen-binding fragments thereof expressed in a non-human mammal (including transgenic non-human mammals, e.g., transgenic mice), or a host cell (e.g., Chinese hamster ovary (CHO) cell) or cellular expression system or isolated from a recombinant combinatorial human antibody library. The present disclosure includes recombinant antigen-binding proteins.

As used herein, the expression “specifically binds” or “binds specifically” refers to those antibodies or antigen-binding fragments thereof having a binding affinity to an antigen, such as CD22 or 4-1BB protein, expressed as K_{D}, of less than about 10⁻⁶ M (e.g., 10⁻⁷ M, 10⁻⁸ M, 10⁻⁹ M, 10⁻¹⁰ M, 10⁻¹¹ M or 10⁻¹² M), as measured by real-time, label free bio-layer interferometry assay, for example, at 25° C. or 37° C., e.g., an Octet® HTX biosensor, or by surface plasmon resonance, e.g., BIACORE™, or by solution-affinity ELISA.

As used herein, “anti-CD22” refers to an antigen-binding protein (or other molecule such as an antigen-binding arm or Fab), for example an antibody or antigen-binding fragment thereof, that binds specifically to CD22 and “anti-4-1BB” refers to an antigen-binding protein (or other molecule such as an antigen-binding arm or Fab), for example an antibody or antigen-binding fragment thereof, that binds specifically to 4-1BB. “Multispecific CD22×4-1BB antibody or antigen-binding fragment thereof” refers to an antibody or antigen-binding fragment thereof that binds specifically to CD22 and to 4-1BB (and, optionally, to one or more other antigens).

As used herein, the term “epitope” refers to an antigenic determinant (e.g., on CD22 or 4-1BB) that interacts with a specific antigen-binding site of an antigen-binding protein, e.g., a variable region of an antibody molecule, known as a paratope. A single antigen may have more than one epitope. Thus, different antibodies or antigen-binding fragments thereof may bind to different areas on an antigen and may have different biological effects. This term may also refer to a site on an antigen to which B and/or T cells respond and/or to a region of an antigen that is bound by an antibody. Epitopes may be defined as structural or functional. Functional epitopes are generally a subset of the structural epitopes and have those residues that directly contribute to the affinity of the interaction. Epitopes may be linear or conformational, that is, composed of non-linear amino acids. In certain embodiments, epitopes may include determinants that are chemically active surface groupings of molecules such as amino acids, sugar side chains, phosphoryl groups, or sulfonyl groups, and, in certain embodiments, may have specific three-dimensional structural characteristics, and/or specific charge characteristics.

Methods for determining the epitope of an antigen-binding molecule or polypeptide include alanine scanning mutational analysis, peptide blot analysis (Reineke (2004) Methods Mol. Biol. 248: 443-63), peptide cleavage analysis, crystallographic studies and NMR analysis. In addition, methods such as epitope excision, epitope extraction and chemical modification of antigens can be employed (Tomer (2000) Prot. Sci. 9: 487-496). Another method that can be used to identify the amino acids within a polypeptide with which an antigen-binding molecule or polypeptide interacts is hydrogen/deuterium exchange detected by mass spectrometry. See, e.g., Ehring (1999) Analytical Biochemistry 267: 252-259; Engen and Smith (2001) Anal. Chem. 73: 256A-265A.

The present disclosure includes antigen-binding molecules that compete for binding to CD22 with another antibody or antigen-binding fragment thereof. As used herein, an antigen-binding molecule that “competes” refers to an antigen-binding protein (e.g., antibody or antigen-binding fragment thereof) that binds to an antigen and inhibits or blocks the binding of another antigen-binding protein (e.g., antibody or antigen-binding fragment thereof) to the antigen. Unless otherwise stated, the term also includes competition between two antigen-binding molecules, in both orientations, i.e., a first antibody that binds antigen and blocks binding by a second antibody and vice versa. In some embodiments, competition occurs in one such orientation. In some embodiments, the first antigen-binding protein and second antigen-binding protein may bind to the same epitope. Alternatively, in some embodiments, the first and second antigen-binding proteins may bind to different but overlapping or non-overlapping epitopes, wherein binding of one inhibits or blocks the binding of the second antibody, e.g., via steric hindrance. Competition between antigen-binding proteins may be measured by known methods, such as a real-time, label-free bio-layer interferometry assay. Also, binding competition between antigen-binding proteins (e.g., monoclonal antibodies (mAbs)) can be determined using a real time, label-free bio-layer interferometry assay on an Octet RED384 biosensor (Pall ForteBio Corp.).

Typically, an antibody or antigen-binding fragment of the disclosure which is modified in some way retains the ability to specifically bind to CD22 and 4-1BB, e.g., retains at least 10% of its CD22 and 4-1BB binding activity (when compared to the parental antibody) when that activity is expressed on a molar basis. Preferably, an antibody or antigen-binding fragment of the disclosure retains at least 20%, at least 50%, at least 70%, at least 80%, at least 90%, at least 95% or 100% of the CD22 and 4-1BB binding affinity as the parental antibody. In some embodiments, an antibody or antigen-binding fragment of the disclosure may include conservative or non-conservative amino acid substitutions (referred to as “conservative variants” or “function conserved variants” of the antibody) that do not substantially alter its biologic activity.

As used herein, the singular forms “a,” “an,” and “the” include plural reference unless the context clearly dictates otherwise. As used herein, the terms “including,” “comprising,” “containing,” or “having” and variations thereof are meant to encompass the items listed thereafter and equivalents thereof as well as additional subject matter unless otherwise noted. As used herein, the phrases “in one embodiment,” “in various embodiments,” “in some embodiments,” and the like are used repeatedly. Such phrases do not necessarily refer to the same embodiment, but they may unless the context dictates otherwise. As used herein, the terms “and/or” or “/” means any one of the items, any combination of the items, or all of the items with which this term is associated.

The following references relate to BLAST algorithms often used for sequence analysis: Altschul et al. (2005) FEBS J. 272(20): 5101-5109; Altschul et al., (1990) J. Mol. Biol. 215:403-410; Gish et al., (1993) Nature Genet. 3:266-272; Madden et al., (1996) Meth. Enzymol. 266:131-141; Altschul et al., (1997) Nucleic Acids Res. 25:3389-3402; Zhang (1997) Genome Res. 7:649-656; Wootton et al., (1993) Comput. Chem. 17:149-163; Hancock et al., (1994) Comput. Appl. Biosci. 10:67-70.

The following references relate to Alignment Scoring Systems: Dayhoff et al., “A Model of Evolutionary Change in Proteins,” ATLAS OF PROTEIN SEQUENCE AND STRUCTURE, (1978) vol. 5, suppl. 3., Dayhoff (ed.), pp. 345-352, Natl. Biomed. Res. Found., Washington, DC; Schwartz et al., “Matrices for Detecting Distant Relationships,” ATLAS OF PROTEIN SEQUENCE AND STRUCTURE, (1978) vol. 5, suppl. 3.” Dayhoff (ed.), pp. 353-358, Natl. Biomed. Res. Found., Washington, DC; Altschul (1991) J. Mol. Biol. 219:555-565; States, (1991) Methods 3:66-70; Henikoff et al., (1992) Proc. Natl. Acad. Sci. USA 89:10915-10919; Altschul et al., (1993) J. Mol. Evol. 36:290-300.

The following references relate to Alignment Statistics: Karlin et al., (1990) Proc. Natl. Acad. Sci. USA 87:2264-2268; Karlin et al., (1993) Proc. Natl. Acad. Sci. USA 90:5873-5877; Dembo et al., (1994) Ann. Prob. 22:2022-2039; Altschul, “Evaluating the Statistical Significance of Multiple Distinct Local Alignments,” THEORETICAL AND COMPUTATIONAL METHODS IN GENOME RESEARCH (Suhai, ed.), (1997) pp. 1-14, Plenum, NY.

In some embodiments, the disclosure includes a multispecific antibody that has an effector arm and a targeting arm. The effector arm may be the first antigen-binding arm that binds to 4-1BB on effector cells (e.g., T cells). The targeting arm may be the second antigen-binding arm that binds to an antigen (e.g., CD22 on target cells (e.g., tumor cells)). In some embodiments of the present disclosure, the effector arm binds to 4-1BB and the targeting arm binds to CD22.

In some embodiments of the disclosed multispecific antigen-binding molecules, the first antigen-binding domain, the second or more antigen-binding domains may be directly or indirectly connected to one another. Alternatively, the first antigen-binding domain and the second or more antigen-binding domains may each be connected to a separate multimerizing domain. The association of one multimerizing domain with another multimerizing domain facilitates the association between the two or more antigen-binding domains, thereby forming a multispecific antigen-binding molecule.

As used herein, a “multimerizing domain” refers to any macromolecule, protein, polypeptide, peptide, or amino acid that has the ability to associate with a second multimerizing domain of the same or similar structure. For example, a multimerizing domain may be a polypeptide comprising an immunoglobulin C_{H}3 domain. A non-limiting example of a multimerizing domain is an Fc portion of an immunoglobulin (comprising a C_{H}2-C_{H}3 domain), e.g., an Fc domain of an IgG selected from the isotypes IgG1, IgG2, IgG3, and IgG4, as well as any allotype within each isotype group. The Fc domain may comprise wild-type or modified IgG isotype.

In some embodiments, the disclosed multispecific antibodies or antigen-binding fragments thereof comprise two multimerizing domains, e.g., two Fc domains that are each individually part of a separate antibody heavy chain. The first and second multimerizing domains may be of the same IgG isotype such as, e.g., IgG1/IgG1, IgG2/IgG2, IgG4/IgG4. Alternatively, the first and second multimerizing domains may be of different IgG isotypes such as, e.g., IgG1/IgG2, IgG1/IgG4, IgG2/IgG4, etc.

In certain embodiments, the multimerizing domain is an Fc fragment or an amino acid sequence of 1 to about 200 amino acids in length containing at least one cysteine residues. In other embodiments, the multimerizing domain is a cysteine residue, or a short cysteine containing peptide. Other multimerizing domains include peptides or polypeptides comprising or consisting of a leucine zipper, a helix-loop motif, or a coiled-coil motif.

Any multispecific antibody format or technology may be used to make the multispecific antibodies of the present disclosure. For example, an antibody or antigen-binding fragment thereof having a first or more antigen-binding specificity can be functionally linked (e.g., by chemical coupling, genetic fusion, noncovalent association or otherwise) to one or more other molecular entities, such as another antibody or antibody fragment having a second or more antigen-binding specificity to produce a multispecific antigen-binding molecule. Specific exemplary multispecific formats that can be used in the context of the present disclosure include, without limitation, e.g., scFv-based or diabody multispecific formats, IgG-scFv fusions, dual variable domain (OVO)-Ig, Quadroma, knobs-into-holes, common light chain (e.g., common light chain with knobs-intoholes, etc.), CrossMab, CrossFab, (SEEO) body, leucine zipper, Ouobody, IgG1/IgG2, dual acting Fab (OAF)-IgG, and Mab² multispecific formats (see, e.g., Klein et al. 2012, mAbs 4:6, 1-11, and references cited therein, for a review of the foregoing formats).

The multimerizing domains, e.g., Fc domains, may comprise one or more amino acid changes (e.g., insertions, deletions or substitutions) as compared to the wild-type, naturally occurring version of the Fc domain. For example, the disclosed multispecific antibodies or antigen-binding fragments thereof may comprise one or more modifications in the Fc domain that results in a modified Fc domain having a modified binding interaction (e.g., enhanced or diminished) between Fc and FcRn. In one embodiment, the multispecific antigen-binding molecule comprises a modification in a C_{H}2 or a C_{H}3 region, wherein the modification increases the affinity of the Fc domain to FcRn in an acidic environment (e.g., in an endosome where pH ranges from about 5.5 to about 6.0). Non-limiting examples of such Fc modifications include, e.g., a modification at position 250 (e.g., E or Q); 250 and 428 (e.g., L or F); 252 (e.g., LN/FIW or T), 254 (e.g., S or T), and 256 (e.g., S/R/Q/EID or T); or a modification at position 428 and/or 433 (e.g., UR/S/P/Q or K) and/or 434 (e.g., H/F or V); or a modification at position 250 and/or 428; or a modification at position 307 or 308 (e.g., 308F, V308F), and 434. In one embodiment, the modification comprises a 428L (e.g., M428L) and 434S (e.g., N434S) modification; a 428L, 2591 (e.g., V259I), and 308F (e.g., V308F) modification; a 433K (e.g., H433K) and a 434 (e.g., 434Y) modification; a 252,254, and 256 (e.g., 252Y, 254T, and 256E) modification; a 250Q and 428L modification (e.g., T250Q and M428L); and a 307 and/or 308 modification (e.g., 308F or 308P).

The present disclosure also includes multispecific antibodies comprising a first C_{H}3 domain and a second Ig C_{H}3 domain, wherein the first and second Ig C_{H}3 domains differ from one another by at least one amino acid, and wherein at least one amino acid difference reduces binding of the multispecific antibody to Protein A as compared to a multispecific antibody lacking the amino acid difference. In one embodiment, the first Ig C_{H}3 domain binds Protein A and the second Ig C_{H}3 domain contains a mutation that reduces or abolishes Protein A binding such as an H95R modification (by IMGT exon numbering; H435R by EU numbering). The second C_{H}3 may further comprise a Y96F modification (by IMGT; Y436F by EU). Further modifications that may be found within the second C_{H}3 include: D16E, L 18M, N44S, K52N, V57M, and V821 (by IMGT; D356E, L358M, N384S, K392N, V397M, and V4221 by EU) in the case of IgG1 antibodies; N44S, K52N, and V821 (IMGT; N384S, K392N, and V4221 by EU) in the case of IgG2 antibodies; and Q15R, N44S, K52N, V57M, R69K, E79Q, and V821 (by IMGT; Q355R, N384S, K392N, V397M, R409K, E419Q, and V4221 by EU) in the case of IgG4 antibodies.

In certain embodiments, the Fc domain may be chimeric, combining Fc sequences derived from more than one immunoglobulin isotype. For example, a chimeric Fc domain can comprise part or all of a C_{H}2 sequence derived from a human IgG1, human IgG2 or human IgG4 C_{H}2 region, and part or all of a C_{H}3 sequence derived from a human IgG1, human IgG2 or human IgG4. A chimeric Fc domain can also contain a chimeric hinge region. For example, a chimeric hinge may comprise an “upper hinge” sequence, derived from a human IgG1, a human IgG2 or a human IgG4 hinge region, combined with a “lower hinge” sequence, derived from a human IgG1, a human IgG2 or a human IgG4 hinge region. A particular example of a chimeric Fc domain that can be included in any of the antibodies set forth herein comprises, from N- to C-terminus: [IgG4 C_{H}1]-[IgG4 upper hinge]-[IgG2 lower hinge]-[IgG4 C_{H}2]-[IgG4 C_{H}³]. Another example of a chimeric Fc domain that can be included in any of the antibodies set forth herein comprises, from N- to C-terminus: [IgG1 C_{H}1]-[IgG1 upper hinge]-[IgG2 lower hinge]-[IgG4 C_{H}2]-[IgG1 C_{H}3]. These and other examples of chimeric Fc domains that can be included in any of the antibodies of the present disclosure are described in WO2014/022540. Chimeric Fc domains having these general structural arrangements, and variants thereof, can have altered Fc receptor binding, which in turn affects Fc effector function.

Antibodies and antigen-binding fragments of the present disclosure comprise immunoglobulin chains including the amino acid sequences specifically set forth herein (and variants thereof) as well as cellular and in vitro post-translational modifications to the antibody or fragment. For example, the present disclosure includes monospecific antibodies and antigen-binding fragments thereof that specifically bind to CD22, and multispecific antibodies and antigen-binding fragments thereof that specifically bind to CD22 and 4-1BB comprising heavy and/or light chain amino acid sequences set forth herein as well as antibodies and fragments wherein one or more asparagine, serine and/or threonine residues is glycosylated, one or more asparagine residues is deamidated, one or more residues (e.g., Met, Trp and/or His) is oxidized, the N-terminal glutamine is pyroglutamate (pyroE) and/or the C-terminal lysine or other amino acid is missing.

### Polynucleotides and Methods of Making

The present disclosure includes isolated polynucleotide molecules or sets of isolated polynucleotide molecules comprising polynucleotide sequences encoding the immunoglobulin chains of a CD22 monospecific, a 4-1BB monospecific or CD22×4-1BB multispecific antigen-binding protein. The present disclosure also includes vectors or sets of vectors comprising the polynucleotide molecules and/or a host cell (e.g., Chinese hamster ovary (CHO) cell) comprising the polynucleotide molecules, vector(s) or antigen-binding protein(s) set forth herein.

A polynucleotide molecule or sequence refers to DNA or RNA. In some embodiments, the disclosed polynucleotide molecules encode an immunoglobulin V_{H}, V_{L}, CDRs-H, CDRs-L, HC, and/or LC of an CD22 binding arm and/or a 4-1BB binding arm. Optionally, the disclosed polynucleotide molecule is operably linked to a promoter or other expression control sequence or other polynucleotide sequence. Examples of a polynucleotide molecule or set of polynucleotide molecules (e.g., DNA) of the present disclosure include a nucleotide sequence set forth in Tables 2, 4, or 7.

In general, a “promoter” or “promoter sequence” is a DNA regulatory region capable of binding an RNA polymerase in a cell (e.g., directly or through other promoter-bound proteins or substances) and initiating transcription of a coding sequence. A promoter may be operably linked to other expression control sequences, including enhancer and repressor sequences and/or with a polynucleotide of the disclosure. Examples of promoters which may be used to control gene expression include, but are not limited to, cytomegalovirus (CMV) promoter (U.S. Pat. Nos. 5,385,839 and 5,168,062), the SV40 early promoter region (Benoist et al., (1981) Nature 290:304-310), the promoter contained in the 3′ long terminal repeat of Rous sarcoma virus (Yamamoto et al., (1980) Cell 22:787-797), the herpes thymidine kinase promoter (Wagner, et al., (1981) Proc. Natl. Acad. Sci. USA 78:1441-1445), the regulatory sequences of the metallothionein gene (Brinster, et al., (1982) Nature 296:39-42); prokaryotic expression vectors such as the beta-lactamase promoter (VIIIa-Komaroff, et al., (1978) Proc. Natl. Acad. Sci. USA 75:3727-3731), or the tac promoter (DeBoer, et al., (1983) Proc. Natl. Acad. Sci. USA 80:21-25); see also “Useful proteins from recombinant bacteria” in Scientific American (1980) 242:74-94; and promoter elements from yeast or other fungi such as the Ga/4 promoter, the ADC (alcohol dehydrogenase) promoter, PGK (phosphoglycerol kinase) promoter or the alkaline phosphatase promoter.

A polynucleotide encoding a polypeptide is “operably linked” to a promoter or other expression control sequence when, in a cell or other expression system, the sequence directs RNA polymerase mediated transcription of the coding sequence into RNA, preferably mRNA, which then may be RNA spliced (if it contains introns) and, optionally, translated into a protein encoded by the coding sequence.

Eukaryotic and prokaryotic host cells, including mammalian cells, may be used as hosts for expression of a CD22 monospecific antibody or antigen-binding arm thereof or CD22×4-1BB multispecific antibody or antigen-binding arm thereof. Such host cells are well known in the art and many are available from the American Type Culture Collection (ATCC). These host cells include, inter alia, Chinese hamster ovary (CHO) cells, NSO, SP2 cells, HeLa cells, baby hamster kidney (BHK) cells, monkey kidney cells (COS), human hepatocellular carcinoma cells (e.g., Hep G2), A549 cells, 3T3 cells, HEK-293 cells and a number of other cell lines. Mammalian host cells include human, mouse, rat, dog, monkey, pig, goat, bovine, horse and hamster cells. Other cell lines that may be used include insect cell lines (e.g., Spodoptera frugiperda or Trichoplusia ni), amphibian cells, bacterial cells, plant cells and fungal cells. Fungal cells include yeast and filamentous fungus cells. The present disclosure includes an isolated host cell (e.g., a CHO cell or any type of host cell set forth above) comprising an CD22×4-1BB multispecific antigen-binding protein of the present disclosure, such as REGN9220, REGN9289, and REGN17596, shown in Table 4, or one or more polynucleotide molecules encoding an immunoglobulin (Ig) heavy and/or light chain thereof; and/or one or more polynucleotides encoding the CD22 binding arm and/or 4-1BB binding arm of an antigen-binding protein of the present disclosure, such as the CD22×4-1BB antibodies shown in Table 8.

The present disclosure also includes a cell which is expressing an CD22 and/or 4-1BB or an antigenic fragment or fusion thereof (e.g., His₆, Fc and/or myc) which is bound by an CD22 or CD22×4-1BB antigen-binding protein of the present disclosure (e.g., an antibody or antigen-binding fragment thereof), for example, REGN9220, REGN9289, and REGN17596 disclosed herein.

There are several methods by which to produce recombinant antibodies which are known in the art. See, e.g., U.S. Pat. No. 4,816,567. Transformation can be by any known method for introducing polynucleotides into a host cell. Methods for introduction of heterologous polynucleotides into mammalian cells are known. In addition, nucleic acid molecules may be introduced into mammalian cells by viral vectors. Methods of transforming cells are known. See, e.g. U.S. Pat. Nos. 4,399,216; 4,912,040; 4,740,461; 4,959,455.

The present disclosure includes recombinant methods for making an anti-CD22×anti-4-1BB antibody, such as an immunoglobulin chain thereof, comprising: (i) introducing, into a host cell, one or more polynucleotides encoding the light and heavy immunoglobulin chains encoding the CD22×4-1BB antibody's antigen-binding arms for example, wherein the polynucleotide is in a vector; and/or integrates into the host cell chromosome and/or is operably linked to a promoter; (ii) culturing the host cell (e.g., CHO) under conditions favorable to expression of the polynucleotide, and (iii) optionally, isolating the antibody or chain from the host cell and/or medium in which the host cell is grown. The present disclosure also includes CD22×4-1BB antibodies or antigen-binding fragments thereof, which are the products of production methods and, optionally, the purification methods set forth herein.

In some embodiments, the disclosed CD22×4-1BB antibodies or antigen-binding fragments thereof may be made by a method that includes purifying the antibody or antigen-binding fragment thereof, e.g., by column chromatography, precipitation and/or filtration. The present disclosure also includes the product of such a method.

### Sequence Variants

In some embodiments, the disclosed antibodies comprise one or more amino acid substitutions, insertions and/or deletions in the framework and/or CDR regions of the heavy and light chain variable domains as compared to the corresponding germline sequences from which the individual antigen-binding domains were derived. Such mutations can be readily ascertained by comparing the amino acid sequences disclosed herein to germ line sequences available from, for example, public antibody sequence databases.

In some embodiments, the disclosed antigen-binding fragments of antibodies are derived from any of the exemplary amino acid sequences disclosed herein, wherein one or more amino acids within one or more framework and/or CDR regions are mutated to the corresponding residue(s) of the germline sequence from which the antibody was derived, or to the corresponding residue(s) of another human germline sequence, or to a conservative amino acid substitution of the corresponding germline residue(s) (such sequence changes are referred to herein collectively as “germline mutations”). In certain embodiments, all of the framework and/or CDR residues within the V_{H} and/or V_{L} domains are mutated back to the residues found in the original germline sequence from which the antigen-binding domain was originally derived. In other embodiments, only certain residues are mutated back to the original germline sequence, e.g., only the mutated residues found within the first 8 amino acids of FR1 or within the last 8 amino acids of FR4, or only the mutated residues found within CDR1, CDR2 or CDR3. In some embodiments, one or more of the framework and/or CDR residue(s) are mutated to the corresponding residue(s) of a different germline sequence (i.e., a germline sequence that is different from the germ line sequence from which the antigen-binding domain was originally derived). Furthermore, the antigen-binding domains may contain any combination of two or more germline mutations within the framework and/or CDR regions, e.g., wherein certain individual residues are mutated to the corresponding residue of a particular germ line sequence while certain other residues that differ from the original germ line sequence are maintained or are mutated to the corresponding residue of a different germline sequence. Once obtained, antigen-binding domains that contain one or more germline mutations can be easily tested for one or more desired properties such as improved binding specificity, increased binding affinity, improved or enhanced antagonistic or agonistic biological properties (as the case may be), reduced immunogenicity, etc.

### Antibodies Comprising Fc Variants

According to certain embodiments of the present disclosure, anti-CD22×anti-4-1BB bispecific antibodies are provided comprising an Fc domain comprising one or more mutations which enhance or diminish antibody binding to the FcRn receptor, e.g., at acidic pH as compared to neutral pH. For example, the present disclosure includes antibodies comprising a mutation in the C_{H}2 or a C_{H}3 region of the Fc domain, wherein the mutation(s) increases the affinity of the Fc domain to FcRn in an acidic environment (e.g., in an endosome where pH ranges from about 5.5 to about 6.0). Such mutations may result in an increase in serum half-life of the antibody when administered to an animal. Non-limiting examples of such Fc modifications include, e.g., a modification at position:
250 (e.g., E or Q);
250 and 428 (e.g., L or F);
252 (e.g., L/Y/F/W or T),
254 (e.g., S or T), and/or
256 (e.g., S/R/Q/E/D or T);
   or a modification at position:
428 and/or 433 (e.g., H/L/R/S/P/Q or K) and/or
434 (e.g., H/F or Y);
   or a modification at position:
250 and/or 428;
   or a modification at position:
307 or 308 (e.g., 308F, V308F), and/or
434.

In one embodiment, the modification comprises one or more of the following:
428L (e.g., M428L) and 434S (e.g., N434S) modification;
a 428L, 259I (e.g., V259I), and 308F (e.g., V308F) modification;
a 433K (e.g., H433K) and a 434 (e.g., 434Y) modification;
a 252, 254, and 256 (e.g., 252Y, 254T, and 256E) modification;
a 250Q and 428L modification (e.g., T250Q and M428L); and/or
a 307 and/or 308 modification (e.g., 308F or 308P).

For example, the disclosed CD22×4-1BB bispecific antibodies may comprise an Fc domain comprising one or more pairs or groups of mutations selected from:
250Q and 248L (e.g., T250Q and M248L);
252Y, 254T and 256E (e.g., M252Y, S254T and T256E);
428L and 434S (e.g., M428L and N434S); and
433K and 434F (e.g., H433K and N434F).

The present disclosure also includes bispecific antibodies comprising a first C_{H}3 domain and a second Ig C_{H}3 domain, wherein the first and second Ig C_{H}3 domains differ from one another by at least one amino acid, and wherein at least one amino acid difference reduces binding of the bispecific antibody to Protein A as compared to a bi-specific antibody lacking the amino acid difference. In one embodiment, the first Ig C_{H}3 domain binds Protein A and the second Ig C_{H}3 domain contains a mutation that reduces or abolishes Protein A binding such as an H95R modification (by IMGT exon numbering; H435R by EU numbering). The second C_{H}3 may further comprise a Y96F modification (by IMGT; Y436F by EU). See, e.g., U.S. Pat. No. 8,586,713. Further modifications that may be found within the second C_{H}3 include: D16E, L18M, N44S, K52N, V57M, and V82I (by IMGT; D356E, L358M, N384S, K392N, V397M, and V422I by EU) for IgG1 antibodies; N44S, K52N, and V82I (IMGT; N384S, K392N, and V422I by EU) for IgG2 antibodies; and Q15R, N44S, K52N, V57M, R69K, E79Q, and V82I (by IMGT; Q355R, N384S, K392N, V397M, R409K, E419Q, and V422I by EU) for IgG4 antibodies.

All possible combinations of the foregoing Fc domain mutations, and other mutations within the antibody variable domains disclosed herein, are within the scope of the present disclosure.

### Biological Characteristics of the Multispecific Antibodies and Antigen-Binding Molecules

The present disclosure includes antibodies and antigen-binding fragments thereof bind human CD22 and 4-1BB with high affinity. The present disclosure also includes antibodies and antigen-binding fragments thereof that bind human CD22 and/or PSMA with medium affinity or low affinity, depending on the therapeutic context and particular targeting properties that are desired. For example, in the context of a multispecific antigen-binding molecule, wherein one arm binds 4-1BB and another arm binds a target antigen (e.g., CD22), it may be desirable for the target antigen-binding arm to bind the target antigen with high affinity while the anti-4-1BB arm binds 4-1BB with only moderate or low affinity. In this manner, preferential targeting of the antigen-binding molecule to cells expressing the target antigen may be achieved while avoiding general/untargeted 4-1BB binding and the consequent adverse side effects associated therewith.

According to certain embodiments, the present disclosure includes antibodies and antigen-binding fragments of antibodies that bind human 4-1BB (e.g., at 25° C.) with a K_{D} of less than about 150 nM as measured by surface plasmon resonance, e.g., using an assay format as defined in Example 2 herein. In certain embodiments, the antibodies or antigen-binding fragments of the present disclosure bind 4-1BB with a K_{D} of less than about 140 nM, less than about 120 nM, less than about 100 nM, less than about 80 nM, less than about 60 nM, less than about 40 nM, less than about 20 nM, less than about 10 nM, less than about 5 nM, or less than about 1 nM as measured by surface plasmon resonance, e.g., using an assay format as defined in Example 2 herein, or a substantially similar assay. In certain embodiments, the antibodies or antigen-binding fragments of the present disclosure bind 4-1BB with a K_{D} of about 1 nM to about 120 nM.

The present disclosure also includes antibodies and antigen-binding fragments thereof that bind 4-1BB with a dissociative half-life (t½) of greater than about 1 minute as measured by surface plasmon resonance at 25° C. or 37° C., e.g., using an assay format as defined in Example 2 herein, or a substantially similar assay. In certain embodiments, the antibodies or antigen-binding fragments of the present disclosure bind 4-1BB with a t½ of greater than about 2 minutes, greater than about 5 minutes, greater than about 10 minutes, greater than about 20 minutes, greater than about 40 minutes, greater than about 80 minutes, greater than about 100 minutes, greater than about 120 minutes, greater than about 150 minutes, or greater than about 170 minutes, as measured by surface plasmon resonance at 25° C. or 37° C., e.g., using an assay format as defined in Example 2 herein, or a substantially similar assay.

According to certain embodiments, the present disclosure includes antibodies and antigen-binding fragments of antibodies that bind human CD22 (e.g., at 25° C.) with a K_{D} of less than about 40 nM as measured by surface plasmon resonance, e.g., using an assay format as defined in Example 2 herein. In certain embodiments, the antibodies or antigen-binding fragments of the present disclosure bind CD22 with a K_{D} of less than about 30 nM, less than about 20 nM, less than about 10 nM, or less than about 5 nM as measured by surface plasmon resonance, e.g., using an assay format as defined in Example 2 herein, or a substantially similar assay. In certain embodiments, the antibodies or antigen-binding fragments of the present disclosure bind CD22 with a K_{D} of about 1 nM to about 20 nM.

The present disclosure also includes antibodies and antigen-binding fragments thereof that bind CD22 with a dissociative half-life (t½) of greater than about 1 minutes as measured by surface plasmon resonance at 25° C. or 37° C., e.g., using an assay format as defined in the Examples herein, or a substantially similar assay. In certain embodiments, the antibodies or antigen-binding fragments of the present disclosure bind CD22 with a t½ of greater than about 2 minutes, greater than about 5 minutes, greater than about 10 minutes, greater than about 10 minutes, greater than about 20 minutes, greater than about 40 minutes, or greater than about 80 minutes, as measured by surface plasmon resonance at 25° C. or 37° C., e.g., using an assay format as defined in Example 2 herein, or a substantially similar assay.

The present disclosure includes multispecific antigen-binding molecules (e.g., multispecific antibodies) which are capable of simultaneously binding to human 4-1BB and human CD22. According to certain embodiments, the multispecific antigen-binding molecules of the disclosure specifically interact with cells that express 4-1BB and/or CD22. The extent to which a multispecific antigen-binding molecule binds cells that express 4-1BB and/or CD22 can be assessed by fluorescence activated cell sorting (FACS), as illustrated in Example 3 herein. For example, the present disclosure includes multispecific antigen-binding molecules which specifically bind human cell lines which express 4-1BB but not CD22 (e.g., Jurkat cell genetically engineered to express 4-1BB). In some embodiments, the multispecific antigen-binding molecules bind to 4-1BB-expressing human or cynomolgus T cells with an EC50 value less than 1×10⁻⁷ M. In some embodiments, the multispecific antigen-binding molecules bind to 4-1BB-expressing human or cynomolgus T cells with an EC50 value of 1×10⁻¹¹ M to 1×10⁻⁷ M. In certain embodiments, the multispecific antigen-binding molecules bind to 4-1BB-expressing human or cynomolgus T cells with an EC50 value of 1×10⁻¹⁰ M to 1×10⁻⁸ M. In certain embodiments, the multispecific antigen-binding molecules bind to the surface of cell lines expressing CD22 with an EC₅₀ of less than about 5×10⁻⁹ M. The binding of the multispecific antigen-binding molecules to the surface of cells or cell lines can be measured by an in vitro FACS binding assay as described in Example 3.

The present disclosure includes CD22×4-1BB multispecific antigen-binding molecules which are capable of depleting tumor cells in a subject. In some embodiments, a single administration of the antigen-binding molecule to a subject at a therapeutically effective dose causes a reduction in the number of tumor cells in the subject.

The present disclosure includes CD22×4-1BB multispecific antigen-binding molecules which are capable of binding to CD22 expressed on cell surfaces. A variety of B-cell tumor cells express CD22. Examples of B-cell cancers are Diffuse large B-cell lymphoma (DLBCL), Follicular lymphoma, Chronic lymphocytic leukemia (CLL)/small lymphocytic lymphoma (SLL), Mantle cell lymphoma (MCL), Marginal zone lymphomas, Burkitt lymphoma, Lymphoplasmacytic lymphoma (Waldenstrom macroglobulinemia), and Hairy cell leukemia. As such, the antigen-binding molecules of the disclosure are useful in treating a multitude of B-cell malignancies.

The present disclosure includes CD22×4-1BB multispecific antigen-binding molecules which are capable of activating T cells by engaging CD22 on target cells and, in the case of bispecific antigen-binding molecules, 4-1BB on T cells. As such, the antigen-binding molecules of the disclosure are useful in promoting a T-cell mediated immune response.

### Epitope Mapping and Related Technologies

The epitope on CD22 or 4-1BB to which the antigen-binding molecules of the present disclosure may bind may consist of a single contiguous sequence of 3 or more (e.g., 3, 4, 5, 6, 7, 8, 9, 10, 11, 12, 13, 14, 15, 16, 17, 18, 19, 20 or more) amino acids of a CD22 or 4-1BB protein. Alternatively, the epitope may consist of a plurality of non-contiguous amino acids (or amino acid sequences) of CD22 or 4-1BB. The antigen-binding molecules of the disclosure may interact with amino acids contained within a CD22 or 4-1BB monomer, or may interact with amino acids on two different CD22 or 4-1BB chains of a CD22 or 4-1BB dimer. The term “epitope,” as used herein, refers to an antigenic determinant that interacts with a specific antigen-binding site in the variable region of an antibody molecule known as a paratope. A single antigen may have more than one epitope. Thus, different antibodies may bind to different areas on an antigen and may have different biological effects. Epitopes may be either conformational or linear. A conformational epitope is produced by spatially juxtaposed amino acids from different segments of the linear polypeptide chain. A linear epitope is one produced by adjacent amino acid residues in a polypeptide chain. In certain circumstance, an epitope may include moieties of saccharides, phosphoryl groups, or sulfonyl groups on the antigen.

Various techniques known to persons of ordinary skill in the art can be used to determine whether an antigen-binding domain of an antibody “interacts with one or more amino acids” within a polypeptide or protein. Exemplary techniques that can be used to determine an epitope or binding domain of a particular antibody or antigen-binding domain include, e.g., routine crossblocking assay such as that described in ANTIBODIES, Harlow & Lane (Cold Spring Harbor Press, Cold Spring Harb., NY), point mutagenesis (e.g., alanine scanning mutagenesis, arginine scanning mutagenesis, etc.), peptide blots analysis (Reineke, 2004, Methods Mol Biol 248:443-463), protease protection, and peptide cleavage analysis. In addition, methods such as epitope excision, epitope extraction and chemical modification of antigens can be employed (Tomer, 2000, Protein Science 9:487-496). Another method that can be used to identify the amino acids within a polypeptide with which an antibody interacts is hydrogen/deuterium exchange detected by mass spectrometry. In general terms, the hydrogen/deuterium exchange method involves deuterium-labeling the protein of interest, followed by binding the antibody to the deuterium-labeled protein. Next, the protein/antibody complex is transferred to water to allow hydrogen-deuterium exchange to occur at all residues except for the residues protected by the antibody (which remain deuterium-labeled). After dissociation of the antibody, the target protein is subjected to protease cleavage and mass spectrometry analysis, thereby revealing the deuterium-labeled residues which correspond to the specific amino acids with which the antibody interacts. See, e.g., Ehring (1999) Analytical Biochemistry 267(2):252-259; Engen and Smith (2001) Anal. Chem. 73:256A-265A. Alternatively, in certain embodiments, the protein of interest binds to the antibody, followed by hydrogen-deuterium exchange. After dissociation of the antibody, the target protein is subjected to protease cleavage and mass spectrometry analysis, thereby revealing the non-deuterium-labeled residues which correspond to the specific amino acids with which the antibody interacts. X-ray crystal structure analysis can also be used to identify the amino acids within a polypeptide with which an antibody interacts.

The present disclosure further includes anti-CD22 and anti-4-1BB antibodies that bind to the same epitope as any of the specific exemplary antibodies described herein (e.g., antibodies comprising any of the amino acid sequences as set forth in Tables 1, 3, and 6).

The present disclosure also includes bispecific antigen-binding molecules comprising a first antigen-binding domain that specifically binds human CD22, and a second antigen-binding fragment that specifically binds human 4-1BB, wherein the first antigen-binding domain binds to the same epitope on CD22 as any of the specific exemplary CD22-specific antigen-binding domains described herein, and/or wherein the second antigen-binding domain binds to the same epitope on 4-1BB as any of the specific exemplary 4-1BB-specific antigen-binding domains described herein. Likewise, the present disclosure also includes multispecific antigen-binding molecules comprising a first antigen-binding domain that specifically binds human CD22, and a second and third antigen-binding domain that specifically binds human 4-1BB, wherein the first antigen-binding domain competes for binding to CD22 with any of the specific exemplary CD22-specific antigen-binding domains described herein, and/or wherein the second and/or third antigen-binding domain competes for binding to 4-1BB with any of the specific exemplary 4-1BB-specific antigen-binding domains described herein.

One can easily determine whether a particular antigen-binding molecule (e.g., antibody) or antigen-binding domain thereof binds to the same epitope as, or competes for binding with, a reference antigen-binding molecule of the present disclosure by using routine methods known in the art. For example, to determine if a test antibody binds to the same epitope on 4-1BB (or CD22) as a reference bispecific antigen-binding molecule of the present disclosure, the reference bispecific molecule is first allowed to bind to a 4-1BB protein (or CD22 protein). Next, the ability of a test antibody to bind to the 4-1BB (or CD22) molecule is assessed. If the test antibody is able to bind to 4-1BB (or CD22) following saturation binding with the reference bispecific antigen-binding molecule, it can be concluded that the test antibody does not compete for binding to 4-1BB (or CD22) with the reference bispecific antigen-binding molecule and/or that there is steric interference between antibodies that are binding different sites on the antigen. On the other hand, if the test antibody is not able to bind to the 4-1BB (or CD22) molecule following saturation binding with the reference bispecific antigen-binding molecule, then the test antibody competes for binding to 4-1BB (or CD22) with the reference bispecific antigen-binding molecule of the disclosure. Additional routine experimentation (e.g., peptide mutation and binding analyses) can then be carried out to confirm whether the observed lack of binding of the test antibody is in fact due to binding to the same epitope as the reference bispecific antigen-binding molecule or if steric blocking (or another phenomenon) is responsible for the lack of observed binding. Experiments of this sort can be performed using ELISA, RIA, Biacore, flow cytometry or any other quantitative or qualitative antibody-binding assay available in the art. In accordance with certain embodiments of the present disclosure, two antigen-binding proteins compete for binding to an antigen if, e.g., a 1-, 5-, 10-, 20- or 100-fold excess of one antigen-binding protein inhibits binding of the other by at least 50% but preferably 75%, 90% or even 99% as measured in a competitive binding assay (see, e.g., Junghans et al., Cancer Res. 1990:50:1495-1502). Alternatively, two antigen-binding proteins may bind to the same epitope if essentially all amino acid mutations in the antigen that reduce or eliminate binding of one antigen-binding protein reduce or eliminate binding of the other. Two antigen-binding proteins may have “overlapping epitopes” if only a subset of the amino acid mutations that reduce or eliminate binding of one antigen-binding protein reduce or eliminate binding of the other.

To determine if an antibody or antigen-binding domain thereof competes for binding with a reference antigen-binding molecule, the above-described binding methodology is performed in two orientations: In a first orientation, the reference antigen-binding molecule is allowed to bind to a 4-1BB protein (or CD22 protein) under saturating conditions followed by assessment of binding of the test antibody to the 4-1BB (or CD22) molecule. In a second orientation, the test antibody is allowed to bind to a 4-1BB (or CD22) molecule under saturating conditions followed by assessment of binding of the reference antigen-binding molecule to the 4-1BB (or CD22) molecule. If, in both orientations, only the first (saturating) antigen-binding molecule is capable of binding to the 4-1BB (or CD22) molecule, then it is concluded that the test antibody and the reference antigen-binding molecule compete for binding to 4-1BB (or CD22). As will be appreciated by a person of ordinary skill in the art, an antibody that competes for binding with a reference antigen-binding molecule may not necessarily bind to the same epitope as the reference antibody, but may sterically block binding of the reference antibody by binding an overlapping or adjacent epitope.

### Preparation of Antigen-Binding Domains and Construction of Multispecific Molecules

Antigen-binding domains specific for particular antigens can be prepared by any antibody generating technology known in the art. Once obtained, two or more different antigen-binding domains, specific for two or more different antigens (e.g., CD22 and 4-1BB), can be appropriately arranged relative to one another to produce a multispecific antigen-binding molecule of the present disclosure using routine methods. (A discussion of exemplary multispecific antibody formats that can be used to construct the bispecific antigen-binding molecules of the present disclosure is provided elsewhere herein.) In certain embodiments, one or more of the individual components (e.g., heavy and light chains) of the multispecific antigen-binding molecules of the disclosure are derived from chimeric, humanized, or fully human antibodies. Methods for making such antibodies are well known in the art. For example, one or more of the heavy and/or light chains of the multispecific antigen-binding molecules of the present disclosure can be prepared using VELOCIMMUNE™ technology. Using VELOCIMMUNE™ technology (or any other human antibody generating technology), high affinity chimeric antibodies to a particular antigen (e.g., CD22 or 4-1BB) are initially isolated having a human variable region and a mouse constant region. The antibodies are characterized and selected for desirable characteristics, including affinity, selectivity, epitope, etc. The mouse constant regions are replaced with a desired human constant region to generate fully human heavy and/or light chains that can be incorporated into the bispecific antigen-binding molecules of the present disclosure.

Genetically engineered animals may be used to make human bispecific antigen-binding molecules. For example, a genetically modified mouse can be used which is incapable of rearranging and expressing an endogenous mouse immunoglobulin light chain variable sequence, wherein the mouse expresses only one or two human light chain variable domains encoded by human immunoglobulin sequences operably linked to the mouse kappa constant gene at the endogenous mouse kappa locus. Such genetically modified mice can be used to produce fully human bispecific antigen-binding molecules comprising two different heavy chains that associate with an identical light chain that comprises a variable domain derived from one of two different human light chain variable region gene segments. (See, e.g., US 2011/0195454 for a detailed discussion of such engineered mice and the use thereof to produce bispecific antigen-binding molecules).

### Bioequivalents

The present disclosure encompasses antigen-binding molecules having amino acid sequences that vary from those of the described antibodies but that retain the ability to bind CD22 and 4-1BB. Such variant molecules comprise one or more additions, deletions, or substitutions of amino acids when compared to parent sequence, but exhibit biological activity that is essentially equivalent to that of the described antigen-binding molecules. Likewise, the antigen-binding molecules-encoding DNA sequences of the present disclosure encompass sequences that comprise one or more additions, deletions, or substitutions of nucleotides when compared to the disclosed sequence, but that encode an antigen-binding molecule that is essentially bioequivalent to the described antigen-binding molecules of the disclosure. Examples of such variant amino acid and DNA sequences are discussed above.

The present disclosure includes antigen-binding molecules that are bioequivalent to any of the exemplary antigen-binding molecules set forth herein. Two antigen-binding proteins or antibodies are considered bioequivalent if, for example, they are pharmaceutical equivalents or pharmaceutical alternatives having a rate and extent of absorption that do not show a significant difference when administered at the same molar dose under similar experimental conditions, either single does or multiple dose. Some antibodies will be considered equivalents or pharmaceutical alternatives if they are equivalent in the extent of their absorption but not in their rate of absorption and yet may be considered bioequivalent because such differences in the rate of absorption are intentional and are reflected in the labeling, are not essential to the attainment of effective body drug concentrations on, e.g., chronic use, and are considered medically insignificant for the particular drug product studied.

In one embodiment, two antigen-binding proteins are bioequivalent if there are no clinically meaningful differences in their safety, purity, and potency.

In one embodiment, two antigen-binding proteins are bioequivalent if a subject can be switched one or more times between the reference product and the biological product without an expected increase in the risk of adverse effects, including a clinically significant change in immunogenicity, or diminished effectiveness, as compared to continued therapy without such switching.

In one embodiment, two antigen-binding proteins are bioequivalent if they both act by a common mechanism or mechanisms of action for the condition or conditions of use, to the extent that such mechanisms are known.

Bioequivalence may be demonstrated by in vivo and in vitro methods. Bioequivalence measures include, e.g., (a) an in vivo test in humans or other mammals, in which the concentration of the antibody or its metabolites is measured in blood, plasma, serum, or other biological fluid as a function of time; (b) an in vitro test that has been correlated with and is reasonably predictive of human in vivo bioavailability data; (c) an in vivo test in humans or other mammals in which the appropriate acute pharmacological effect of the antibody (or its target) is measured as a function of time; and (d) in a well-controlled clinical trial that establishes safety, efficacy, or bioavailability or bioequivalence of an antibody.

Bioequivalent variants of the exemplary bispecific antigen-binding molecules set forth herein may be constructed by, for example, making various substitutions of residues or sequences or deleting terminal or internal residues or sequences not needed for biological activity. For example, cysteine residues not essential for biological activity can be deleted or replaced with other amino acids to prevent formation of unnecessary or incorrect intramolecular disulfide bridges upon renaturation. In other contexts, bioequivalent antibodies may include the exemplary bispecific antigen-binding molecules set forth herein comprising amino acid changes which modify the glycosylation characteristics of the antibodies, e.g., mutations which eliminate or remove glycosylation.

In some embodiments, the disclosed antibodies and antigen-binding fragments thereof comprise an antigen-binding domain with an HCVR, LCVR, and/or CDR amino acid sequence that has at least 90%, at least 95%, at least 98% or at least 99% (but less than 100%) sequence identity to any of the HCVR, LCVR, and/or CDR amino acid sequences disclosed herein. Sequence identity may be measured by methods known in the art (e.g., GAP, BESTFIT, and BLAST). Antibodies and antigen-binding fragments having an HCVR, LCVR, and/or CDR amino acid sequence with less than 100% identity to a corresponding sequence disclosed herein may have one or more substitutions of amino acids with other amino acids having similar characteristics (e.g., charge, side-chain size, hydrophobicity/hydrophilicity, backbone conformation and rigidity, etc.). Such changes can frequently be made without significantly disrupting the biological activity of the antibody or fragment.

Those of skill in the art recognize that, in general, limited (e.g., single) amino acid substitutions in non-essential regions of a polypeptide do not substantially alter biological activity (see, e.g., Watson et al. (1987) MOLECULAR BIOLOGY OF THE GENE, The Benjamin/Cummings Pub. Co., p. 224 (4^{th} Ed.)). Also, substitutions of structurally or functionally similar amino acids are less likely to significantly disrupt biological activity. Examples of groups of amino acids that have side chains with similar chemical properties include 1) aliphatic side chains: glycine, alanine, valine, leucine and isoleucine; 2) aliphatic-hydroxyl side chains: serine and threonine; 3) amide-containing side chains: asparagine and glutamine; 4) aromatic side chains: phenylalanine, tyrosine, and tryptophan; 5) basic side chains: lysine, arginine, and histidine; 6) acidic side chains: aspartate and glutamate, and 7) sulfur-containing side chains: cysteine and methionine.

### Species Selectivity and Species Cross-Reactivity

The present disclosure, according to certain embodiments, provides antigen-binding molecules that bind to human 4-1BB but not to 4-1BB from other species. The present disclosure also provides antigen-binding molecules that bind to human CD22 but not to CD22 from other species. The present disclosure also includes antigen-binding molecules that bind to human CD22 and to CD22 from one or more non-human species and/or antigen-binding molecules that bind to human 4-1BB and to 4-1BB from one or more non-human species.

According to some embodiments, antigen-binding molecules are provided which bind to human CD22 and human 4-1BB and may bind or not bind, as the case may be, to one or more of mouse, rat, guinea pig, hamster, gerbil, pig, cat, dog, rabbit, goat, sheep, cow, horse, camel, cynomolgus, marmoset, rhesus or chimpanzee CD22 and/or 4-1BB. For example, in an exemplary embodiment, bispecific antigen-binding molecules are provided comprising a first antigen-binding domain that binds human CD22 and cynomolgus CD22, and a second antigen-binding domain that specifically binds human 4-1BB.

### Immunoconjugates

The disclosure encompasses CD22×4-1BB antigen-binding proteins, e.g., antibodies or antigen-binding fragments, such as REGN9220, REGN9289, and REGN17596, conjugated to another moiety, e.g., a therapeutic moiety (an “immunoconjugate”). In an embodiment, an CD22×4-1BB antigen-binding protein, e.g., antibody or antigen-binding fragment, is conjugated to any of the further therapeutic agents set forth herein. As used herein, the term “immunoconjugate” refers to an antigen-binding protein, e.g., an antibody or antigen-binding fragment, which is chemically or biologically linked to another antigen-binding protein, a drug, a radioactive agent, a reporter moiety, an enzyme, a peptide, a protein or a therapeutic agent.

In certain embodiments, the therapeutic moiety may be a cytotoxin, a chemotherapeutic drug, an immunosuppressant or a radioisotope. Cytotoxic agents include any agent that is detrimental to cells. Examples of suitable cytotoxic agents and chemotherapeutic agents for forming immunoconjugates are known in the art. See, e.g., WO 05/103081.

### Therapeutic Uses of the Antigen-Binding Molecules

The multispecific antibodies and antigen-binding molecules of the disclosure (and therapeutic compositions comprising the same) are useful, inter alia, for treating any disease or disorder in which stimulation, activation and/or targeting of an immune response would be beneficial. In particular, the CD22×4-1BB multispecific antigen-binding molecules of the present disclosure may be used for the treatment, prevention and/or amelioration of a hyperproliferative disease, for example, cancer. in certain embodiments, the present disclosure provides methods for treating cancer in a subject, comprising administering a therapeutically effective dose of CD22×4-1BB antigen-binding protein, e.g., REGN9220, REGN9289, or REGN17596 to the subject.

A hyperproliferative disease, for the purposes herein, refers to a disease characterized by abnormal, excessive and/or uncontrolled cell growth, e.g., a cancer wherein the cells express CD22. For example, hyperproliferative diseases include cancers. Exemplary cancers include, but are not limited to anal cancer, angiosarcoma, a B cell cancer, basal cell carcinoma, bladder cancer, bone cancer, brain cancer, breast cancer, cervical cancer, cholangiocarcinoma, chondrosarcoma, chronic lymphocytic leukemia colon cancer, colorectal cancer, cutaneous squamous cell carcinoma, endometrial cancer, esophageal cancer, glioblastoma multiforme, head and neck squamous cell cancer, hepatocellular carcinoma, kidney cancer, liver cancer, lung cancer, lymphoma, leukemia, Merkel cell carcinoma, melanoma, myeloma, non-small cell lung cancer, ovarian cancer, pancreatic cancer, prostate cancer, salivary gland cancer, skin cancer, soft tissue sarcoma, stomach cancer, a T cell cancer, testicular cancer, and uterine cancer.

Accordingly, the antibodies and the multispecific antigen-binding molecules of the present disclosure can be used in treating a wide range of cancers.

Cancer characterized by solid tumor cells or cancerous blood cells may be an CD22-expressing cancer e.g., wherein CD22 expression in the cells of the particular subject to be treated has been confirmed, includes a B cell cancer, esophageal carcinoma, lung squamous cell carcinoma, lung adenocarcinoma, cervical squamous cell carcinoma, endometrial adenocarcinoma, bladder urothelial carcinoma, lung cancer (e.g., non-small cell lung cancer), colorectal cancer, rectal cancer, endometrial cancer, skin cancer (e.g., head & neck squamous cell carcinoma), brain cancer (e.g., glioblastoma multiforme), lymphoma, leukemia, breast cancer, gastroesophageal cancer, (e.g., gastroesophageal adenocarcinoma), prostate cancer, ovarian cancer, melanoma, basal cell carcinoma, cervical cancer, diffuse large B cell lymphoma, and/or multiple myeloma.

The antigen-binding molecules of the present disclosure may also be used to treat, e.g., primary and/or metastatic tumors arising in the blood, lymph, bone, colon, lung, breast, ovary, kidney, and bladder (or from any cancer discussed herein).

The antigen-binding proteins of the present disclosure may also be used to treat residual cancer in a subject. As used herein, the term “residual cancer” means the existence or persistence of one or more cancerous cells in a subject following treatment with an anti-cancer therapy.

As used herein, the term “subject” refers to a mammal (e.g., rat, mouse, cat, dog, cow, sheep, horse, goat, rabbit), preferably a human, for example, in need of prevention and/or treatment of cancer. The subject may have a cancer, be predisposed to developing such a condition, and/or would benefit from administration of a bispecific antibody or antigen-binding fragment thereof of the present disclosure. In one embodiment, the subject may have, or be at risk of developing, a hyperproliferative disease.

Methods for treating or preventing a cancer (e.g., a CD22-expressing cancer) in a subject in need of said treatment or prevention by administering a therapeutically effective dose amount CD22×4-1BB antigen-binding protein, in association with an additional therapeutic agent are part of the present disclosure. Additional therapeutic agents are disclosed elsewhere herein.

An “effective” or “therapeutically effective” amount of CD22×4-1BB antigen-binding protein, e.g., antibody or antigen-binding fragment, for treating or preventing a hyperproliferative disease, such as cancer, is the amount of the antigen-binding protein sufficient to alleviate one or more signs and/or symptoms of the disease in the treated subject, whether by inducing the regression or elimination of such signs and/or symptoms or by inhibiting the progression of such signs and/or symptoms. In some embodiments, a therapeutically effective dose of CD22×4-1BB antigen-binding protein is 0.1-2000 mg. The dose amount may vary depending upon the age and the size of a subject to be administered, target disease, conditions, route of administration, and the like. In certain embodiments, the initial dose may be followed by administration of a second or a plurality of subsequent doses of antigen-binding protein in an amount that can be approximately the same or less or more than that of the initial dose, wherein the subsequent doses may be separated by 1-8 weeks.

The dose of antigen-binding molecule administered to a subject may vary depending upon the age and the size of the subject, target disease, conditions, route of administration, and the like. In one embodiment, the dose is typically calculated according to body weight or body surface area. In other embodiments, the dose is a flat dose (for example, in milligrams), administered irrespective of the body weight. Depending on the severity of the condition, the frequency and the duration of the treatment can be adjusted.

### Combination Therapies

The bispecific antigen-binding molecules of the present disclosure may be used in combination with one or more agents, for example, in treating a cancer in a subject. In certain embodiments, the bispecific antigen-binding molecules may be administered in combination with one or more agents, for example, a corticosteroid, to reduce or ameliorate one or more adverse side effects, e.g., cytokine storm. In certain embodiments, the bispecific antigen-binding molecules may be administered in combination with one or more therapeutic agents or therapies for enhanced efficacy in treating cancer. Exemplary additional therapeutic agents or therapies that may be combined with or administered in combination with an antigen-binding molecule of the present disclosure include, e.g., chemotherapy (e.g., anti-cancer chemotherapy, for example, paclitaxel, docetaxel, vincristine, cisplatin, carboplatin or oxaliplatin), radiation therapy, surgery, a checkpoint inhibitor, a PD-1 inhibitor (e.g., an anti-PD-1 antibody such as pembrolizumab, nivolumab, or cemiplimab), a CTLA-4 inhibitor, LAG3 inhibitor, TIM3 inhibitor, a GITR agonist, OX40 agonist, 4-1BB agonist, an oncolytic virus, a cancer vaccine, a CAR-T cell, a nucleic acid therapeutic, a stem cell transplant, a modified IL2, modified IL12, IL15, IL6 inhibitor (e.g., sarilumab or tocilizumab), IL4R inhibitor (e.g., dupilumab), EGFR inhibitor, Ang2 inhibitor, VEGF inhibitor, a corticosteroid, a bispecific antibody or antigen-binding fragment thereof that binds CD3 and a tumor associated antigen (TAA) (e.g., CD20, or CD19).

The additional agents may be administered just prior to, concurrent with, or shortly after the administration of an antigen-binding molecule of the present disclosure; (for purposes of the present disclosure, such an administration regimen is considered the administration of an antigen-binding molecule “in combination with” an additional agent or therapeutic agent or therapy).

### Pharmaceutical Formulations and Administration

The present disclosure provides compositions that include CD22×4-1BB antigen-binding proteins and one or more ingredients; as well as methods of use thereof and methods of making such compositions. Pharmaceutical formulations (e.g., aqueous pharmaceutical formulations that include water) comprising an CD22×4-1BB antigen-binding protein of the present disclosure and a pharmaceutically acceptable carrier or excipient are part of the present disclosure.

The pharmaceutical compositions of the disclosure can be formulated with suitable carriers, excipients, and other agents that provide improved transfer, delivery, tolerance, and the like. A multitude of appropriate formulations can be found in the formulary known to all pharmaceutical chemists: Remington's Pharmaceutical Sciences, Mack Publishing Company, Easton, PA. These formulations include, for example, powders, pastes, ointments, jellies, waxes, oils, lipids, lipid (cationic or anionic) containing vesicles (such as LIPOFECTIN™, Life Technologies, Carlsbad, CA), DNA conjugates, anhydrous absorption pastes, oil-in-water and water-in-oil emulsions, emulsions carbowax (polyethylene glycols of various molecular weights), semi-solid gels, and semi-solid mixtures containing carbowax. See also Powell et al. “Compendium of excipients for parenteral formulations” PDA (1998) J Pharm Sci Technol 52:238-311.

To prepare pharmaceutical formulations of the CD22×4-1BB antigen-binding proteins, e.g., antibodies and antigen-binding fragments thereof (e.g., REGN9220, REGN9289, or REGN17596), the antigen-binding protein is admixed with a pharmaceutically acceptable carrier or excipient. See, e.g., Remington's Pharmaceutical Sciences and U.S. Pharmacopeia: National Formulary, Mack Publishing Company, Easton, Pa. (1984); Hardman, et al. (2001) Goodman and Gilman's The Pharmacological Basis of Therapeutics, McGraw-Hill, New York, N.Y.; Gennaro (2000) Remington: The Science and Practice of Pharmacy, Lippincott, Williams, and Wilkins, New York, N.Y.; Avis, et al. (eds.) (1993) Pharmaceutical Dosage Forms: Parenteral Medications, Marcel Dekker, NY; Lieberman, et al. (eds.) (1990) Pharmaceutical Dosage Forms: Tablets, Marcel Dekker, NY; Lieberman, et al. (eds.) (1990) Pharmaceutical Dosage Forms: Disperse Systems, Marcel Dekker, NY; Weiner and Kotkoskie (2000) Excipient Toxicity and Safety, Marcel Dekker, Inc., New York, N.Y. In some embodiments, the pharmaceutical formulation is sterile. Such compositions are part of the present disclosure.

Pharmaceutical formulations of the present disclosure include an CD22×4-1BB antigen-binding protein and a pharmaceutically acceptable carrier including, for example, water, buffering agents, preservatives and/or detergents.

The scope of the present disclosure includes desiccated, e.g., freeze-dried compositions, comprising an CD22×4-1BB antigen-binding protein, e.g., antibody or antigen-binding fragment thereof, or a pharmaceutical formulation thereof that includes a pharmaceutically acceptable carrier but substantially lacks water.

Various delivery systems are known and can be used to administer the pharmaceutical composition of the disclosure, e.g., encapsulation in liposomes, microparticles, microcapsules, recombinant cells capable of expressing the mutant viruses, receptor mediated endocytosis (see, e.g., Wu et al., 1987, J. Biol. Chem. 262:4429-4432). Methods of introduction include, but are not limited to, intradermal, intramuscular, intraperitoneal, intravenous, subcutaneous, intranasal, rectal, intestinal, epidural, and oral routes. The composition may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local.

As discussed herein, the present disclosure provides a vessel (e.g., a plastic or glass vial) or injection device (e.g., syringe, pre-filled syringe or autoinjector) comprising any of the CD22×4-1BB antigen-binding proteins herein, e.g., antibodies or antigen-binding fragments thereof, or a pharmaceutical formulation comprising a pharmaceutically acceptable carrier or excipient thereof.

A pharmaceutical composition of the present disclosure can be delivered subcutaneously or intravenously with a standard needle and syringe. With respect to subcutaneous delivery, a pen delivery device, as known in the art, may be used in delivering a pharmaceutical composition of the present disclosure. Such a pen delivery device can be reusable or disposable.

Numerous reusable and disposable pens and autoinjector delivery devices have applications in the subcutaneous delivery of a pharmaceutical composition of the present disclosure. See e.g., AUTOPEN™ (Owen Mumford, Inc., Woodstock, UK) or the HUMIRA™ Pen (Abbott Labs, Abbott Park, IL).

In certain situations, the pharmaceutical composition can be delivered in a controlled release system. In one embodiment, a pump may be used (see Langer, supra; Sefton, 1987, CRC Crit. Ref. Biomed. Eng. 14:201). In another embodiment, polymeric materials can be used; see, Medical Applications of Controlled Release, Langer and Wise (eds.), 1974, CRC Pres., Boca Raton, Florida. In yet another embodiment, a controlled release system can be placed in proximity of the composition's target, thus requiring only a fraction of the systemic dose (see, e.g., Goodson, 1984, in Medical Applications of Controlled Release, supra, vol. 2, pp. 115-138). Other controlled release systems are discussed in the review by Langer, 1990, Science 249:1527-1533.

The injectable preparations may include dosage forms for intravenous, subcutaneous, intracutaneous and intramuscular injections, drip infusions, etc. These injectable preparations may be prepared by methods publicly known. For example, the injectable preparations may be prepared, e.g., by dissolving, suspending or emulsifying the antibody or its salt described above in a sterile aqueous medium or an oily medium conventionally used for injections. As the aqueous medium for injections, there are, for example, physiological saline and other isotonic solutions which may be used in combination with an appropriate solubilizing agent. Injectable oily mediums are also part of the present disclosure. Such oily mediums may be combined with a solubilizing agent.

Advantageously, the pharmaceutical compositions for oral or parenteral use described above are prepared into dosage forms in a unit dose suited to fit a dose of the active ingredients. Such dosage forms in a unit dose include, for example, tablets, pills, capsules, injections (ampoules), suppositories, etc. The amount of the aforesaid antibody contained is generally about 0.1 to about 2000 mg per dosage form in a unit dose; especially in the form of injection.

The present disclosure also provides kits comprising an isolated antibody or antigen-binding fragment thereof that comprises an antigen-binding domain that binds specifically to human CD22 in a tumor cell, optionally wherein the antibody or antigen-binding fragment thereof further comprises an antigen-binding domain that binds specifically to human 4-1BB for therapeutic uses as described herein. Kits typically include a label indicating the intended use of the contents of the kit and instructions for use. As used herein, the term “label” includes any writing, or recorded material supplied on, in or with the kit, or which otherwise accompanies the kit. Accordingly, this disclosure provides a kit for inhibiting a tumor in a subject in need thereof, the kit comprising: (a) a therapeutically effective dosage of the isolated antibody or antigen-binding fragment thereof; and (b) instructions for using the isolated antibody or antigen-binding fragment thereof in any of the methods disclosed herein.

### Diagnostic Uses

The bispecific antibodies of the present disclosure may also be used to detect and/or measure CD22 or 4-1BB, or CD22-expressing or 4-1BB-expressing cells in a sample, e.g., for diagnostic purposes. For example, CD22×4-1BB antibody or antigen-binding fragment thereof, may be used to diagnose a condition or disease characterized by aberrant expression (e.g., over-expression, under-expression, lack of expression, etc.) of CD22 or 4-1BB. Exemplary diagnostic assays for CD22 or 4-1BB may comprise, e.g., contacting a sample, obtained from a subject, with an antibody of the disclosure, wherein the antibody is labeled with a detectable label or reporter molecule. Alternatively, an unlabeled antibody can be used in diagnostic applications in combination with a secondary antibody which is itself detectably labeled. The detectable label or reporter molecule can be a radioisotope, such as ³H, ¹⁴C, ³²P, ³⁵S, or ¹²⁵I; a fluorescent or chemiluminescent moiety such as fluorescein isothiocyanate, or rhodamine; or an enzyme such as alkaline phosphatase, betagalactosidase, horseradish peroxidase, or luciferase. Specific exemplary assays that can be used to detect or measure CD22 or 4-1BB in a sample include enzyme-linked immunosorbent assay (ELISA), radioimmunoassay (RIA), and fluorescence-activated cell sorting (FACS). Samples that can be used in CD22 or 4-1BB diagnostic assays according to the present disclosure include any tissue or fluid sample obtainable from a subject which contains detectable quantities of CD22 or 4-1BB protein, or fragments thereof, under normal or pathological conditions. Generally, levels of CD22 or 4-1BB in a particular sample obtained from a healthy subject (e.g., a subject not afflicted with a disease or condition associated with abnormal CD22 or 4-1BB levels or activity) will be measured to initially establish a baseline, or standard, level of CD22 or 4-1BB. This baseline level of CD22 or 4-1BB can then be compared against the levels of CD22 or 4-1BB measured in samples obtained from individuals suspected of having a CD22 or 4-1BB related disease or condition.

### EXAMPLES

The following examples are put forth so as to provide those of ordinary skill in the art with a complete disclosure and description of how to make and use the technology of the present disclosure and are not intended to limit the scope of what the inventors regard as their invention. Likewise, the disclosure is not limited to any particular preferred embodiments described herein. Indeed, modifications and variations of the embodiments may be apparent to those skilled in the art upon reading this specification and can be made without departing from its spirit and scope. Efforts have been made to ensure accuracy with respect to numbers used (e.g., amounts, temperature, etc.) but some experimental errors and deviations should be accounted for. Unless indicated otherwise, parts are parts by weight, molecular weight is average molecular weight, temperature is in degrees Centigrade, room temperature is about 25° C., and pressure is at or near atmospheric.

### Example 1: Construction of CD22×4-1BB Multispecific Antibodies

This example relates to the construction of multispecific anti-CD22×anti-4-1BB 1+2 format antibodies, “multispecific CD22×4-1BB antibodies”. The multispecific antibodies created in accordance with the present example comprise three separate antigen-binding domains. The first antigen-binding arm comprises an antigen-binding domain that comprises a heavy chain variable region (HCVR) derived from a “parental” CD22 antibody (“VH-1”). The second antigen-binding arm comprises a second antigen-binding domain that comprises a HCVR derived from a parental anti-4-1BB antibody (“VH-2”) and a third antigen-binding domain that comprises a HCVR derived from a parental anti-4-1BB antibody (“VH-3”). Both the CD22 binding domain and the anti-4-1BB binding domains share a common light chain. The multispecific format creates antigen-binding domains that specifically recognize CD22 (e.g., on tumor cells) and 4-1BB (e.g., on T cells).

### Generation of Anti-CD22 Antibodies

Parental, bivalent anti-CD22 antibodies were obtained by immunizing a genetically engineered mouse comprising DNA encoding human immunoglobulin heavy and universal light chain variable regions with a human CD22 antigen.

The antibodies were characterized and selected for desirable characteristics including affinity, selectivity, etc. The antibodies may have a desired constant region, for example, wild-type or modified hIgG1 or hIgG4 constant region. As will be appreciated by a person of skill in the art, an antibody with a particular constant region (e.g., modified hIgG1) may be converted to an antibody with a different constant region (e.g., modified hIgG4). While the constant region may vary according to specific use, high-affinity antigen-binding and target specificity characteristics reside in the variable region.

Exemplified antibodies were constructed using human IgG1 or human IgG4 isotype. In certain embodiments, the IgG4 Fc domain comprises 2 or more amino acid changes as disclosed in US 2010/0331527 or US 2014/0243504. In certain embodiments, the human IgG4 Fc comprises a serine to proline mutation in the hinge region (S108P) to promote dimer stabilization.

Table 1 sets forth the amino acid sequence identifiers of the heavy and light chain variable regions and CDRs of selected anti-CD22 antibodies of the disclosure. The corresponding nucleic acid sequence identifiers are set forth in Table 2.

TABLE 1
Amino Acid Sequence Identifiers for Selected
Parental CD22 Monoclonal Antibodies

| Antibody | SEQ ID NOs: | | | | | | | |
|---|---|---|---|---|---|---|---|---|
| Designation | HCVR | HCDR1 | HCDR2 | HCDR3 | LCVR | LCDR1 | LCDR2 | LCDR3 |
| mAb33037P | 2 | 4 | 6 | 8 | 18 | 20 | GAS | 24 |
| mAb33041P | 32 | 34 | 36 | 38 | 18 | 20 | GAS | 24 |

TABLE 2
Nucleic Acid Sequence Identifiers for Selected
Parental CD22 Monoclonal Antibodies

| Antibody | SEQ ID NOS: | | | | | | | |
|---|---|---|---|---|---|---|---|---|
| Designation | HCVR | HCDR1 | HCDR2 | HCDR3 | LCVR | LCDR1 | LCDR2 | LCDR3 |
| mAb33037P | 1 | 3 | 5 | 7 | 17 | 19 | ggggcaagt | 23 |
| mAb33041P | 31 | 33 | 35 | 37 | 17 | 19 | ggggcaagt | 23 |

### Generation of Anti-4-1BB Antibodies

Parental, bivalent anti-4-1BB antibodies were obtained by immunizing a genetically engineered mouse comprising DNA encoding human immunoglobulin heavy and universal light chain variable regions with human 4-1BB protein fused to the Fc portion of mouse IgG2a, or with DNA encoding 4-1BB.

The antibody immune response was monitored by a 4-1BB-specific immunoassay. When a desired immune response was achieved, anti-4-1BB antibodies were isolated directly from antigen-positive B cells, as described in U.S. Pat. No. 7,582,298.

The antibodies were characterized and selected for desirable characteristics including affinity, selectivity, etc. The antibodies may have a desired constant region, for example, wild-type or modified hIgG1 or hIgG4 constant region. As will be appreciated by a person of skill in the art, an antibody with a particular constant region (e.g., modified hIgG1) may be converted to an antibody with a different constant region (e.g., modified hIgG4). While the constant region may vary according to specific use, high-affinity antigen-binding and target specificity characteristics reside in the variable region.

Exemplified antibodies were constructed using human IgG1 or human IgG4 isotype. In certain embodiments, the IgG4 Fc domain comprises 2 or more amino acid changes as disclosed in US 2010/0331527 or US 2014/0243504. In certain embodiments, the human IgG4 Fc comprises a serine to proline mutation in the hinge region (S108P) to promote dimer stabilization.

Table 3 sets forth the amino acid sequence identifiers of the heavy and light chain variable regions and CDRs of selected anti-4-1BB antibodies of the disclosure. The corresponding nucleic acid sequence identifiers are set forth in Table 4.

TABLE 3
Amino Acid Sequence Identifiers for Selected
Parental 4-1BB Monoclonal Antibodies

| Antibody | SEQ ID NOs: | | | | | | | |
|---|---|---|---|---|---|---|---|---|
| Designation | HCVR | HCDR1 | HCDR2 | HCDR3 | LCVR | LCDR1 | LCDR2 | LCDR3 |
| mAb25921 | 42 | 44 | 46 | 48 | 18 | 20 | GAS | 24 |
| mAb25923 | 10 | 12 | 14 | 16 | 18 | 20 | GAS | 24 |

TABLE 4
Nucleic Acid Sequence Identifiers for Selected
Parental 4-1BB Antibodies

| Antibody | SEQ ID NOS: | | | | | | | |
|---|---|---|---|---|---|---|---|---|
| Designation | HCVR | HCDR1 | HCDR2 | HCDR3 | LCVR | LCDR1 | LCDR2 | LCDR3 |
| mAb25921 | 41 | 43 | 45 | 47 | 17 | 19 | ggggcaagt | 23 |
| mAb25923 | 9 | 11 | 13 | 15 | 17 | 19 | ggggcaagt | 23 |

Generation of Multispecific Antibodies that Bind CD22 and 4-1BB

The multispecific antigen-binding molecules that bind CD22 and 4-1BB are also referred to herein as “anti-CD22×anti-4-1BB 1+2” or “anti-CD22×anti-4-1BB multispecific molecules”, or “anti-CD22/anti-4-1BB 1+2”, or “anti-CD22/anti-4-1BB multispecific molecules”, or “CD22×4-1BB 1+2”, or “CD22×4-1BB multispecific molecules”. The anti-CD22 portion of the anti-CD22×anti-4-1BB multispecific molecule is useful for targeting tumor cells that express CD22, and the anti-4-1BB portion of the multispecific molecule is useful for activating immune cells (e.g., T cells).

An individual 4-1BB-binding Fab (i.e., a heavy chain variable region with a heavy chain C_{H}1 domain and a light chain) binding to 4-1BB epitope 1 (ep1) or epitope 2 (ep2) were fused to the N-terminus of a 4-1BB VH domain from an existing IgG-like multispecific antibody targeting both CD22 and 4-1BB.

Mammalian expression vectors for individual heavy chains were created by InFusion Cloning (Takara Bio USA Inc.) following protocols provided by Takara Bio USA Inc. A CD22 heavy chain variable region (VH-1) was cloned into a heavy chain expression plasmid (CH1-1_CH2_CH3). A 4-1BB heavy chain variable region (VH-3, “outer VH”) was fused to a CH1 domain (CH1-3, “outer CH1”) with a linker followed by another 4-1BB heavy chain variable region (VH-2, “inner VH”) and cloned into a heavy chain expression plasmid (CH1-2_CH2_CH3(*)) containing the star mutation (H435R, Y436F, EU numbering).

Recombinant CD22×4-1BB 1+2 N-Fab multispecific antigen-binding molecules (msABM) were produced in CHO cells after transfection with 3 expression plasmids (i) CD22 heavy chain plasmid, (ii) 4-1BB+4-1BB heavy chain star plasmid, and (iii) a universal light chain plasmid. Stably transfected CHO cells were used to produce the CD22×4-1BB 1+2 N-Fab msABM, which were subsequently purified as described previously (Smith et al., Sci Rep. 2015 Dec. 11:5:17943).

Table 5 summarizes the component parts of selected multispecific CD22×4-1BB antibodies of the disclosure. Tables 6 and 7 set forth the amino acid and nucleic acid identifiers, respectively, of the selected multispecific antibodies. Table 8 shows the the amino acid and nucleic acid identifiers of the full-length heavy chain and light chain sequences of the selected mulispecific antibodies.

TABLE 5
Summary of Component Parts of Selected
Multispecific CD22 × 4-1BB Antibodies

| | D1 | D2 “outer” | D3 “inner” | |
|---|---|---|---|---|
| Multispecific | Anti-CD22 | Anti-4-1BB | Anti-4-1BB | Common Light |
| Antibody | Antigen-Binding | Antigen-Binding | Antigen-Binding | Chain Variable |
| Designation | Domain | Domain | Domain | Region |
| bsAb9220 | mAb33037P | mAb25923 | mAb25923 | ULC3-20 |
| bsAb9289 | mAb33037P | mAb25921 | mAb25921 | ULC3-20 |
| bsAb17596 | mAb33041P | mAb25923 | mAb25923 | ULC3-20 |

TABLE 6
Amino Acid Sequence Identifiers of Selected
Multispecific CD22 × 4-1BB Antibodies

| | D1 (Fab1) | | | | D2 (Fab2) | | | |
|---|---|---|---|---|---|---|---|---|
| | Anti-CD22 | | | | “Outer” Anti-4-1BB | | | |
| Multispecific | Antigen-Binding Domain | | | | Antigen-Binding Domain | | | |
| Antibody | | D1- | D1- | D1- | | D2- | D2- | D2- |
| Designation | VH-1 | HCDR1 | HCDR2 | HCDR3 | VH-2 | HCDR1 | HCDR2 | HCDR3 |
| bsAb9220 | 2 | 4 | 6 | 8 | 10 | 12 | 14 | 16 |
| bsAb9289 | 2 | 4 | 6 | 8 | 42 | 44 | 46 | 48 |
| bsAb17596 | 32 | 34 | 36 | 38 | 10 | 12 | 14 | 16 |

| | D3 (Fab3) | | | | | | | |
|---|---|---|---|---|---|---|---|---|
| | “Inner” Anti-4-1BB | | | | | | | |
| Multispecific | Antigen-Binding Domain | | | | Common | | | |
| Antibody | | D3- | D3- | D3- | Light Chain Variable Region | | | |
| Designation | VH-3 | HCDR1 | HCDR2 | HCDR3 | LCVR | LCDR1 | LCDR2 | LCDR3 |
| bsAb9220 | 10 | 12 | 14 | 16 | 18 | 20 | GAS | 24 |
| bsAb9289 | 42 | 44 | 46 | 48 | 18 | 20 | GAS | 24 |
| bsAb17596 | 10 | 12 | 14 | 16 | 18 | 20 | GAS | 24 |

TABLE 7
Nucleic Acid Sequence Identifiers of Selected Multispecific CD22 × 4-1BB Antibodies

| | D1 (Fab1) | | | | D2 (Fab2) | | | |
|---|---|---|---|---|---|---|---|---|
| Multispecific | Anti-CD22 | | | | “Outer” Anti-4-1BB | | | |
| Antibody | Antigen-Binding Domain | | | | Antigen-Binding Domain | | | |
| Designation | VH-1 | D1-HCDR1 | D1-HCDR2 | D1-HCDR3 | VH-2 | D2-HCDR1 | D2-HCDR2 | D2-HCDR3 |
| bsAb9220 | 1 | 3 | 5 | 7 | 51 | 52 | 53 | 54 |
| bsAb9269 | 1 | 3 | 5 | 7 | 55 | 56 | 57 | 58 |
| bsAb17596 | 31 | 33 | 35 | 37 | 51 | 52 | 53 | 54 |

| | D3 (Fab3) | | | | | | | |
|---|---|---|---|---|---|---|---|---|
| Multispecific | “Inner” Anti-4-1BB | | | | Common | | | |
| Antibody | Antigen-Binding Domain | | | | Light Chain Variable Region | | | |
| Designation | VH-3 | D3-HCDR1 | D3-HCDR2 | D3-HCDR3 | LCVR | LCDR1 | LCDR2 | LCDR3 |
| bsAb9220 | 9 | 11 | 13 | 15 | 17 | 19 | ggggcaagt | 23 |
| bsAb9269 | 41 | 43 | 45 | 47 | 17 | 19 | ggggcaagt | 23 |
| bsAb17596 | 9 | 11 | 13 | 15 | 17 | 19 | ggggcaagt | 23 |

TABLE 8
Amino Acid and Nucleic Acid Sequence Identifiers of Heavy Chains
and Light Chains of Selected Multispecific CD22 × 4-1BB Antibodies

| | | Amino Acid SEQ ID NOs: | | | Nucleic Acid SEQ ID NOs: | | |
|---|---|---|---|---|---|---|---|
| Multispecific | | Anti- | Anti-4 | | Anti- | Anti-4- | |
| Antibody | Antibody | CD22 | 1BB | Common | CD22 | 1BB | Common |
| Designation | Identifier | HC | HC | LC | HC | HC | LC |
| bsAb9220 | REGN9220 | 26 | 28 | 30 | 25 | 27 | 29 |
| bsAb9289 | REGN9289 | 26 | 50 | 30 | 25 | 49 | 29 |
| bsAb17596 | REGN17596 | 40 | 28 | 30 | 39 | 27 | 29 |

TABLE 8
Amino Acid and Nucleic Acid Sequence Identifiers of Heavy Chains
and Light Chains of Selected Multispecific CD22x4-1BB Antibodies

| Multispecific | | Amino Acid SEQ ID NOs: | | | Nucleic Acid SEQ ID NOs: | | |
|---|---|---|---|---|---|---|---|
| Antibody | Antibody | Anti- | Anti-4- | Common | Anti- | Anti-4- | Common |
| Designation | Identifier | CD22 HC | 1BB HC | LC | CD22 HC | 1BB HC | LC |
| bsAb9220 | REGN9220 | 26 | 28 | 30 | 25 | 27 | 29 |
| bsAb9289 | REGN9289 | 26 | 50 | 30 | 25 | 49 | 29 |
| bsAb17596 | REGN17596 | 40 | 28 | 30 | 39 | 27 | 29 |

Additional multispecific antibodies comprising a HCVR from one parental CD22 antibody and two HCVRs from a single or two different parental 4-1BB antibodies may be made using the techniques described herein. The parental CD22 antibodies used to generate these additional anti-CD22×anti-4-1BB multispecific antibodies have HCVR sequences described above in Table 1. The 4-1BB parental antibodies used to generate these additional anti-CD22×anti-4-1BB multispecific antibodies have the amino acid sequences described above in Table 3.

The multispecific antibodies described in the following examples consist of antigen-binding arms that bind to human CD22 and human 4-1BB protein (see Biacore binding data below). Exemplified multispecific antibodies contain a modified (chimeric) IgG4 Fc domain as set forth in U.S. Pat. No. 9,359,437

Controls: In addition to isotype controls, the following controls were used in the Examples below: Comparator 1: a human bivalent monoclonal antibody against 4-1BB having VH/VL sequences of antibody “20H4.9” according to US 2014/0193422 (BMS); Control 1: a bispecific antibody (1+2) with one arm comprising two domains, each binding to 4-1BB (derived from parental antibody mAb25921) and the other arm binding to an unrelated antigen; Control 2: a bispecific antibody (1+2) with one arm comprising 2 domains, each binding to 4-1BB (derived from parental antibody mAb25923) and the other arm binding to an unrelated antigen; Control 3: a bispecific antibody with one arm binding to 4-1BB (derived from parental antibody mAb25923) and the other arm binding to an unrelated antigen. Control 4: a bispecific antibody with one arm binding to CD3 and the other arm binding to an unrelated antigen.

### Example 2: Strong Binding of Multispecific CD22×4-1BB Antibodies by Surface Plasmon Resonance

This example relates to in vitro studies demonstrating the binding characteristics of anti-CD22 parental antibodies and multispecific anti-CD22×anti-4-1BB 1+2 format antibodies, “CD22×4-1BB antibodies”, binding to monomeric human, cynomolgus, and murine CD22; monomeric human, cynomolgus, and murine 4-1BB; and dimeric human 4-1BB proteins in an antibody capture format at 25° C.

Experimental procedure: Equilibrium dissociation constants (K_{D} values) of CD22×4-1BB and anti-CD22 parental antibodies binding to human, cynomolgus, and murine CD22 and CD22×4-1BB binding to human, cynomolgus, and murine 4-1BB were determined using real-time surface plasmon resonance biosensor technology on a Biacore 4000 or 1S+ instrument. The monomeric human, cynomolgus, and murine CD22 were expressed with a C-terminal myc-myc-hexahistidine tag (hCD22.mmH; mfCD22(XP_005588899.1).mmH; mfCD22(EHH59463.1).mmH; and mCD22.mmH), monomeric human, cynomolgus, and murine 4-1BB expressed with a C-terminal myc-myc-hexahistidine tag (h4-1BB.mmH; mf4-1BB.mmH; and m4-1BB.mmH, respectively), and dimeric human 4-1BB expressed with a C-terminal murine Fc tag (h4-1BB.mFc). Briefly, the CM5 Biacore sensor surface was derivatized by amine coupling with a monoclonal mouse anti-human Fc monoclonal antibody. All Biacore binding studies were performed in a buffer composed of 10 mM HEPES pH 7.4, 150 mM NaCl, 3 mM EDTA, 0.05% v/v Surfactant P20 (HBS-EP running buffer). Different concentrations of hCD22.mmH, mfCD22(XP_005588899.1).mmH, or mfCD22(EHH59463.1).mmH ranging from 0.37 nM to 90 nM in 3-fold serial dilutions; mCD22.mmH ranging from 10 to 90 nM in 3-fold serial dilutions; h4-1BB.mmH, mf4-1BB.mmH, m4-1BB.mmH, or h4-1BB.mFc, ranging from 1.1 to 270 nM in 3-fold serial dilutions were injected over the captured anti-CD22×4-1BB 1+2 format or CD22 parental antibodies at a flow rate of 30 μL/minute. Antibody-ligand association was monitored for 5 minutes, and dissociation was monitored for 10 minutes. At the end of each cycle, the CD22×4-1BB or anti-CD22 parental antibody capture surface was regenerated using a 12 second injection of 20 mM phosphoric acid. All binding kinetics experiments were performed at 25° C.

Data analysis: The specific SPR-Biacore sensorgrams were obtained by a double referencing procedure. This was performed by first subtracting the signal of each injection over a reference surface (anti-hFc) from the signal over the experimental surface (anti-hFc-captured CD22×4-1BB or anti-CD22 parental antibodies) thereby removing contributions from refractive index changes. In addition, running buffer injections were performed to allow subtraction of the signal changes resulting from the dissociation of captured 1+2 format or parental antibodies from the coupled anti-hFc surface. Kinetic association (kₐ) and dissociation (k_{d}) rate constants were determined by fitting the real-time sensorgrams to a 1:1 binding model using Scrubber v2.0c or Cytiva Insight v5.0 curve fitting software. Binding dissociation equilibrium constants (K_{D}) and dissociative half-lives (t½) were calculated from the kinetic rate constants as:

$${K}_{D} ( M ) = \frac{kd}{ka} , and t 1 / 2 ( min ) = \frac{ln ( 2 )}{60 * kd}$$

Results: The multispecific anti-CD22×anti-4-1BB 1+2 format antibodies REGN9220, REGN9289, and REGN17596 display outstanding CD22 and 4-1BB binding characteristics. The kinetic and equilibrium binding parameters for human, cynomolgus (XP_0055888899.1), cynomolgus (EHH59463.1), and mouse CD22 are set forth in Tables 9 through 12, respectively. The kinetic and equilibrium binding parameters for monomeric human, cynomolgus, and mouse CD22 are set forth in Tables 13 through 15, respectively. The kinetic and equilibrium binding parameters for dimeric human 4-1BB kinetics are set forth in Table 16. All results were determined at 25° C.

TABLE 9
Kinetic and Equilibrium Binding Parameters of Human CD22 to Surface-captured
Multispecific CD22x4-1BB Antibodies or anti-CD22 Parental Antibodies

| Multispecific | mAb | hCD22.mmH | | | | |
|---|---|---|---|---|---|---|
| Antibody | Capture | Bound at | kₐ | k_{d} | K_{D} | t½ |
| Designation | (RU) | 90 nM (RU) | (1/Ms) | (1/s) | (M) | (min) |
| mAb33037P | 266.7 ± 4.0 | 162.3 | 1.46E+05 | 1.19E−03 | 8.17E−09 | 9.7 |
| REGN9220 | 252.0 ± 1.6 | 68.9 | 1.32E+05 | 1.20E−03 | 9.06E−09 | 9.7 |
| REGN9289 | 207.3 ± 6.2 | 51.2 | 1.66E+05 | 1.27E−03 | 7.64E−09 | 9.1 |
| mAb33041P | 282.0 ± 4.1 | 48.6 | 7.23E+04 | 2.87E−04 | 3.97E−09 | 40.3 |
| REGN17596 | 204.6 ± 1.7 | 11.9 | IC* | IC* | IC* | IC* |

*IC = Inconclusive

TABLE 10
Kinetic and Equilibrium Binding Parameters of Cynomolgus CD22 (XP_0055888899.1)
to Surface-captured Multispecific CD22x4-1BB or anti-CD22 Parental Antibodies

| Multispecific | mAb | mfCD22 | | | | |
|---|---|---|---|---|---|---|
| Antibody | Capture | (XP_005588899.1).mmH | kₐ | k_{d} | K_{D} | t½ |
| Designation | (RU) | Bound at 90 nM (RU) | (1/Ms) | (1/s) | (M) | (min) |
| mAb33037P | 264.6 ± 0.7 | 103.6 | 6.93E+04 | 2.65E−03 | 3.82E−08 | 4.4 |
| REGN9220 | 257.4 ± 1.1 | 55.4 | 8.91E+04 | 2.33E−03 | 2.62E−08 | 5.0 |
| REGN9289 | 214.4 ± 1.7 | 27.5 | 4.54E+04 | 2.28E−03 | 5.03E−08 | 5.1 |
| mAb33041P | 281.0 ± 0.5 | 42.3 | 1.53E+05 | 1.04E−03 | 6.75E−09 | 11.2 |
| REGN17596 | 210.1 ± 2.5 | 1.8 | NB* | NB* | NB* | NB* |

*NB = Non-binding

TABLE 11
Kinetic and Equilibrium Binding Parameters of Cynomolgus CD22 (EHH59463.1) to
Surface-captured Multispecific CD22x4-1BB or anti-CD22 Parental Antibodies

| Multispecific | mAb | mfCD22 | | | | |
|---|---|---|---|---|---|---|
| Antibody | Capture | (EHH59463.1).mmH | kₐ | k_{d} | K_{D} | t½ |
| Designation | (RU) | Bound at 90 nM (RU) | (1/Ms) | (1/s) | (M) | (min) |
| mAb33037P | 265.0 ± 0.6 | 121.7 | 6.82E+04 | 2.41E−03 | 3.53E−08 | 4.8 |
| REGN9220 | 256.4 ± 1.3 | 65.2 | 9.84E+04 | 2.18E−03 | 2.22E−08 | 5.3 |
| REGN9289 | 211.5 ± 1.8 | 33.1 | 6.11E+04 | 2.09E−03 | 3.42E−08 | 5.5 |
| mAb33041P | 280.8 ± 0.7 | 47.6 | 9.88E+04 | 8.73E−04 | 8.84E−09 | 13.2 |
| REGN17596 | 208.2 ± 2.0 | 3.1 | NB* | NB* | NB* | NB* |

*NB = Non-binding

TABLE 12
Lack of Murine CD22 Binding to Surface-captured Multispecific
CD22x4-1BB or anti-CD22 Parental Antibodies

| Multispecific | mAb | mCD22.mmH | | | | |
|---|---|---|---|---|---|---|
| Antibody | Capture | Bound at 90 nM | kₐ | k_{d} | K_{D} | t½ |
| Designation | (RU) | (RU) | (1/Ms) | (1/s) | (M) | (min) |
| mAb33037P | 187.3 ± 3.1 | −0.2 | NB* | NB* | NB* | NB* |
| REGN9220 | 217.5 ± 1.0 | −0.3 | NB* | NB* | NB* | NB* |
| REGN9289 | 276.5 ± 4.0 | −0.5 | NB* | NB* | NB* | NB* |
| mAb33041P | 239.9 ± 0.8 | −0.4 | NB* | NB* | NB* | NB* |
| REGN17596 | 215.6 ± 1.6 | −0.3 | NB* | NB* | NB* | NB* |

*NB = Non-binding

TABLE 13
Kinetic and Equilibrium Binding Parameters of Monomeric Human
4-1BB to Surface-captured Multispecific CD22x4-1BB Antibodies

| Multispecific | mAb | h4-1BB.mmH | | | | |
|---|---|---|---|---|---|---|
| Antibody | Capture | Bound at | kₐ | k_{d} | K_{D} | t½ |
| Designation | (RU) | 270 nM (RU) | (1/Ms) | (1/s) | (M) | (min) |
| REGN9220 | 261.2 ± 0.3 | 44.3 | 7.62E+04 | 5.01E−03 | 6.57E−08 | 2.3 |
| REGN9289 | 219.8 ± 0.8 | 33.9 | 2.96E+04 | 4.28E−04 | 1.45E−08 | 27.0 |
| REGN17596 | 212.6 ± 1.4 | 29.8 | 5.32E+04 | 5.57E−03 | 1.05E−07 | 2.1 |

TABLE 14
Kinetic and Equilibrium Binding Parameters of Cynomolgus 4-
1BB to Surface-captured Multispecific CD22x4-1BB Antibodies

| Multispecific | mAb | mf4-1BB.mmH | | | | |
|---|---|---|---|---|---|---|
| Antibody | Capture | Bound at 270 nM | kₐ | k_{d} | K_{D} | t½ |
| Designation | (RU) | (RU) | (1/Ms) | (1/s) | (M) | (min) |
| REGN9220 | 259.0 ± 0.8 | 61.9 | 1.36E+05 | 5.43E−03 | 4.01E−08 | 2.1 |
| REGN9289 | 222.4 ± 0.7 | 35.0 | 4.90E+04 | 9.00E−04 | 1.83E−08 | 12.8 |
| REGN17596 | 217.7 ± 0.9 | 36.7 | 8.14E+04 | 5.54E−03 | 6.81E−08 | 2.1 |

TABLE 15
Lack of Monomeric Murine 4-1BB Binding to Surface-
captured Multispecific CD22x4-1BB Antibodies

| Multispecific | mAb | m4-1BB.mmH | | | | |
|---|---|---|---|---|---|---|
| Antibody | Capture | Bound at | kₐ | k_{d} | K_{D} | t½ |
| Designation | (RU) | 270 nM (RU) | (1/Ms) | (1/s) | (M) | (min) |
| REGN9220 | 217.0 ± 0.5 | −0.5 | NB* | NB* | NB* | NB* |
| REGN9289 | 269.8 ± 4.6 | −0.6 | NB* | NB* | NB* | NB* |
| REGN17596 | 214.1 ± 1.4 | −0.3 | NB* | NB* | NB* | NB* |

*NB = Non-binding

TABLE 16
Kinetic and Equilibrium Binding Parameters of Dimeric Human
4-1BB to Surface-captured Multispecific CD22x4-1BB Antibodies

| Multispecific | mAb | m4-1BB.mmH | | | | |
|---|---|---|---|---|---|---|
| Antibody | Capture | Bound at | kₐ | k_{d} | K_{D} | t½ |
| Designation | (RU) | 270 nM (RU) | (1/Ms) | (1/s) | (M) | (min) |
| REGN9220 | 260.5 ± 1.3 | 112.4 | 2.62E+05 | 3.51E−04 | 1.34E−09 | 33.0 |
| REGN9289 | 220.5 ± 1.7 | 99.6 | 1.22E+05 | 6.60E−05 | 5.43E−10 | 175.0 |
| REGN17596 | 213.8 ± 3.0 | 107.1 | 2.80E+05 | 1.01E−04 | 3.59E−10 | 114.8 |

### Example 3: Strong Binding of Multispecific CD22×4-1BB Antibody to Cells Engineered to Express CD22 or 4-1BB and a CD22+ Cancer Cell Line

This example relates to an in vitro study demonstrating the ability of a multispecific CD22×4-1BB antibody to bind to cell lines engineered to express human CD22 or human 4-1BB and the CD22+ cancer cell line Ramos.2G6.4C10 as measured by flow cytometry.

The following cell lines were used in this study: Jurkat/NFkB-Luc/h4-1BB: Jurkat cells (which are CD22 negative) stably transduced with a nuclear factor kappa-light-chain-enhancer of activated B cells (NF-κB)-luciferase reporter construct were engineered to express human 4-1BB (amino acids M1-L255 of accession number NM_001561); HEK293/pRG984-hCD20: a cell line made by stably transducing HEK293 cells (which are CD22 negative), a human embryonic kidney cell line isolated from a fetus, with human CD20 (Uniprot accession #: P11836, amino acids M1 to P297); HEK293/hCD19/hCD20/hCD22 sorted: a cell line made by stably transducing HEK293/pRG984-hCD20 cells with human CD19 (amino acids M1 to R556 of accession number NP_001761.3) and human CD22 (amino acids D20 to H846 of accession number NP_001762.2); Ramos.2G6.4C10: a B lymphocyte cell line (which endogenously expresses human CD22) that was isolated from a 3-year-old, male patient with Burkitt's Lymphoma. The following antibodies, as described in Example 1, were used in this study: REGN9220, REGN9289, REGN17596, Comparator 1, Control 1, Control 2, mAb33037P, mAb33041P, and Isotype control.

Experimental procedure: HEK293/hCD20+/−CD22 cells were lifted with trypsin, washed and resuspended in stain buffer (1% FBS in PBS). HEK293, Ramos and Jurkat/NFkB-Luc/h41BB cells were filtered through a 0.4 μm Greiner cell strainer and then counted. Cells were added to 384-well V-bottom plates at 40,000 cells/well. Antibodies were titrated in stain buffer from 100 nM to 1.7 pM in a 12-point 1:3 dose titration, with a final point containing stain buffer only, and added to cells. Cells and antibodies were incubated for 30 min at 4° C. and then washed in stain buffer. Cells were resuspended in a mix of 2 μg/ml allophycocyanin (APC) conjugated goat-anti human secondary antibody and viability dye (reconstituted in DMSO according to the manufaturer's protocol and diluted 1:1000 in PBS) and incubated for 30 min on ice and then washed in stain buffer. Cells were then resuspended in 2% PFA for 30 min at 4° C., washed, then analyzed by flow cytometry. EC₅₀ values of the antibodies were determined from a 4 parameter logistic equation over a 12-point dose response curve (including secondary only control) using GraphPad Prism software.

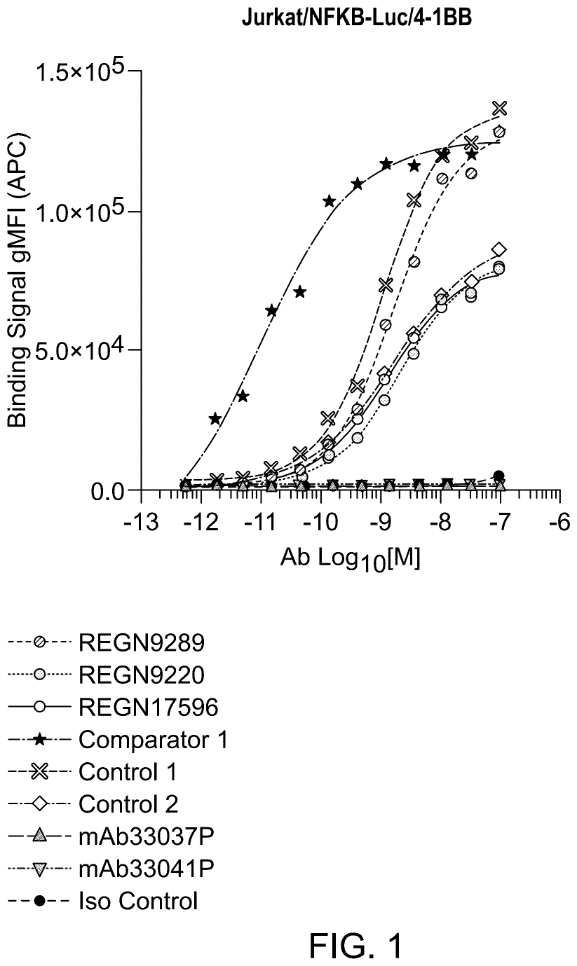

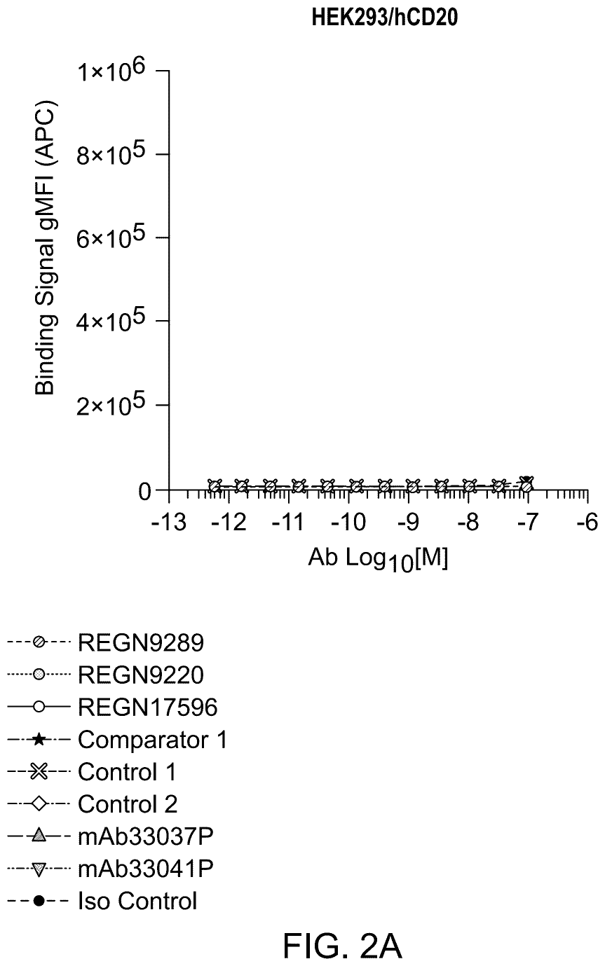

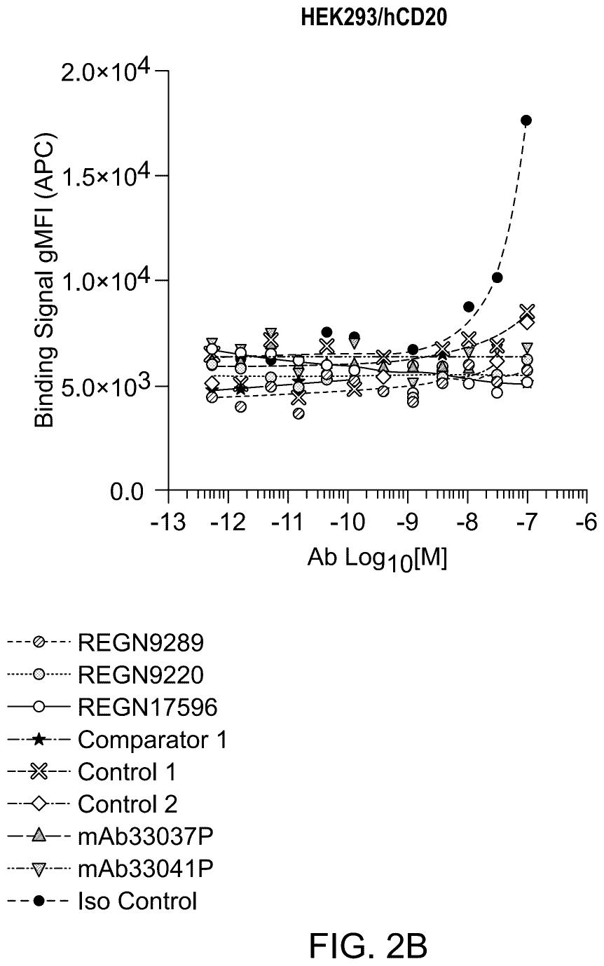

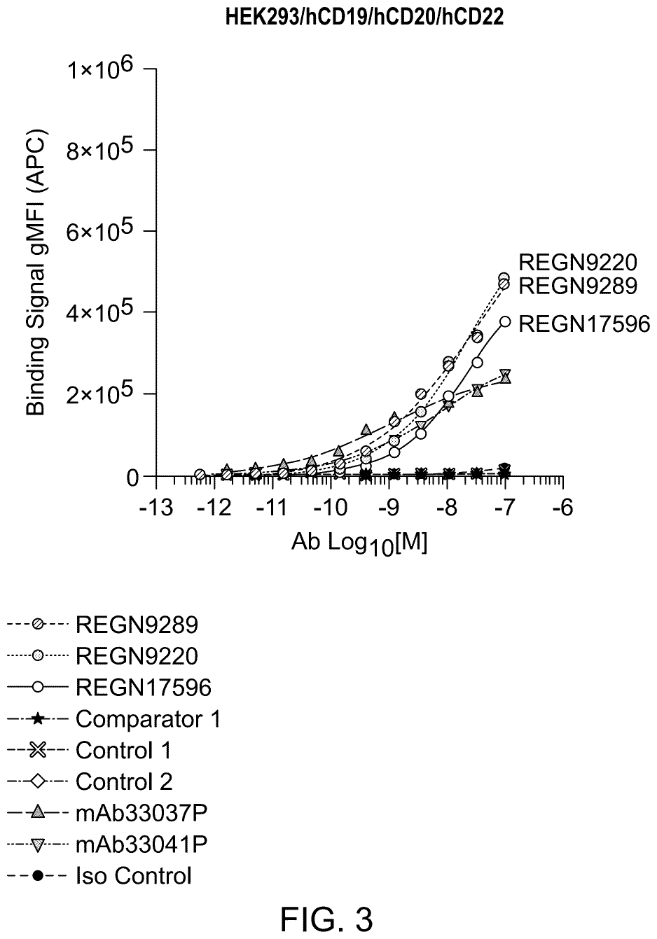

Results: The multispecific CD22×4-1BB antibodies REGN9220, REGN9289, and REGN17596 specifically bound to the surface of cells engineered to express human CD22 or 4-1BB and cells of the CD22+ Ramos B lymphocyte cancer cell line with high affinity (FIGS. 1-4). Table 17 sets forth the EC₅₀ values for selected multispecific CD22×4-1BB antibodies and control antibodies binding to each cell type.

TABLE 17
Binding Affinities of Antibodies to Cells Engineered to
Express CD22 or 4-1BB and to a CD22+ Cancer Cell Line

| | | | HEK293/hCD19/ | | | | Jurkat/NFkB- | |
|---|---|---|---|---|---|---|---|---|
| | Ramos | | hCD20/hCD22 | | HEK293/hCD20 | | Luc/h4-1BB | |
| | | Max | | Max | | Max | | Max |
| | EC₅₀ | signal | EC₅₀ | signal | EC₅₀ | signal | EC₅₀ | signal |
| Antibody | (M) | (gMFI) | (M) | (gMFI) | (M) | (gMFI) | (M) | (gMFI) |
| REGN9220 | 7.1E−09 | 6.7E+05 | NC | 4.9E+05 | ND | 6.1E+03 | 2.2E−09 | 8.0E+04 |
| REGN9289 | 4.5E−09 | 6.4E+05 | NC | 4.7E+05 | ND | 5.8E+03 | 1.8E−09 | 1.3E+05 |
| REGN17596 | 9.6E−09 | 7.8E+05 | NC | 3.8E+05 | ND | 6.8E+03 | 1.4E−09 | 8.0E+04 |
| Comparator 1 | ND | 1.5E+04 | ND | 5.1E+03 | ND | 6.6E+03 | 1.0E−11 | 1.3E+05 |
| Control 1 | ND | 1.5E+04 | ND | 8.0E+03 | ND | 8.5E+03 | 1.1E−09 | 1.4E+05 |
| Control 2 | ND | 1.5E+04 | ND | 7.3E+03 | ND | 8.0E+03 | 1.8E−09 | 8.6E+04 |
| mAb33037P | 2.9E−10 | 4.7E+05 | 1.1E−09 | 2.4E+05 | ND | 6.8E+03 | ND | 1.3E+03 |
| mAb33041P | 1.2E−09 | 4.3E+05 | 8.8E−09 | 2.5E+05 | ND | 7.5E+03 | ND | 1.8E+03 |
| Isotype control | ND | 1.6E+04 | ND | 2.1E+04 | ND | 1.8E+04 | ND | 4.9E+03 |

NC: Not calculated because the data did not fit a 4-parameter logistic equation
ND: Not determined because a concentration-dependent response was not observed

### Example 4: Potent Ability of Multispecific CD22×4-1BB Antibodies to Activate a T Cell Line Expressing 4-1BB

This example relates to an in vitro study demonstrating the ability of multispecific CD22×4-1BB antibodies to specifically activate the 4-1BB receptor in a 4-1BB+ T cell line in the presence of target cells expressing CD22 in en engineered reporter assay.

In this assay, engineered Jurkat cells express the reporter gene luciferase under the control of the transcription factor NF-κB (NFκB-Luc) along with the costimulatory receptor 4-1BB (Jurkat/NFκB-Luc/h4-1BB). The target cells used in this assay were HEK293 cells engineered to express CD20 alone (HEK293/hCD20) or in combination with CD22 (HEK293/hCD19/hCD20/hCD22); or Ramos cells that endogenously express CD22 and CD20. The ability of 4-1BB antibodies to stimulate 4-1BB activity is assessed by combining reporter cells with target cells and a titration of antibody. Activation of 4-1BB results in NFκB-driven luciferase production, which is then measured via a readout of luminescence.

The following target cells were tested in this study: HEK293/pRG984-hCD20: a cell line made by stably transducing HEK293 cells, a human embryonic kidney cell line isolated from a fetus, with human CD20 (Uniprot accession #: P11836, amino acids M1 to P297); HEK293/hCD19/hCD20/hCD22 sorted: a cell line made by stably transducing HEK293/pRG984-hCD20 cells with human CD19 (amino acids M1 to R556 of accession number NP_001761.3) and human CD22 (amino acids D20 to H846 of accession number NP_001762.2); and Ramos.2G6.4C10: a B lymphocyte cell line that was isolated from a 3-year-old, male patient with Burkitt's Lymphoma. The following antibodies, as described in Example 1, were used in this study: REGN9220, REGN9289, REGN17596, Comparator 1, Control 1, Control 2, mAb33037P, mAb33041P, and Isotype control.

Experimental Procedure: One day before the experiment, Jurkat/NFκB-luc reporter cells were split to 7.5×10⁵ cells/mL in growth medium. On the day of the experiment the cells were resuspended in assay medium and added to 384-well white plates at 1.5×10⁴ cells/well. Target cells were then added to wells at 4.0×10³ cells/well. Multispecific CD22×4-1BB and control antibodies were prepared in assay medium and titrated from 50 nM to 847 fM in a 1:3 dilution, the final point of the 12-point dilution containing no titrated antibody, and added, in duplicate, to the appropriate wells. Plates were incubated at 37° C. and 5% CO₂ for 5 hours and then ONE-Glo™ luciferase substrate was added to each well according to manufacturer's instructions. The luciferase activity was recorded as a luminescence signal using a multilable plate reader and expressed as relative light units (RLU).

The EC₅₀ values were determined by a 4-parameter logistic equation over a 12-point response curve using GraphPad Prism™ software. Signal recorded for the 12^{th} point on the dilution curve (no titrated antibody) was plotted at 282 fM. Maximal RLU is given as the mean max response detected within the tested dose range.

Results: In the presence of Jurkat/NFkB-Luc/4-1BB cells and target cells expressing CD22, the addition of the multispecific CD22×41BB antibodies REGN9220, REGN9289 and REGN17596 led to a dose dependent increase in NF-κB activity. In the absence of CD22 expression on target cells, only Comparator 1 led to dose dependent increase in NF-kB activity. Table 18 sets forth potency values of selected multispecific CD22×4-1BB antibodies and control antibodies in the presence of specific cell types.

TABLE 18
Potency values, EC₅₀ and Maximim Reporter Activity, of Antibodies

| | | | HEK293/hCD19/ | | | |
|---|---|---|---|---|---|---|
| | HEK293/hCD20 | | hCD20/hCD22 | | Ramos | |
| | | Max | | Max | | Max |
| | EC₅₀ | signal | EC₅₀ | signal | EC₅₀ | signal |
| Antibody | (M) | (gMFI) | (M) | (gMFI) | (M) | (gMFI) |
| REGN9220 | ND | 4.44E+02 | 8.50E−11^{†} | 4.58E+03 | 3.22E−10^{†} | 2.08E+04 |
| REGN9289 | ND | 4.14E+02 | 7.67E−11^{††} | 6.04E+03 | 8.76E−11^{††} | 2.26E+04 |
| REGN17596 | ND | 5.36E+02 | 3.71E−10^{†} | 5.77E+03 | 4.76E−10^{†} | 2.33E+04 |
| Comparator 1 | 2.28E−10 | 6.60E+03 | 1.91E−10 | 6.43E+03 | 1.90E−10 | 8.32E+03 |
| Control 1 | ND | 4.58E+02 | ND | 5.52E+02 | ND | 5.56E+02 |
| Control 2 | ND | 5.46E+02 | ND | 4.60E+02 | ND | 9.04E+02 |
| Isotype control | ND | 7.26E+02 | ND | 5.00E+02 | ND | 6.66E+02 |

ND: Not Determined because no dose dependent response was observed
Max signal (RLU) is the highest mean RLU value within tested dose-range.
^{†}While all data points are used to determine Maximum luciferase activity, for EC₅₀ calculation the values for the highest 2 antibody concentrations were removed due to a “hook effect”.
^{††}While all data points are used to determine Maximum luciferase activity, for EC₅₀ calculation the values for the highest 3 antibody concentrations were removed due to a “hook effect”.

In the presence of HEK293/hCD20 target cells lacking CD22, no response was seen with REGN9220, REGN9289, and REGN17596 nor with Isotype control. Only Comparator 1 led to a dose dependent increase in luciferase activity. In the presence of HEK293/hCD19/hCD20/hD22 target cells, REGN9220, REGN9289, and REGN17596 led to an increase in luciferase activity. Comparator 1 also led to a dose dependent increase in luciferase activity, whereas Isotype control did not. In the presence of the Ramos target cells, REGN9220, REGN9289, and REGN17596 led to an increase in luciferase activity. Comparator 1 also led to a dose dependent increase in luciferase activity, whereas Isotype control did not.

### Example 5: Potent Ability of Multispecific CD22×4-1BB Antibodies to Activate Primary T Cells

This example relates to an in vitro study demonstrating the ability of multispecific CD22×4-1BB antibodies to activate human primary T cells, as determined by release of the cytokines IL-2, IFN-γ, TNF-α, and GM-CSF, by engaging CD22 and 4-1BB. The ability of multispecific CD22×41BB antibodies to activate human primary T-cells by engaging CD22 and 4-1BB receptor to deliver “signal 2” was evaluated in the presence of a human embryonic kidney cancer cell line engineered to express CD20 and CD22 (HEK293/CD20/CD22) using a CD20×CD3 bispecific antibody to serve as “signal 1.” HEK293 cells expressing only CD20 were included as a control to measure activity that may occur in the absence of CD22 on antigen presenting cells. Additionally, a diffuse large B-cell lymphoma cell line that endogenously expresses hCD22, WSU-DLCL2, was included in testing multispecific CD22×41BB antibodies. As WSU-DLCL2 cells endogenously express CD20, a CD20×CD3 bispecific antibody was included to serve as “signal 1.” Lastly, conditions that included WSU-DLCL2 as target cells, in the absence of a CD3 bispecific were tested. Of note, unlike HEK293 cells, WSU cells provide detectable allogeneic stimulation of T-cells, where the TCR-MHC interaction serves as “signal 1’, in the absence CD3 antibody stimulation.

The following cell lines were used in this study: HEK293/CD20: HEK293 cells engineered to constitutively express full length human CD20 (amino acids M1-P297 of accession number NP_690605.1); HEK293/CD20/CD22 (high sort): HEK293/CD20 cells engineered to constitutively express full length human CD22 (amino acids M1-H847 of accession number NP_001762.2), then stained for CD22 expression and flow sorted to obtain a cell line with high human CD22 expression; and WSU-DLCL2: a human diffuse large B-cell lymphoma established from the pleural effusion of a 41-year-old Caucasian male with B-cell non-Hodgkin lymphoma in 1990. The following antibodies, as described in Example 1, were used in this study: REGN9220, REGN9289, REGN17596, Comparator 1, Control 1, Control 2, Isotype control, and the CD20×CD3 antibody odronextamab (U.S. Pat. No. 9,657,102).

Experimental Procedure: Human peripheral blood mononuclear cells (PBMCs) were isolated from a healthy donor leukocyte pack from Precision for Medicine using an EasySep™ Human T-Cell Isolation kit from StemCell Technologies, following the manufacturers recommendations. Subsequently, CD3+ T-cells were isolated from PBMCs using an EasySep™ Human CD3+ T Cell Isolation Kit from StemCell Technologies and following the manufacturer's recommended instructions.

Enriched CD3+ T-cells, resuspended in stimulation medium, were added into 96-well round bottom plates at 1×10⁵ cells/well. Target cells were added to CD3+ T-cells at 2.5×10⁴ cells/well for HEK293/CD20/CD22, HEK293/CD20 and WSU-DLCL2 cells. Following addition of cells, a constant of 0.2 nM odronextamab was added to wells containing HEK293/CD20/CD22 or HEK293/CD20 cells. A constant of 0.2 nM odronextamab or an isotype control was added to wells containing WSU-DLCL2 cells. Subsequently, the antibodies were titrated from 1.53 pM to 100 nM in a 1:4 dilution and added to wells. The final point of the 10-point dilution contained no titrated antibody. After plates were incubated for 72 hours at 37° C., 5% CO2, 4×5 μL supernatant was removed for measuring IL-2, IFN-γ, TNF-α, and GM-CSF. The amount of cytokine in assay supernatant was determined using AlphaLisa™ kits from PerkinElmer following the manufacturer's protocol. The cytokine measurements were acquired on a multilabel plate reader and values were reported as pg/mL. All serial dilutions were tested in triplicate.

The EC₅₀ values of the antibodies were determined from a four-parameter logistic equation over a 10-point dose-response curve using GraphPad Prism™ software. Signal recorded for the 10^{th} point on the dilution curve (no titrated antibody) was plotted at 381 fM. Maximal cytokine is given as the mean max response detected within the tested dose range.

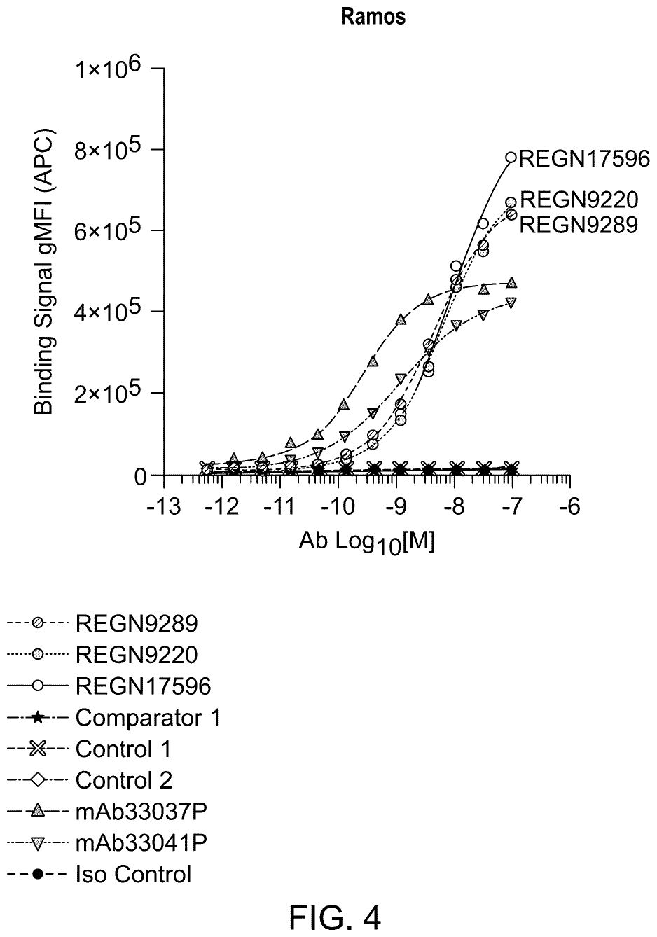

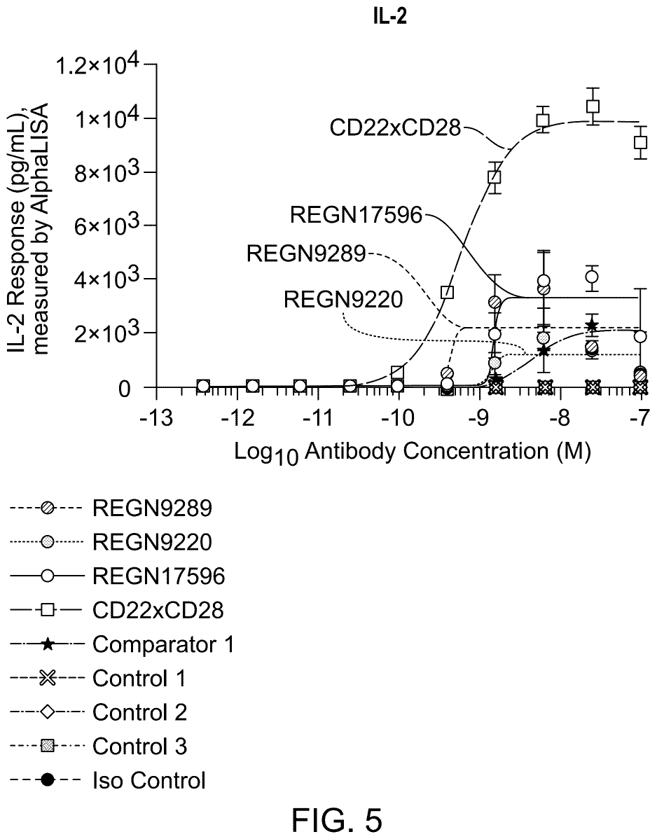

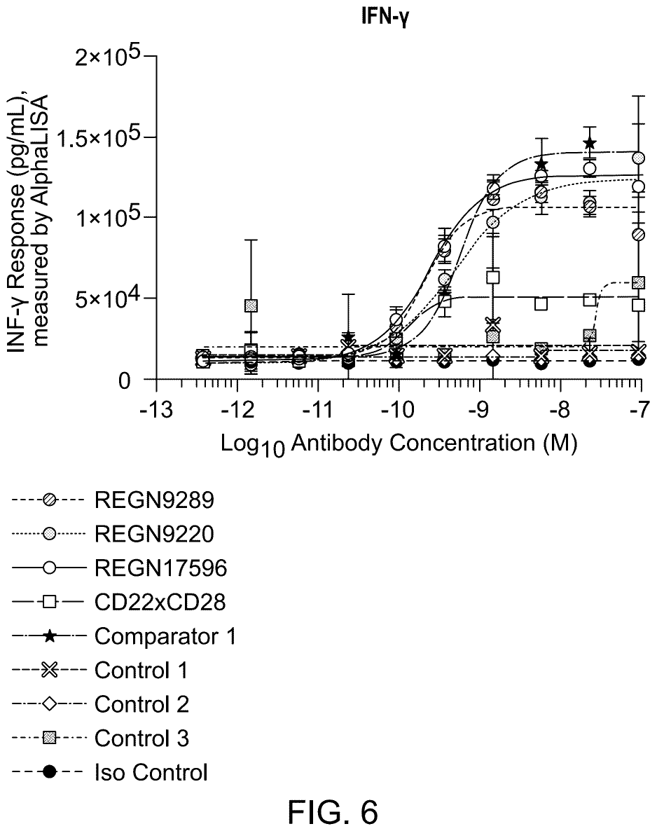

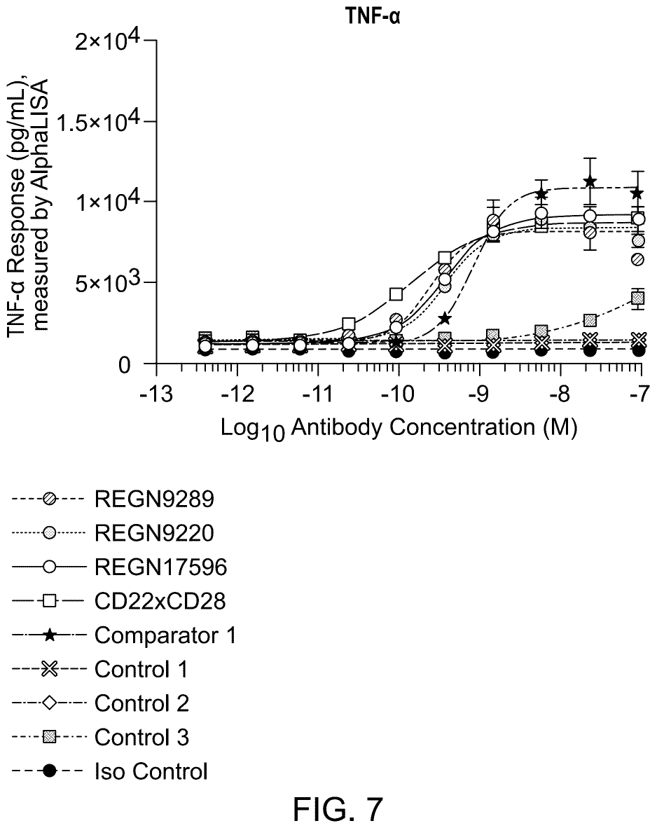

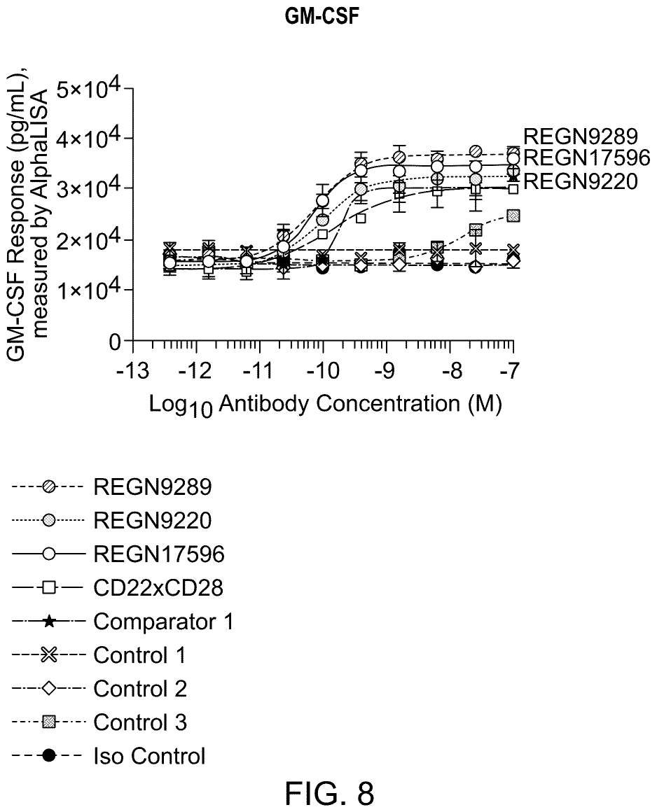

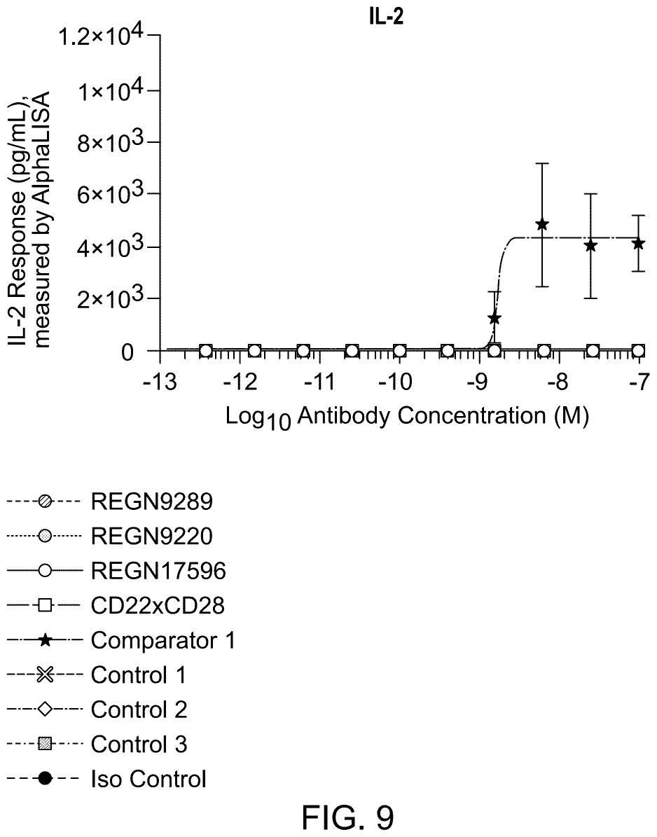

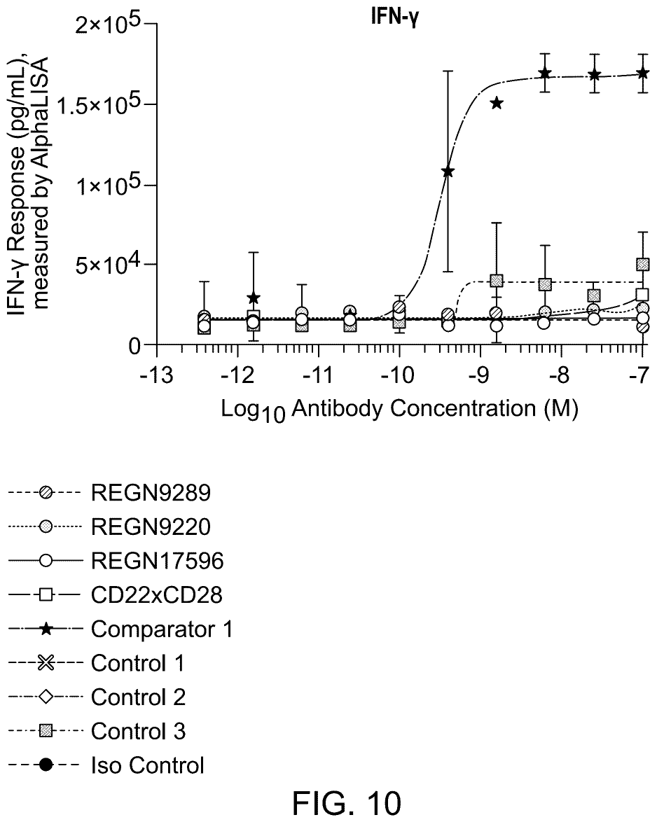

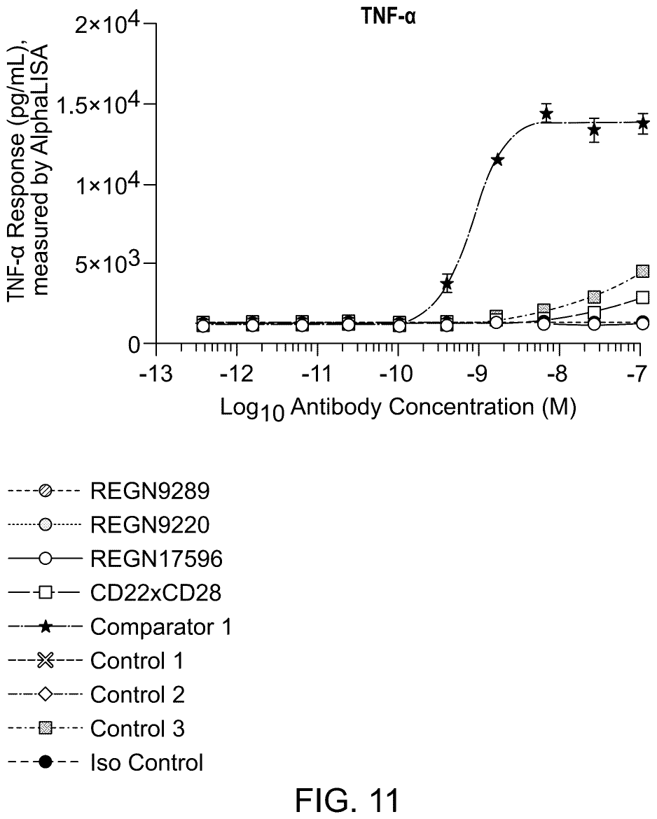

Results: REGN9220, REGN9289, REGN17596 induced higher IL-2, IFN-γ, TNF-α, and GM-CSF responses compared to Control 1, Control 2, and Isotype control in the presence of CD22-positive cells (HEK293/CD20/CD22), and “signal 1” provided by odronextamab (Tables 19-22; FIGS. 5-8), but not in the presence of CD22-negative cells (HEK293/CD20) (Tables 19-22; FIGS. 9-12). In contrast, Comparator 1 induced a dose dependent increase in cytokine release, irrespective of CD22 target expression (Tables 19-22; FIGS. 5-12). In the absence of ‘signal 1’ none of the antibodies lead to dose dependent enhancement of IL-2, IFN-γ, TNF-α, or GM-CSF release (Tables 19-22).

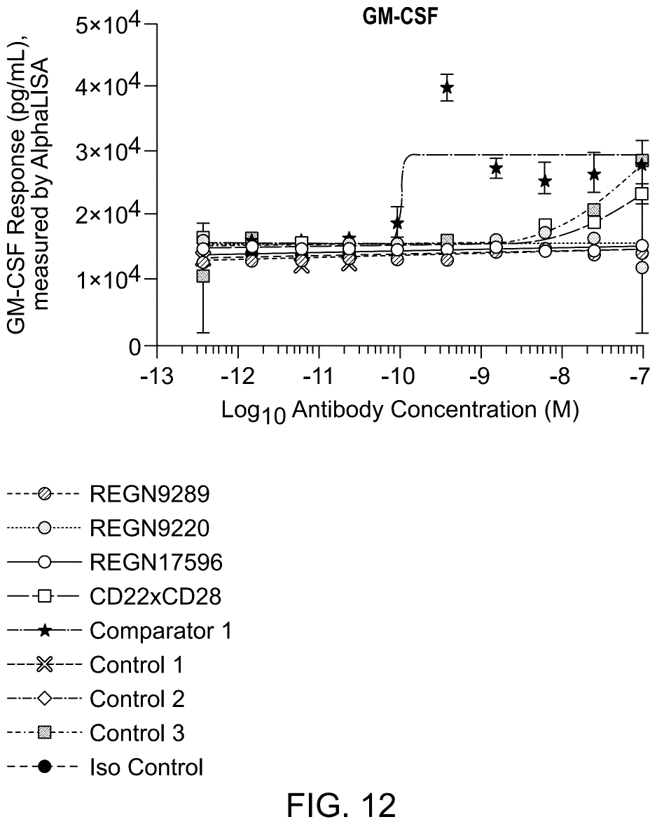

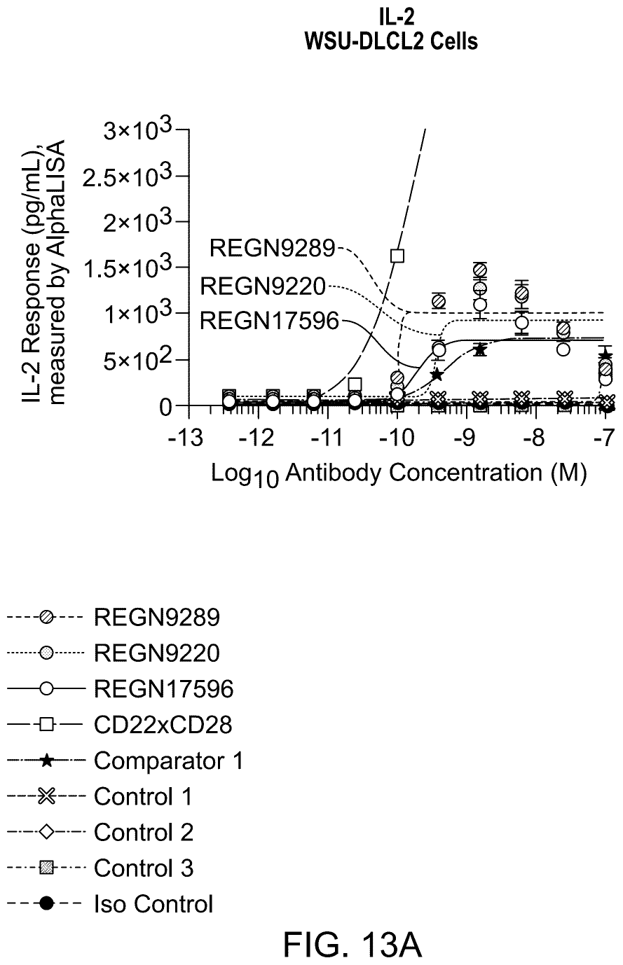

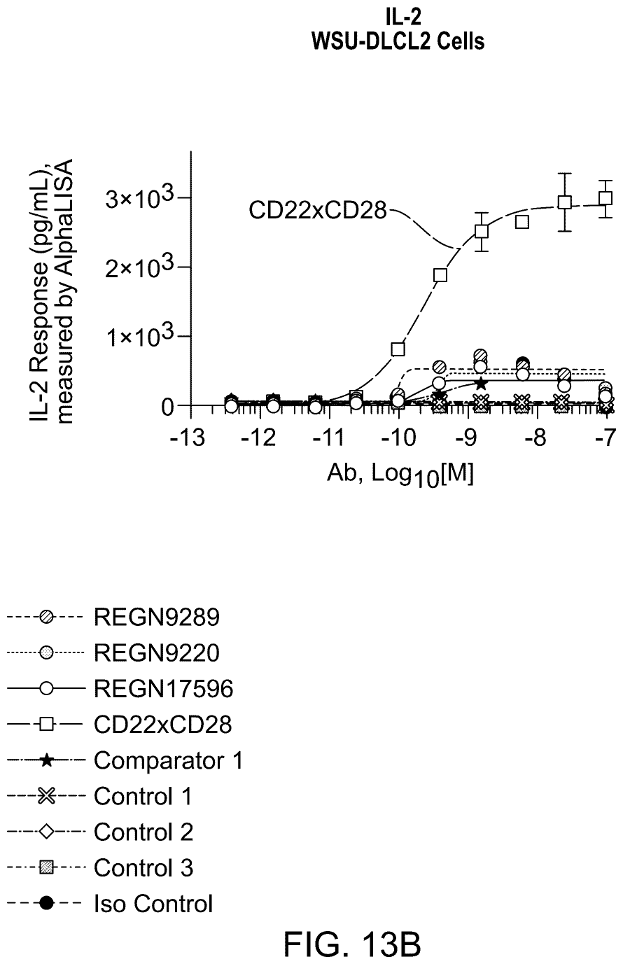

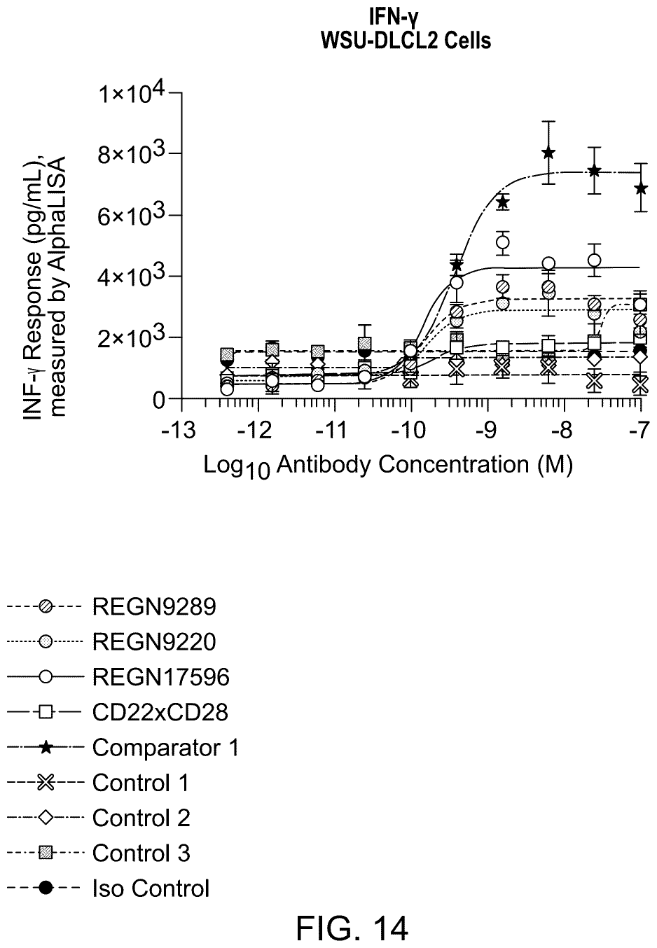

In the presence of allogeneic WSU-DLCL2 cells, and absence of CD3 bispecific antibody stimulation, REGN9220, REGN9289, and REGN17596, as well as Comparator 1, mediated a dose-dependent increase in IL-2 and GM-CSF (Tables 19 and 22). No dose dependent increase was observed for Control 1, Control 2, or Isotype control for either IL-2 or GM-CSF (Tables 19 and 22). No dose dependent response was observed for any antibody from IFN-γ or TNF-α (Tables 20 and 21). Addition of a fixed amount of 0.2 nM odronextamab in conditions with WSU-DLCL2 cells and primary human T-cells resulted in a dose dependent increase in IL-2, IFN-γ, TNF-α, and GM-CSF for REGN9220, REGN9289, and REGN17596 as well as for Comparator 1 (Tables 19-22; FIGS. 13-16). None of Control 1, Control 2, or Isotype control lead to dose dependent cytokine release (Tables 19-22; FIGS. 13-16).

Table 19 sets forth potency values and maximum IL-2 release for selected multispecific CD22×4-1BB antibodies.

TABLE 19
Potency Values and Maximum IL-2 Release for selected multispecific CD22x4-1BB Antibodies

| | HEK293/CD20/CD22 | | | | | | | |
|---|---|---|---|---|---|---|---|---|
| | high sort + | | HEK293/CD20 + | | WSU-DLCL2 + | | WSU-DLCL2 + | |
| | odronextamab | | odronextamab | | odronextamab | | Isotype control | |
| | EC₅₀ | MAX | EC₅₀ | MAX | EC₅₀ | MAX | EC₅₀ | MAX |
| Antibody | [M] | [pg/ml] | [M] | [pg/ml] | [M] | [pg/m]] | [M] | [pg/ml] |
| REGN17596 | 1.53E−09 | 4.07E+03 | ND | 4.02E−01 | 2.03E−10 | 1.10E+03 | 1.051E−9 | 1.36E+02 |
| REGN9289 | 4.40E−10 | 3.69E+03 | ND | 5.50E−01 | 1.04E−10 | 1.46E+03 | 2.50E−11 | 1.10E+02 |
| REGN9220 | 1.46E−09 | 1.82E+03 | ND | 1.50E−02 | 3.64E−10 | 1.27E+03 | 1.19E−10 | 1.52E+02 |
| Comparator 1 | 4.70E−09 | 2.28E+03 | 1.70e−009 | 4.82E+03 | 5.02E−10 | 9.00E+02 | 1.11E−10 | 1.55E+02 |
| Control 1 | ND | 3.96E+01 | ND | 9.00E−03 | ND | 8.09E+01 | NC | 6.70E+01 |
| Control 2 | ND | 1.06E+00 | ND | 1.38E−01 | ND | 7.61E+01 | ND | 6.88E+01 |
| Isotype control | ND | 1.31E+00 | ND | 5.60E−02 | ND | 2.86E+01 | ND | 6.45E+01 |

ND: Not Determined because no dose dependent response was observed
NC: Not calculated because the data did not fit a 4-parameter logistic equation

TABLE 20
Potency Values and Maximum IFN-γ Release for selected multispecific CD22x4-1BB Antibodies

| | HEK293/CD20/CD22 | | | | | | | |
|---|---|---|---|---|---|---|---|---|
| | high sort + | | HEK293/CD20 + | | WSU-DLCL2 + | | WSU-DLCL2 + | |
| | odronextamab | | odronextamab | | odronextamab | | Isotype control | |
| | EC₅₀ | MAX | EC₅₀ | MAX | EC₅₀ | MAX | EC₅₀ | MAX |
| Antibody | [M] | [pg/ml] | [M] | [pg/ml] | [M] | [pg/ml] | [M] | [pg/ml] |
| REGN17596 | 2.61E−10 | 1.31E+05 | ND | 1.86E+04 | 1.44E−10 | 5.08E+03 | ND | 0 |
| REGN9289 | 2.15E−10 | 1.16E+05 | ND | 2.02E+04 | 1.51E−10 | 3.68E+03 | ND | 0 |
| REGN9220 | 5.07E−10 | 1.36E+05 | ND | 2.13E+04 | 1.68E−10 | 3.46E+03 | ND | 0 |
| Comparator 1 | 6.15E−10 | 1.47E+05 | 3.34E−10 | 1.70E+05 | 3.70E−10 | 8.06E+03 | ND | 0 |
| Control 1 | ND | 3.35E+04 | NC | 3.52E+04 | ND | 1.04E+03 | ND | 0 |
| Control 2 | ND | 1.86E+04 | ND | 2.30E+04 | ND | 1.49E+03 | ND | 0 |
| Isotype control | ND | 1.76E+04 | ND | 1.68E+04 | ND | 1.67E+03 | ND | 0 |

ND: Not Determined because no dose dependent response was observed
NC: Not calculated because the data did not fit a 4-parameter logistic equation

Table 21 sets forth potency values and maximum TNF-α release for selected antibodies.

TABLE 21
Potency Values and Maximum TNF-α Release for selected Antibodies

| | HEK293/CD20/CD22 | | | | | | | |
|---|---|---|---|---|---|---|---|---|
| | high sort + | | HEK293/CD20 + | | WSU-DLCL2 + | | WSU-DLCL2 + | |
| | odronextamab | | odronextamab | | odronextamab | | Isotype control | |
| | EC₅₀ | MAX | EC₅₀ | MAX | EC₅₀ | MAX | EC₅₀ | MAX |
| Antibody | [M] | [pg/ml] | [M] | [pg/ml] | [M] | [pg/ml] | [M] | [pg/ml] |
| REGN17596 | 3.85E−10 | 9.48E+03 | ND | 1.49E+03 | 1.32E−10 | 4.13E+03 | ND | 1.15E+02 |
| REGN9289 | 2.58E−10 | 9.32E+03 | ND | 1.34E+03 | 8.98E−11 | 3.19E+03 | ND | 7.47E+01 |
| REGN9220 | 3.95E−10 | 9.05E+03 | ND | 1.42E+03 | 1.48E−10 | 3.22E+03 | ND | 9.74E+01 |
| Comparator 1 | 8.84E−10 | 1.14E+04 | 7.69E−10 | 1.43E+04 | 3.48E−10 | 3.28E+03 | ND | 1.05E+02 |
| Control 1 | ND | 1.41E+03 | ND | 1.37E+03 | ND | 1.92E+03 | ND | 9.88E+01 |
| Control 2 | ND | 1.50E+03 | ND | 1.33E+03 | ND | 2.07E+03 | ND | 9.82E+01 |
| Isotype control | ND | 9.47E+02 | ND | 1.24E+03 | ND | 1.81E+03 | ND | 7.35E+01 |

ND: Not Determined because no dose dependent response was observed
NC: Not calculated because the data did not fit a 4-parameter logistic equation

Table 22 sets forth potency values and maximum GM-CSF release for selected multispecific CD22×4-1BB antibodies.

TABLE 22
Potency Values and Maximum GM-CSF Release for selected Antibodies

| | HEK293/CD20/CD22 | | | | | | | |
|---|---|---|---|---|---|---|---|---|
| | high sort + | | HEK293/CD20 + | | WSU-DLCL2 + | | WSU-DLCL2 + | |
| | odronextamab | | odronextamab | | odronextamab | | Isotype control | |
| | EC₅₀ | MAX | EC₅₀ | MAX | EC₅₀ | MAX | EC₅₀ | MAX |
| Antibody | [M] | [pg/ml] | [M] | [pg/ml] | [M] | [pg/ml] | [M] | [pg/ml] |
| REGN17596 | 6.98E−11 | 3.60E+04 | ND | 1.52E+04 | 9.36E−11 | 2.72E+04 | 7.87E−11 | 1.09E+03 |
| REGN9289 | 7.57E−11 | 3.75E+04 | ND | 1.47E+04 | 8.75E−11 | 2.08E+04 | 2.43E−11 | 1.20E+03 |
| REGN9220 | 9.45E−11 | 3.38E+04 | ND | 1.73E+04 | 1.17E−10 | 2.09E+04 | 1.04E−11 | 1.27E+03 |
| Comparator 1 | 1.78E−10 | 3.24E+04 | 1.04E−10 | 3.98E+04 | 2.47E−10 | 2.40E+04 | 9.83E−11 | 1.36E+03 |
| Control 1 | ND | 1.66E+04 | ND | 1.46E+04 | NC | 1.12E+04 | NC | 6.86E+02 |
| Control 2 | ND | 1.89E+04 | ND | 1.53E+04 | ND | 1.18E+04 | ND | 5.98E+02 |
| Isotype control | ND | 1.66E+04 | ND | 1.57E+04 | ND | 1.18E+04 | ND | 4.96E+02 |

ND: Not Determined because no dose dependent response was observed
NC: Not calculated because the data did not fit a 4-parameter logistic equation

### Example 6: Potent Ability of Multispecific CD22×4-1BB Antibodies to Enhance CD20×CD3-Directed Killing of Ramos Cells

This example relates to an in vitro study performed to demonstrate the ability of multispecific CD22×4-1BB antibodies to enhance CD20×CD3-directed killing by hPBMC of cells of the CD22+ B cell lymphocyte cell line, Ramos.

Enhancement of CD20×CD3 mediated killing by the multispecific CD22×4-1BB antibody REGN9220 was evaluated in a 7-10 day cytotoxicity assay targeting Ramos cells. PBMC previously isolated from normal human donor leukopacs were thawed, washed, resuspended in tissue culture medium (R10) and plated in 96 well flat bottom plates at a 1:1 ratio with Ramos cells engineered to stably express cytoplasmic GFP (Ramos/GFP). A fixed concentration (200 pM) of the CD20×CD3 bispecific antibody, odronextamab (U.S. Pat. No. 9,657,102) diluted in R10 was added to the wells with or without 9-fold serial dilutions of REGN9220 or the CD22×CD28 bispecific antibody REGN5837 [U.S. Pat. No. 11,396,544] as a positive control (concentration range: 600 nM to 91.4 pM). The plates were incubated for up to 240 hours at 37° C./5% CO2, with scanning for green-fluorescence using a fluorescent microscope Incucyte XS5 platform every 4 hours. Ramos/GFP cell presence was quantified as the green-fluorescence area (GFA) detected at each timepoint determined by scanning of plates. Cytotoxicity was based on the cell surface area occupied by Ramos/GFP normalized to the surface area occupied by Ramos/GPF in the presence of PBMC only. Percent cytotoxicity of Ramos/GFP cells at any timepoint along the course of the acquisition period was defined as:

$$Percent cytotoxocity = 100 \times [ 1 - ( G F A within tested dose range / Mean G F A of P B M C + Ramos cells without antibody ) ]$$

Max cytotoxicity was the highest cytoxicity across the range of CD22×41BB concentrations tested in the assay. Fold increase in % cytotoxicity was calculated by dividing the max cytotoxicity of 200 pM odronextamab+REGN9220 by the cytotoxicity of 200 pM odronextamab alone. Each condition was performed in triplicate and the experiment was repeated twice with the same donor PBMC.
The EC₅₀ values of the Ramos/GFP survival were determined using GraphPad Prism™ software from a four-parameter logistic equation over a 6-point dose-response curve, where the 6^{th} dilution point contained no REGN9220 or REGN5837 (200 pM odronextamab only).

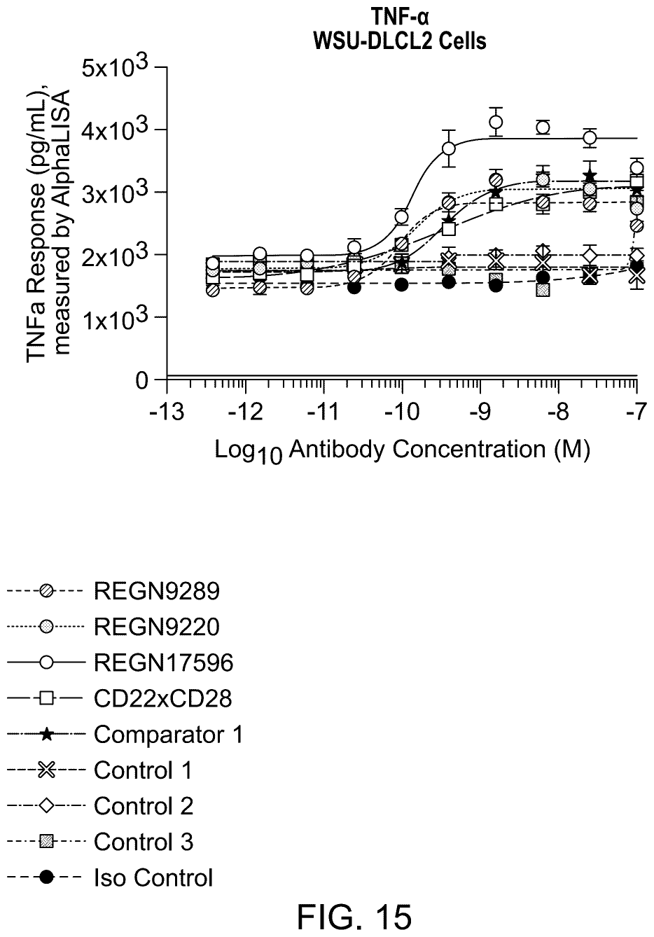

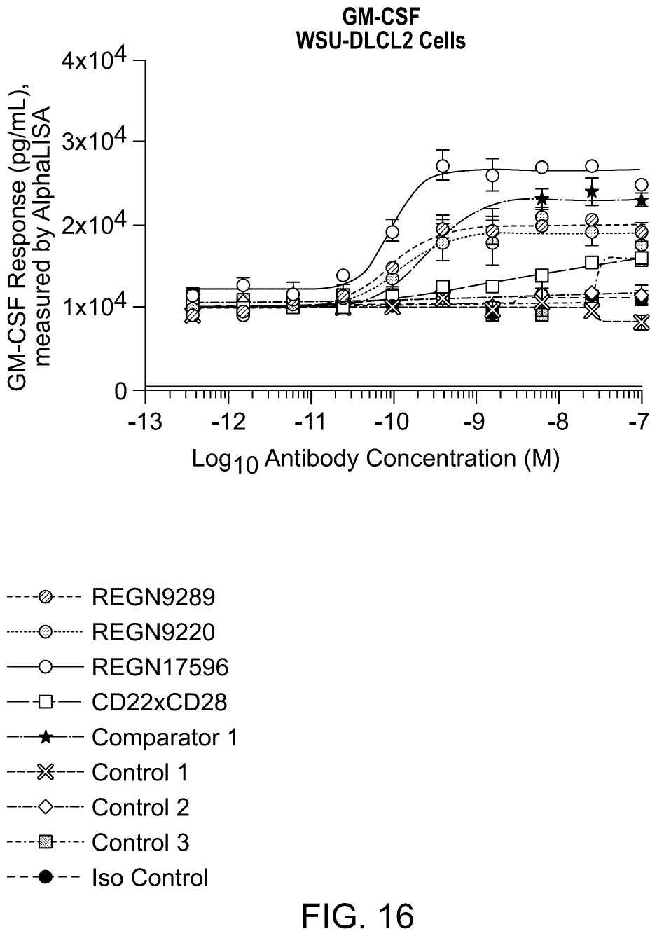

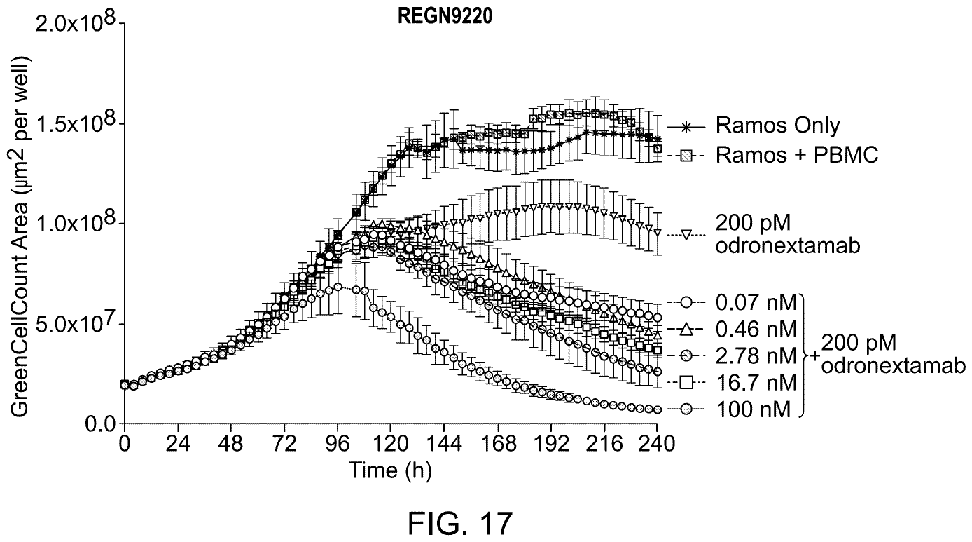

Results: The addition of a dose titration of REGN9220 to a constant amount of odronextamab resulted in a dose dependent increase of T-cell targeted cytoxicity of Ramos/GFP (definied by a decrease in the green-fluorescent occupied area) over cytocixity in the presence of 200 pM CD20×CD3 alone (FIG. 17). This was similar to the enhancement of cytoxicity seen for REGN5837 (FIG. 18). A 4 to 7.7 fold increase in maximum cytotoxicity, single digit nanomolar potency, was observed for REGN9220 (Table 23). A dose dependent decrease in survival was observed for REGN9220 by 192 hours (FIG. 19).

Table 23 sets forth the Ramos/GFP targeted cytotoxicity mediated by 200 pM odronextamab+/−a titration of REGN9220 at 168 hours and PBMC (1:1 E:T).

TABLE 23
Summary of Cytotoxicity Measures

| | Experiment 1 | Experiment 2 |
|---|---|---|
| Cytotoxicity (%) of 200 pM | 10.90% | 8.97% |
| CD20 × CD3 only | | |
| Max Cytotoxicity (%)CD20 × CD3 + | 83.50% | 36.20% |
| CD22 × 41BB | | |
| Fold increase in % cytotoxicy | 7.7 | 4.0 |
| CD22 × 41BB EC50 (M) | NC | 6.75E−09 |

NC: Not calculated because the data did not fit a 4-parameter logistic equation

### Example 7: Robust Anti-Tumor Efficacy of Multispecific CD22×4-1BB Antibody REGN9220 in the Syngenic MC38/hCD20/hCD22 Tumor Model

To evaluate the anti-tumor efficacy of CD22×4-1BB bispecific antibody (bsAb) in combination with odronextamab in animals with a fully intact immune system, mice were genetically engineered to express humanized CD20, CD22, CD3, CD28, and 4-1BB. The anti-tumor activity of CD22×4-1BB bsAb REGN9220 was then characterized in an MC38 (colon carcinoma) tumor model where the cell line had been modified to ectopically express human CD20 and human CD22 (MC38hCD20hCD22). On day 0, animals humanized for CD20, CD22, CD3, CD28, and 4-1BB on the C57/Bl6 background were subcutaneously implanted with 5×10⁵ MC38hCD20hCD22 tumor cells. Mice were randomized to receive blinded treatments of either non-targeted isotype controls or test articles (REGN9220 [CD22×4-1BB)], odronextamab [CD20×CD3]) which were administered as monotherapy or in combination by intraperitoneal injection at specified concentrations on day 7, 10, 14, 17, and 21 days post implantation. These studies demonstrate that while monotherapy treatment with R9220 (5 mg/kg) or odronextamab (2.5 mg/kg) induced modest tumor growth inhibition with enhanced survival, the combination of R9220 with odronextamb induced potent dose-dependent anti-tumor activity with a 75% tumor rejection rate when odronextamab was dosed with R9220 at 5 mg/kg, a 12.5% rejection rate when odronextamab was dosed with R9220 at 1 mg/kg, and a loss of combinatorial activity when R9220 was dosed at 0.2 mg/kg.

### Experimental Procedure

On day 0, C57/BL6 animals humanized for CD20, CD22, CD3, CD28, and 4-1BB were subcutaneously implanted with 5×10⁵ MC38hCD20hCD22 tumor cells (MC38 colon carcinoma cell line (NIH) was lentivirally-transduced to express human CD20 and human CD22). Mice were randomized to receive blinded treatments of either non-targeted isotype controls or test articles (CD22×4-1BB, odronextamab [CD20×CD3]) which were administered as monotherapy or in combination by intraperitoneal injection at specified concentrations on day 7, 10, 14, 17, and 21 days post implantation. Tumor growth was monitored over time using digital caliper (VWR) measurements of X and Y diameter (perpendicular measurements of length and width). Tumor volume was calculated (X*Y*(X/2) where X is the shorter diameter). Mice were euthanized when the tumor reached a designated tumor endpoint (tumor diameter >20 mm, or tumor ulceration).

### Results, Summary and Conclusion

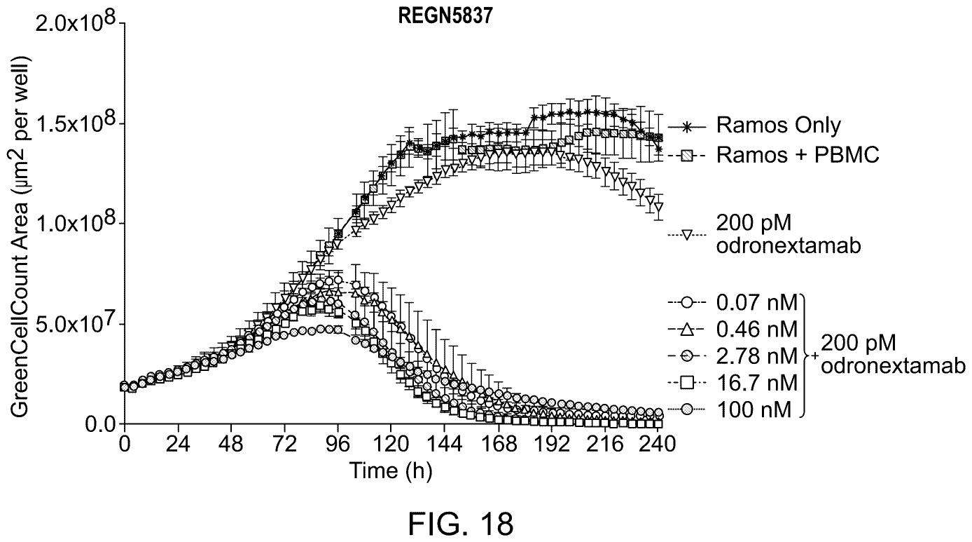

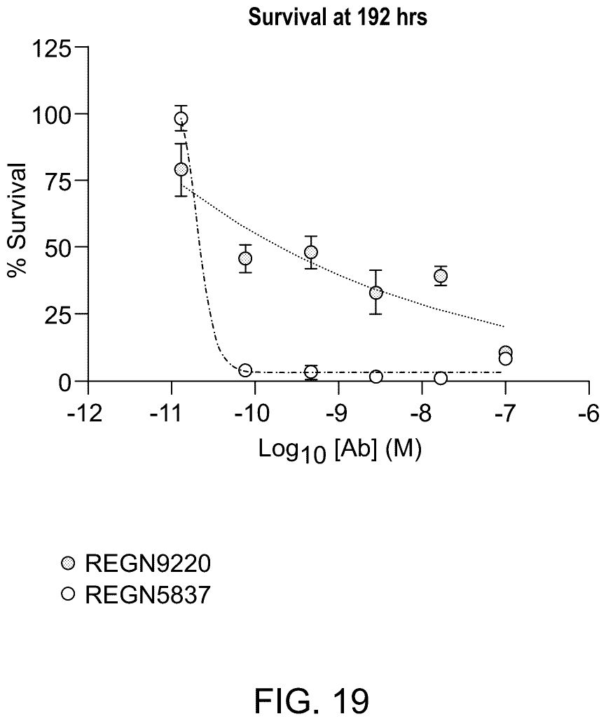

While Odronextamab monotherapy (2.5 mg/kg) initially suppressed MC38hCD20hCD22 tumor growth (FIG. 20), resulting in an enhancement of survival in comparison to isotype treated control animals (FIG. 21), all animals eventually succumbed to tumor burden. Monotherapy treatment with CD22×4-1BB (5 mg/kg) also modestly inhibited tumor growth (FIG. 20) and significantly enhanced survival (FIG. 21), but treatment did not result in any curative responses. However, combination treatment of CD22×4-1BB with odronextamb induced potent dose-dependent anti-tumor activity with a 75% tumor rejection rate when CD22×4-1BB was dosed at 5 mg/kg, a 12.5% rejection rate at 1 mg/kg and a loss of combinatorial activity with CD22×4-1BB was dosed at 0.2 mg/kg. Therefore, this data demonstrate that the synergistic combination of CD22×4-1BB and odronextamab can induce dose-dependent curative anti-tumor activity.

### Example 8: Robust Anti-Tumor Efficacy of Multispecific CD22×4-1BB Antibody REGN9289 in the Syngenic MC38/hCD20/hCD22 Tumor Model

To evaluate the anti-tumor efficacy of CD22×4-1BB bispecific antibody (bsAb) in combination with odronextamab in animals with a fully intact immune system, mice were genetically engineered to express humanized CD20, CD22, CD3, CD28, and 4-1BB. The anti-tumor activity of CD22×4-1BB bsAb was then characterized in an MC38 (colon carcinoma) tumor model where the cell line had been modified to ectopically express human CD20 and human CD22 (MC38hCD20hCD22). On day 0, animals humanized for CD20, CD22, CD3, CD28, and 4-1BB on the C57/Bl6 background were subcutaneously implanted with 5×10⁵ MC38hCD20hCD22 tumor cells. Mice were randomized to receive blinded treatments of either non-targeted isotype controls or test articles (REGN9289 [CD22×4-1BB], odronextamab [CD20×CD3]) which were administered as monotherapy or in combination by intraperitoneal injection at specified concentrations on day 7, 10, 14, 17, and 21 days post implantation. These studies demonstrate that while odronextamab (2.5 mg/kg) monotherapy induced modest tumor growth inhibition with enhanced survival, the addition of CD22×4-1BB bsAb to odronextamb induced potent dose-dependent anti-tumor activity with a 75% tumor rejection rate when odronextamab was dosed with REGN9289 at 5 mg/kg and a 13% rejection rate when odronextamab was dosed with REGN9289 at 1 mg/kg.

### Experimental Procedure

On day 0, C57/BL6 animals humanized for CD20, CD22, CD3, CD28, and 4-1BB were subcutaneously implanted with 5×10⁵ MC38hCD20hCD22 tumor cells (MC38 colon carcinoma cell line (NIH) was lentivirally-transduced to express human CD20 and human CD22). Mice were randomized to receive blinded treatments of either non-targeted isotype controls (EGFRV3×CD3 or EGFR×4-1BB) or test articles (REGN9289 [CD22×4-1BB], odronextamab [CD20×CD3]) which were administered as monotherapy or in combination by intraperitoneal injection at specified concentrations on day 7, 10, 14, 17, and 21 days post implantation. Tumor growth was monitored over time using digital caliper (VWR) measurements of X and Y diameter (perpendicular measurements of length and width). Tumor volume was calculated (X*Y*(X/2) where X is the shorter diameter). Mice were euthanized when the tumor reached a designated tumor endpoint (tumor diameter >20 mm, or tumor ulceration).

At 4 hours post the first antibody treatment on day 7, blood was collected from submandibular vein into microtainer serum tubes (BD 365967). Cytokine concentrations were analyzed using V-plex Human ProInflammatory-10 Plex kit following the manufacturer's instructions (Meso Scale Diagnostics).

### Results, Summary and Conclusion

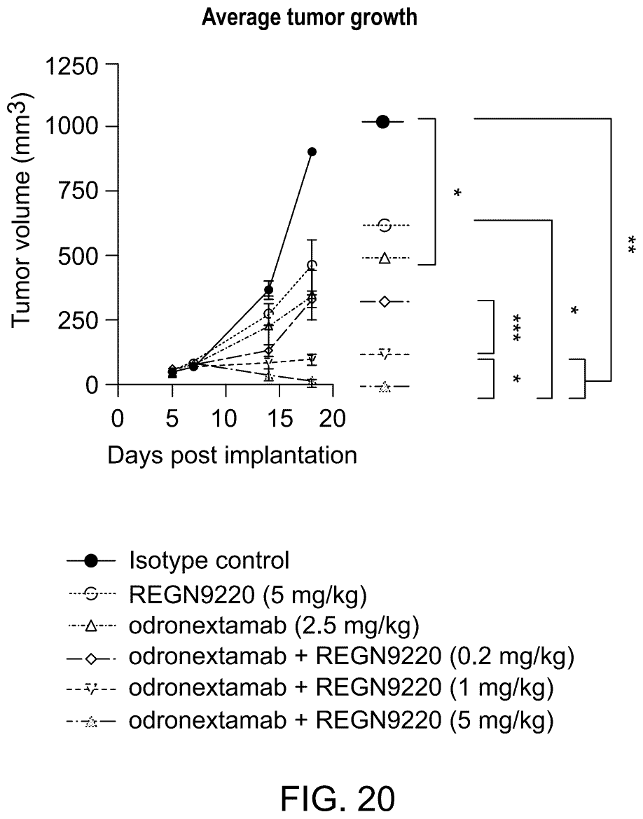

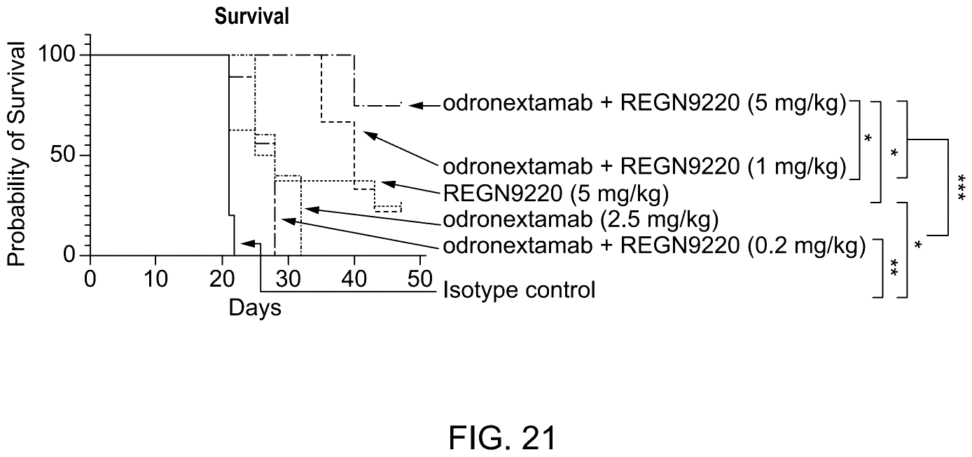

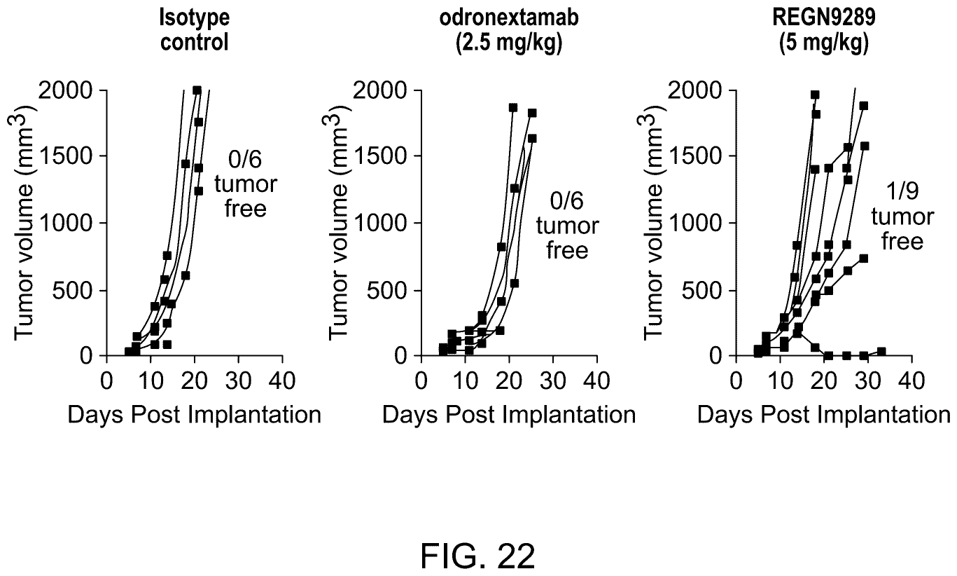

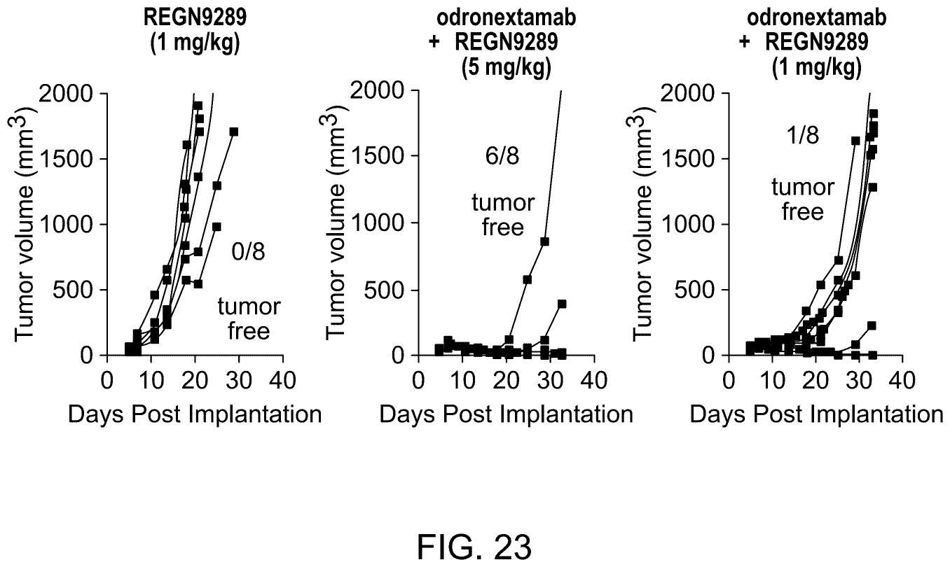

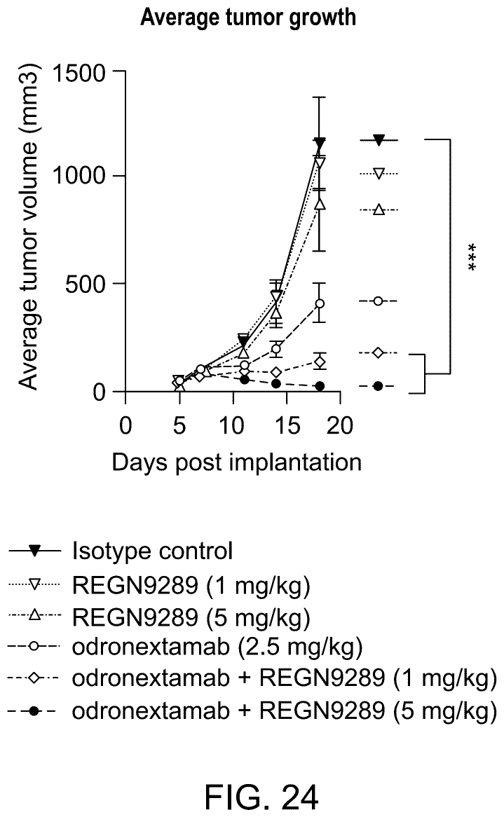

While Odronextamab monotherapy (2.5 mg/kg) initially suppressed MC38hCD20hCD22 tumor growth, resulting in an enhancement of survival in comparison to isotype treated control animals, all animals eventually succumbed to tumor burden (FIGS. 22-25). However, the addition of REGN9289 to odronextamb potently induced dose-dependent anti-tumor activity with a 75% tumor rejection rate when CD22×4-1BB was dosed at 5 mg/kg and a 13% rejection rate when CD22×4-1BB was dosed at 1 mg/kg (FIGS. 22-24). And while R9289 enhances odronextamab anti-tumor immunity, the addition of CD22×41BB antibody does not augment odronextamab-induced serum cytokine production 4 hrs post dose 1 (FIG. 26). Therefore, these data demonstrate that the synergistic combination of CD22×4-1BB and odronextamab can induce dose-dependent curative anti-tumor activity without enhancing serum cytokine production in tumor bearing animals.

### Example 9: Robust Anti-Tumor Efficacy of Multispecific CD22×4-1BB Antibody REGN17596 in the Syngenic MC38/hCD20/hCD22 Tumor Model

To evaluate the anti-tumor efficacy of CD22×4-1BB bispecific antibody (bsAb) in combination with odronextamab in animals with a fully intact immune system, mice were genetically engineered to express humanized CD20, CD22, CD3, CD28, and 4-1BB. The anti-tumor activity of CD22×4-1BB bsAb was then characterized in an MC38 (colon carcinoma) tumor model where the cell line had been modified to ectopically express human CD20 and human CD22 (MC38hCD20hCD22). On day 0, animals humanized for CD20, CD22, CD3, CD28, and 4-1BB on the C57/Bl6 background were subcutaneously implanted with 5×10⁵ MC38hCD20hCD22 tumor cells. Mice were randomized to receive blinded treatments of either non-targeted isotype controls or test articles (REGN17596 [CD22×4-1BB], odronextamab [CD20×CD3]) which were administered as monotherapy or in combination by intraperitoneal injection at specified concentrations on day 7, 10, 14, 17, and 21 days post implantation. These studies demonstrate that while monotherapy treatment with REGN17596 (5 mg/kg) or odronextamab (2.5 mg/kg) induced modest tumor growth inhibition with enhanced survival, the combination of CD22×4-1BB bsAb with odronextamb induced potent dose-dependent anti-tumor activity with a 67% tumor rejection rate when odronextamab was dosed with REGN17596 at 5 mg/kg, a 22% rejection rate when odronextamab was dosed with REGN17596 at 1 mg/kg, and an 11% rejection rate when REGN17596 was dosed at 0.2 mg/kg.

### Experimental Procedure

On day 0, C57/BL6 animals humanized for CD20, CD22, CD3, CD28, and 4-1BB were subcutaneously implanted with 5×10⁵ MC38hCD20hCD22 tumor cells (MC38 colon carcinoma cell line (NIH) was lentivirally-transduced to express human CD20 and human CD22). Mice were randomized to receive blinded treatments of either non-targeted isotype controls or test articles (REGN17596 [CD22×4-1BB], odronextamab [CD20×CD3]) which were administered as monotherapy or in combination by intraperitoneal injection at specified concentrations on day 7, 10, 14, 17, and 21 days post implantation. Tumor growth was monitored over time using digital caliper (VWR) measurements of X and Y diameter (perpendicular measurements of length and width). Tumor volume was calculated (X*Y*(X/2) where X is the shorter diameter). Mice were euthanized when the tumor reached a designated tumor endpoint (tumor diameter >20 mm, or tumor ulceration.

At 4 hours post the first antibody treatment on day 7, blood was collected from submandibular vein into microtainer serum tubes (BD 365967). Cytokine concentrations were analyzed using V-plex Human ProInflammatory-10 Plex kit following the manufacturer's instructions (Meso Scale Diagnostics).

### Results Summary and Conclusion

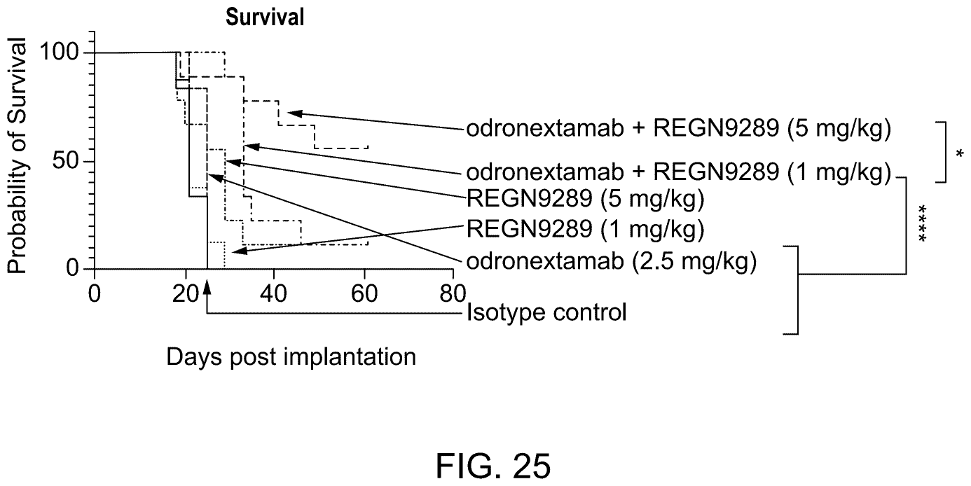

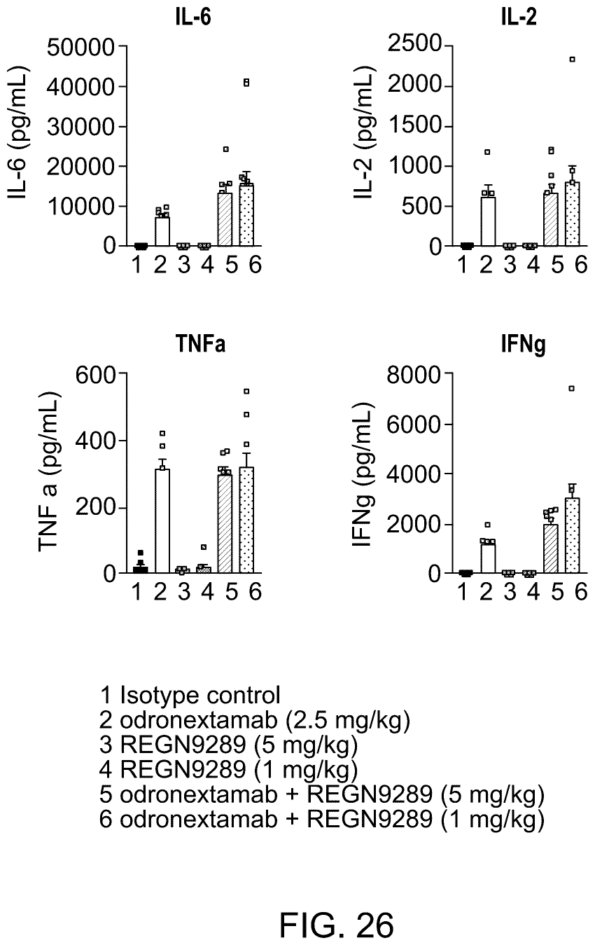

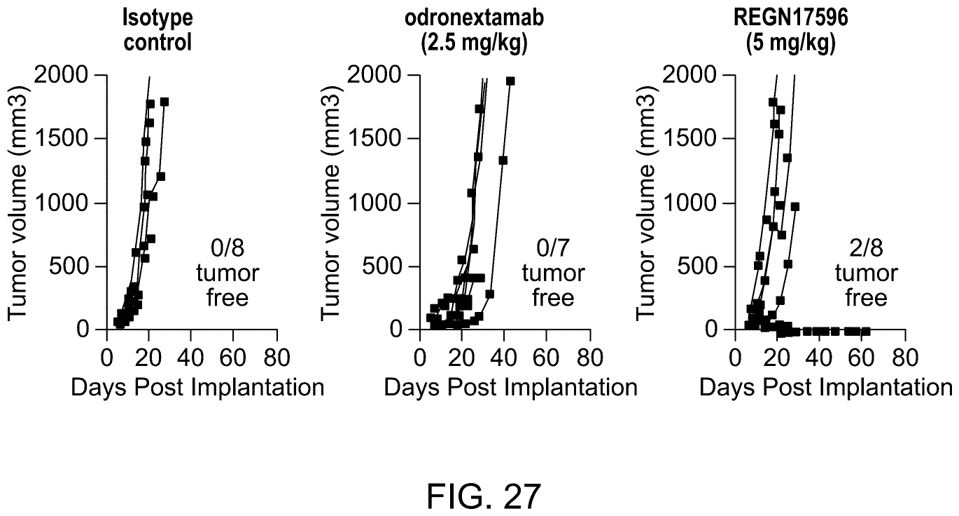

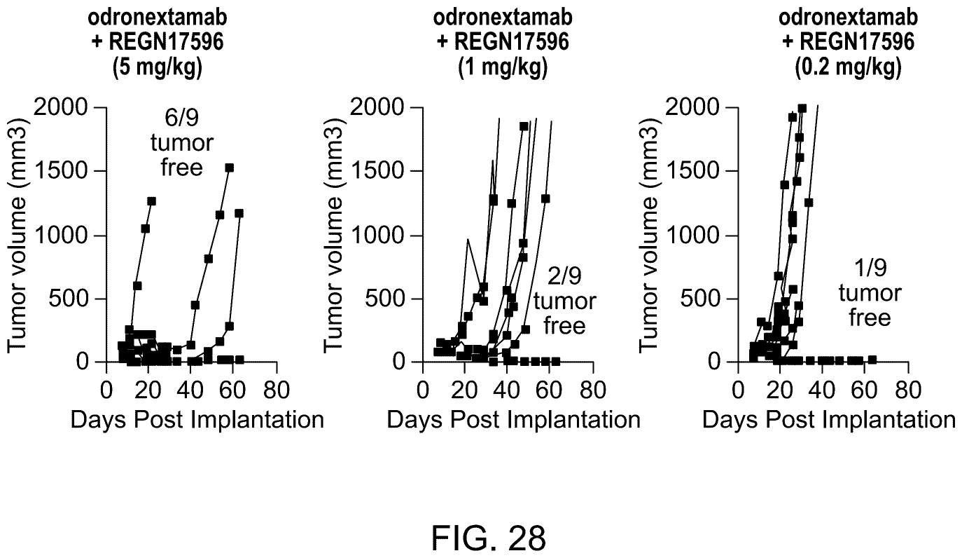

While Odronextamab monotherapy (2.5 mg/kg) initially suppressed MC38hCD20hCD22 tumor growth, resulting in an enhancement of survival in comparison to isotype treated control animals, all animals eventually succumbed to tumor burden (FIGS. 27-29). Monotherapy treatment with REGN17596 (5 mg/kg) induced modest tumor rejection with 25% of the animals tumor-free at the end of the experiment (FIGS. 27-29). However, combination treatment of REGN17596 with odronextamb induced potent dose-dependent anti-tumor activity with a 66% tumor rejection rate when CD22×4-1BB was dosed at 5 mg/kg, an 22% rejection rate at 1 mg/kg and a 11% rejection rate when CD22×4-1BB was dosed at 0.2 mg/kg (FIGS. 27-29). And while R17596 enhances odronextamab efficacy, the addition of CD22×41BB antibody does not augment odronextamab-induced serum cytokine production 4 hrs post dose 1 (FIG. 30). Therefore, these data demonstrate that the synergistic combination of CD22×4-1BB and odronextamab can induce dose-dependent curative anti-tumor activity without enhancing serum cytokine production in tumor bearing animals.

### Example 10: Immunophenotyping of Intratumoral and Peripheral T Cell Responses Following Treatment with CD22×4-1BB Antibodies in Combination with Odronextamab

To investigate how the addition of CD22×41BB to odronextamab augments anti-tumor immunity, intratumoral and peripheral T cell responses were immunopheotyped 14 days post tumor implantantion.

### Experimental Procedure

On day 0, C57/BL6 animals humanized for CD20, CD22, CD3, CD28, and 4-1BB were subcutaneously implanted with 5×10⁵ MC38hCD20hCD22 tumor cells (MC38 colon carcinoma cell line (NIH) was lentivirally-transduced to express human CD20 and human CD22). Mice were randomized to receive blinded treatments of either non-targeted isotype controls or test articles (REGN9289 [CD22×4-1BB], odronextamab [CD20×CD3]) which were administered as monotherapy or in combination by intraperitoneal injection at specified concentrations on day 7 and 10 post implantation. Tumor growth was monitored over time using digital caliper (VWR) measurements of X and Y diameter (perpendicular measurements of length and width). Tumor volume was calculated (X*Y*(X/2) where X is the shorter diameter). Mice were euthanized on day 14 to characterize intratumoral and peripheral T cell compartments.

### Results Summary and Conclusion

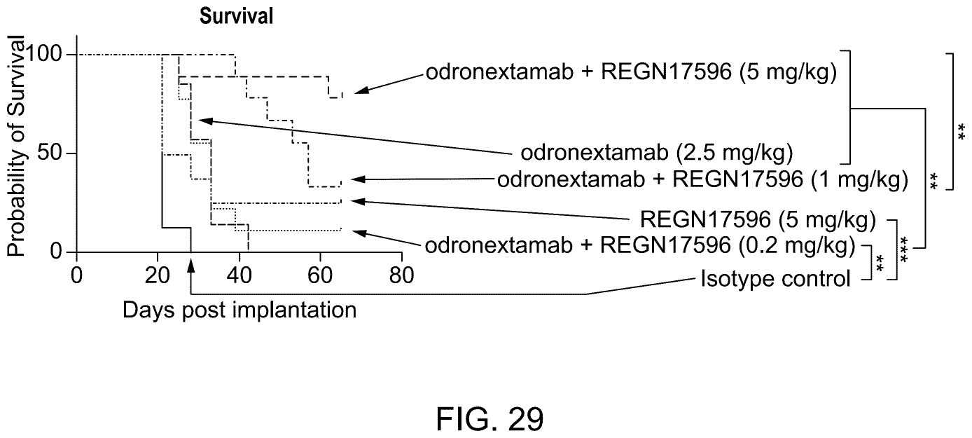

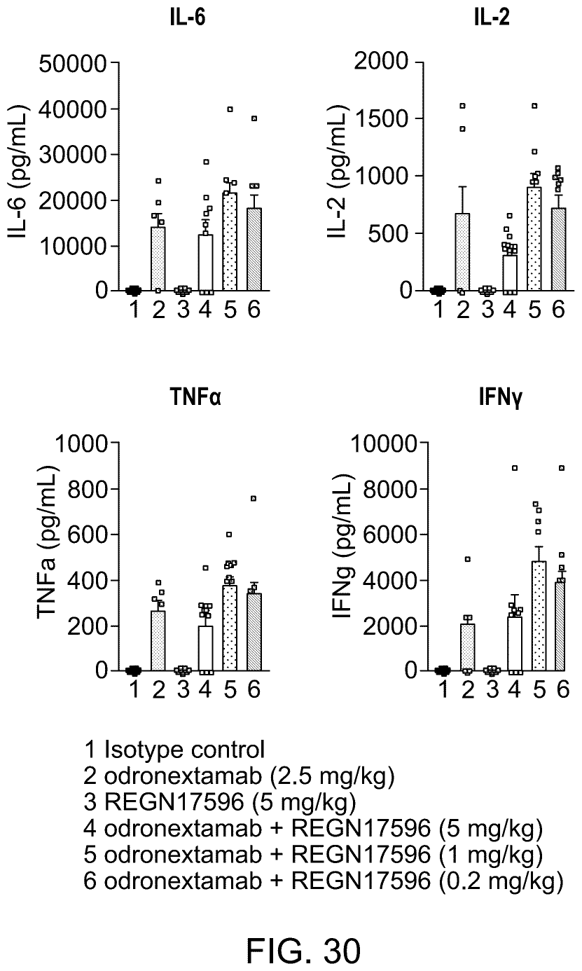

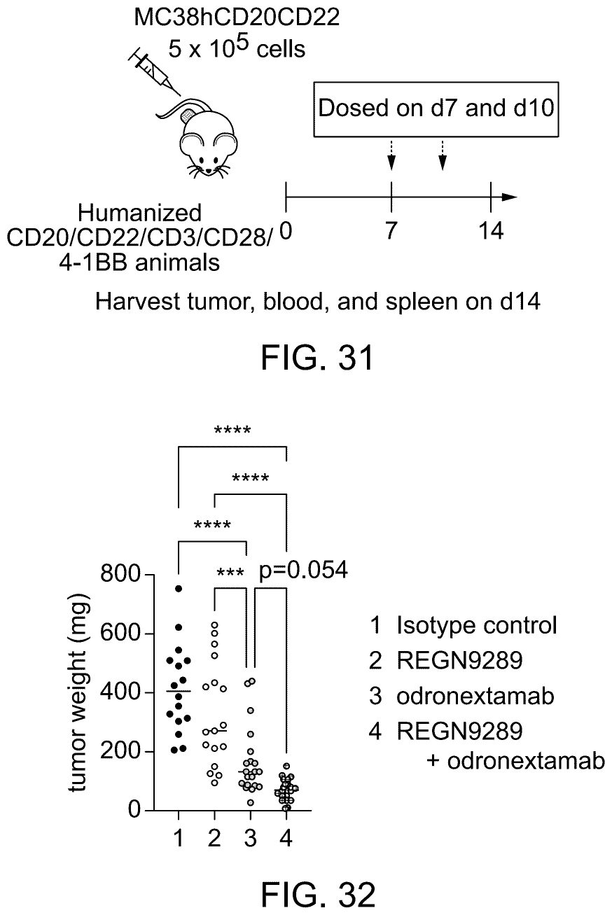

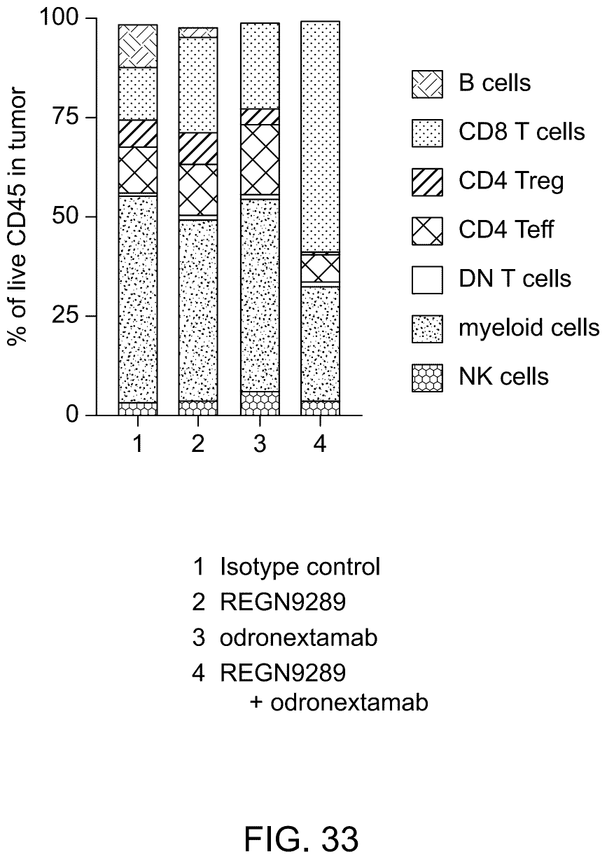

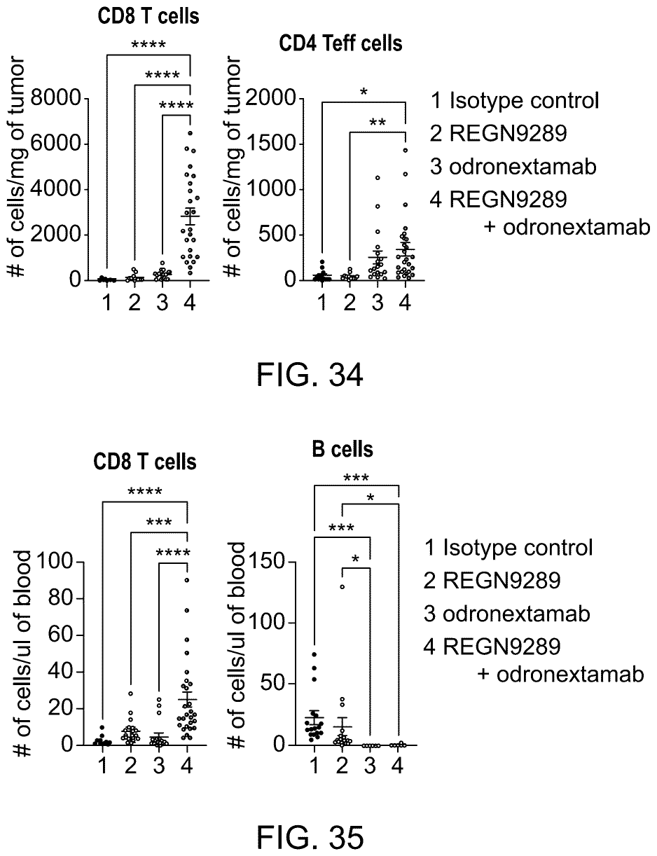

To investigate how the combination of CD22×41BB and odronextamb augments anti-tumor immunity, we evaluated how the different treatments modulated intratumoral T cell responses, 14 days post implantation (FIG. 31). We chose a suboptimal treatment regimen where the combination of odronextamab (2.5 mg/kg) with CD22×41BB (1 mg/kg) significantly enhances survival, but does not mediate maximal anti-tumor immunity to ensure the presence of tumor material to harvest. At this time point, odronextamab treatment significantly decreased tumor mass (odronextamab vs isotype: P<0.0001), and combination treatment seemed to further suppress tumor growth (FIG. 32). High dimensional reduction analysis of intratumoral and splenic immune subsets revealed the enrichment and depletion of certain populations in response to CD3 bispecific or combination treatment. While administration of CD22×41BB preferentially expanded the frequency of intratumoral CD8+ T cells, the addition of odronextamab further significantly increased the proportion of intratumoral CD8 T cells (FIG. 33). The density of T cell subsets was enumerated, and while there was a trend towards an increased density of intratumoral CD8 T cells with odronextamab monotherapy, the combination of CD22×41BB and odronextamab significantly augmented the density of CD8 T cells (combination vs odronextamab: P<0.0001; 2826 CD8 T cells/mg of tumor vs 290 cells/mg of tumor) (FIG. 34). Combination treatment significantly enhanced the intratumoral density of CD4 Teff cells in comparison to isotype and CD22×41BB monotherapy although to a lesser extent than CD8 T cells (isotype vs combination: P<0.05; 54 Teff cells/mg of tumor vs 342 cells/mg of tumor). This significant expansion of CD8 T cells in response to combination treatment is also observed in the blood with a concomitant decrease in B cells (FIG. 35).

### Example 11: In Vivo Efficacy of CD22×4-1BB Antibodies in Combination with Odronextamab in the Xenogeneic Human WSU-DLCL2 (DLBCL) Tumor Model

The ability of CD22×4-1BB bsAbs to augment the anti-tumor activity of odronextamab in vivo was evaluated in the xenogeneic human WSU-DLCL2 (DLBCL) tumor model. On day 0, immunodeficient NSG animals were subcutaneously implanted with WSU-DLCL2 tumor cells mixed with human PBMCs and resuspended in matrigel. Mice were randomized to receive blinded treatments of either non-targeted isotype controls or test articles (REGN9289 [CD22×4-1BB], REGN9220 [CD22×4-1BB], REGN17596 [CD22×4-1BB], or odronextamab [CD20×CD3]) which were administered as monotherapy or in combination by intraperitoneal injection at specified concentrations on day 8, day 15, and day 22 post implantation. These studies demonstrate that while odronextamab (2.5 mg/kg) monotherapy induced modest tumor growth inhibition with enhanced survival, the addition of CD22×4-1BB bsAb to odronextamb induced potent dose-dependent anti-tumor activity with an 88% tumor rejection rate when odronextamab was dosed with REGN9220 at 10 mg/kg, a 63% tumor rejection rate when odronextamab was dosed with REGN9220 at 1 or 0.1 mg/kg and a 25% rejection rate when odronextamab was dosed with REGN9220 at 0.01 mg/kg. The addition of REGN9289 or REGN17596 (at 1 mg/kg) to odronextamab also greatly enhanced anti-tumor immunity with tumor rejection rates of 100% or 75% respectively.

### Experimental Procedure

The WSU-DLCL2 cell line was obtained from DSMZ (ACC 575) and maintained in RPMI-1640 with 10% FBS (Seradigm) supplemented with penicillin, streptomycin, glutamine, and 1 mM HEPES (Gibco). WSU-DLCL2 cells (3×10⁶ cells) were collected and mixed with 5×10⁵ PBMCs (ReachBio) and resuspended in a 1:1 mixture of PBS and GFR Matrigel (Corning). 12-week old female NSG mice were subcutaneously injected with the cell mixture in the right flank. Mice were randomized to receive blinded treatments of either isotype controls or test articles (REGN9220, REGN9289, REGN17596, or odronextamab) which were administered as monotherapy or in combination by intraperitoneal injection at specified concentrations on day 8, day 15, and day 22 post implantation. Tumor growth was monitored over time using digital caliper (VWR) measurements of X and Y diameter (perpendicular measurements of length and width). Tumor volume was calculated (X*Y*(X/2) where X is the shorter diameter). Mice were euthanized when the tumor reached a designated tumor endpoint (tumor diameter >20 mm, or tumor ulceration). This designated endpoint is in accordance with IACUC standards.

### Results Summary and Conclusion

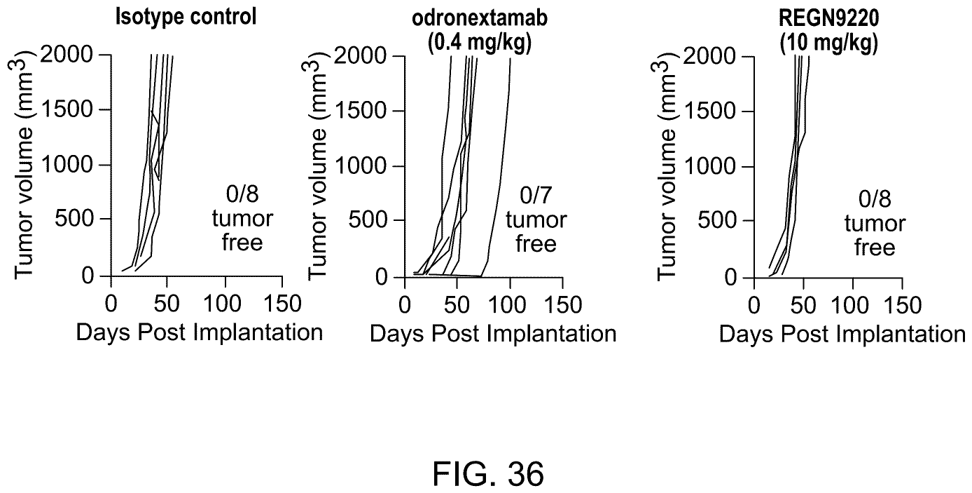

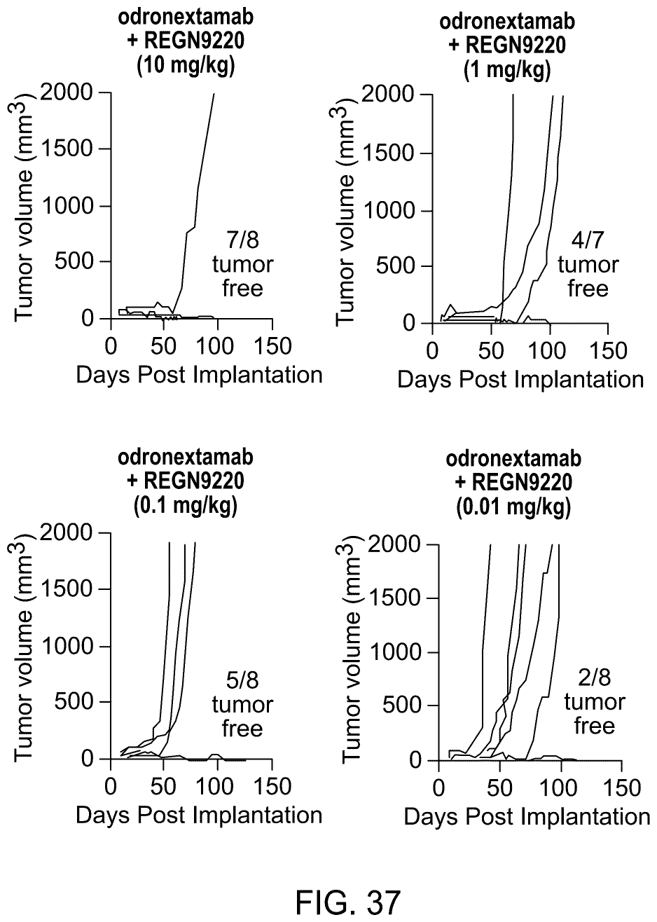

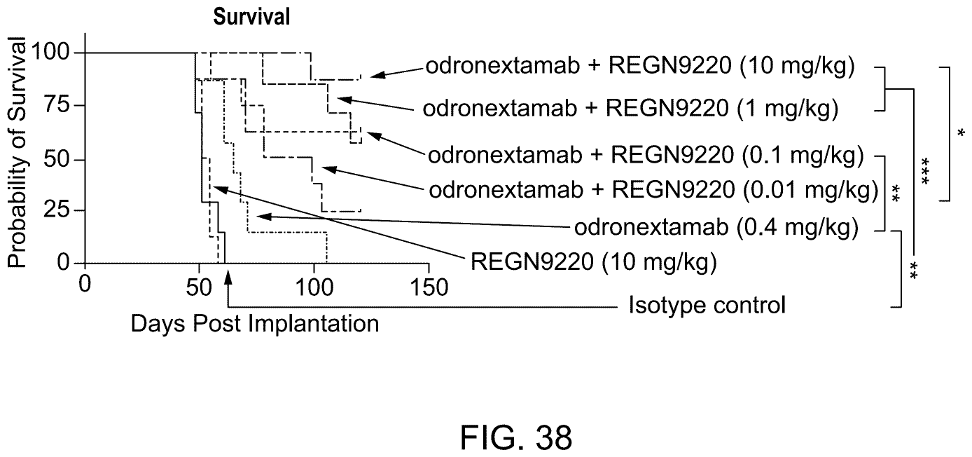

While odronextamab monotherapy treatment was able to delay tumor growth and confer a survival advantage in comparison to isotype control treated animals, all animals in these treatment groups eventually succumbed to tumor outgrowth (FIGS. 36-38). The addition of CD22×4-1BB bsAb further augmented the anti-tumor activity of odronextamb with dose-dependent anti-tumor activity with an 88% tumor rejection rate when odronextamab was dosed with REGN9220 at 10 mg/kg, a 57% tumor rejection rate when odronextamab was dosed with REGN9220 at 1 mg/kg, a 63% tumor rejection rate when odronextamab was dosed with REGN9220 or 0.1 mg/kg, and a 25% rejection rate when odronextamab was dosed with REGN9220 at 0.01 mg/kg. This potent combinatorial activity significantly enhanced survival in comparison to odronextmab activity when REGN9220 was dosed between 0.1 to 10 mg/kg. REGN9289 or REGN17596 (at 1 mg/kg) to odronextamab also greatly enhanced anti-tumor immunity with tumor rejection rates of 100% or 75% respectively. Therefore, these data demonstrate that the synergistic combination of CD22×4-1BB and odronextamab can induce dose-dependent curative anti-tumor activity.

The foregoing merely illustrates the principles of the disclosure. Any examples set forth in this specification are not intended to be limiting and merely set forth some of the many possible embodiments for the appended claims. Those skilled in the art will readily recognize various modifications and changes that may be made without following the example embodiments and applications illustrated and described herein, and without departing from the true spirit and scope of the following claims.

All references cited and/or discussed in this specification are incorporated herein by reference in their entireties and to the same extent as if each reference was individually incorporated by reference.

1. An isolated multispecific antigen-binding molecule comprising:
(a) a first antigen-binding domain (D1) comprising three complementarity determining regions (CDRs) (D1-HCDR1, D1-HCDR2, and D1-HCDR3) of a heavy chain variable region (D1-HCVR) and three CDRs (D1-LCDR1, D1-LCDR2, and D1-LCDR3) of a light chain variable region (D1-LCVR), wherein the first antigen-binding domain binds specifically to CD22;
(b) a second antigen-binding domain (D2) comprising three CDRs (D2-HCDR1, D2-HCDR2, and D2-HCDR3) of a HCVR (D2-HCVR) and three CDRs (D2-LCDR1, D2-LCDR2, and D2-LCDR3) of a LCVR (D2-LCVR), wherein the second antigen-binding domain binds specifically to 4-1BB; and
(c) a third antigen-binding domain (D3) comprising three CDRs (D3-HCDR1, D3-HCDR2, and D3-HCDR3) of a HCVR (D3-HCVR) and three CDRs (D3-LCDR1, D3-LCDR2, and D3-LCDR3) of a LCVR (D3-LCVR), wherein the third antigen-binding domain binds specifically to 4-1BB.

2. The isolated multispecific antigen-binding molecule of claim 1, wherein D2 and D3 bind to the same epitope on 4-1BB.

3. The isolated multispecific antigen-binding molecule of claim 1, wherein D2 and D3 bind to different epitopes on 4-1BB.

4. The isolated multispecific antigen-binding molecule of claim 1, wherein D1 comprises three CDRs of a HCVR comprising an amino acid sequence of SEQ ID NO: 2 or 32.

5. The isolated multispecific antigen-binding molecule of claim 1, wherein D1 comprises three CDRs of a LCVR comprising an amino acid sequence of SEQ ID NO: 18.

6. The isolated multispecific antigen-binding molecule of claim 1, wherein D1-HCDR1, D1-HCDR2, and D1-HCDR3 comprise respective amino acid sequences of SEQ ID NO: 4, SEQ ID NO: 6, and SEQ ID NO: 8; or SEQ ID NO: 34, SEQ ID NO: 36, and SEQ ID NO: 38.

7. The isolated multispecific antigen-binding molecule of claim 1, wherein D1-LCDR1, D1-LCDR2, and D1-LCDR3 comprise respective amino acid sequences of SEQ ID NO: 20, GAS, and SEQ ID NO: 24.

8. The isolated multispecific antigen-binding molecule of claim 4, wherein D1-HCVR comprises an amino acid sequence of SEQ ID NO: 2 or 32.

9. The isolated multispecific antigen-binding molecule of claim 5, wherein D1-LCVR comprises an amino acid sequence of SEQ ID NO: 18.

10. The isolated multispecific antigen-binding molecule of claim 1, wherein D2 comprises three CDRs of a HCVR comprising an amino acid sequence of SEQ ID NO: 10 or 42.

11. The isolated multispecific antigen-binding molecule of claim 1, wherein D2 comprises three CDRs of a LCVR comprising an amino acid sequence of SEQ ID NO: 18.

12. The isolated multispecific antigen-binding molecule of claim 1, wherein D2-HCDR1, D2-HCDR2, and D2-HCDR3 comprise respective amino acid sequences of SEQ ID NO: 12, SEQ ID NO: 14, and SEQ ID NO: 16; or SEQ ID NO: 44, SEQ ID NO: 46, and SEQ ID NO: 48.

13. The isolated multispecific antigen-binding molecule of claim 1, wherein D2-LCDR1, D2-LCDR2, and D2-LCDR3 comprise respective amino acid sequences of SEQ ID NO: 20, GAS, and SEQ ID NO: 24.

14. The isolated multispecific antigen-binding molecule of claim 10, wherein D2-HCVR comprises an amino acid sequence of SEQ ID NO: 10 or 42.

15. The isolated multispecific antigen-binding molecule of claim 11, wherein D2-LCVR comprises an amino acid sequence of SEQ ID NO: 18.

16. The isolated multispecific antigen-binding molecule of claim 1, wherein D3 comprises three CDRs of a HCVR comprising an amino acid sequence of SEQ ID NO: 10 or 42.

17. The isolated multispecific antigen-binding molecule of claim 1, wherein D3 comprises three CDRs of a LCVR comprising an amino acid sequence of SEQ ID NO: 18.

18. The isolated multispecific antigen-binding molecule of claim 1, wherein D3-HCDR1, D3-HCDR2, and D3-HCDR3 comprise respective amino acid sequences of SEQ ID NO: 12, SEQ ID NO: 14, and SEQ ID NO: 16; or SEQ ID NO: 44, SEQ ID NO: 46, and SEQ ID NO: 48.

19. The isolated multispecific antigen-binding molecule of claim 1, wherein D3-LCDR1, D3-LCDR2, and D3-LCDR3 comprise respective amino acid sequences of SEQ ID NO: 20, GAS, and SEQ ID NO: 24.

20. The isolated multispecific antigen-binding molecule of claim 16, wherein D3-HCVR comprises the amino acid sequence of SEQ ID NO: 10 or 42.

21. The isolated multispecific antigen-binding molecule of claim 17, wherein D3-LCVR comprises the amino acid sequence of SEQ ID NO: 18.

22. The isolated multispecific antigen-binding molecule of claim 1, wherein D1-LCVR, D2-LCVR, and D3-LCVR comprise the same amino acid sequence.

23. The isolated multispecific antigen-binding molecule of claim 1, wherein D1 comprises three CDRs of a HCVR comprising an amino acid sequence of SEQ ID NO: 2 or 32; D2 comprises three CDRs of a HCVR comprising an amino acid sequence of SEQ ID NO: 10 or 42; and D3 comprises three CDRs of a HCVR comprising an amino acid sequence of SEQ ID NO: 10 or 42.

24. The isolated multispecific antigen-binding molecule of claim 1, wherein D1 comprises three CDRs of a HCVR comprising an amino acid sequence of SEQ ID NO: 2; D2 comprises three CDRs of a HCVR comprising an amino acid sequence of SEQ ID NO: 10; and D3 comprises three CDRs of a HCVR comprising an amino acid sequence of SEQ ID NO: 10.

25. The isolated multispecific antigen-binding molecule of claim 1, wherein D1 comprises three CDRs of a HCVR comprising an amino acid sequence of SEQ ID NO: 2; D2 comprises three CDRs of a HCVR comprising an amino acid sequence of SEQ ID NO: 42; and D3 comprises three CDRs of a HCVR comprising an amino acid sequence of SEQ ID NO: 42.

26. The isolated multispecific antigen-binding molecule of claim 1, wherein D1 comprises three CDRs of a HCVR comprising an amino acid sequence of SEQ ID NO: 32; D2 comprises three CDRs of a HCVR comprising an amino acid sequence of SEQ ID NO: 10; and D3 comprises three CDRs of a HCVR comprising an amino acid sequence of SEQ ID NO: 10.

27. The isolated multispecific antigen-binding molecule of claim 24, wherein D1-HCVR, D2-HCVR, and D3-HCVR comprise respective amino acid sequences of SEQ ID NOs: 2, 10, and 10.

28. The isolated multispecific antigen-binding molecule of claim 25, wherein D1-HCVR, D2-HCVR, and D3-HCVR comprise respective amino acid sequences of SEQ ID NOs: 2, 42, and 42.

29. The isolated multispecific antigen-binding molecule of claim 26, wherein D1-HCVR, D2-HCVR, and D3-HCVR comprise respective amino acid sequences of SEQ ID NOs: 32, 10, and 10.

30. The isolated multispecific antigen-binding molecule of claim 1, wherein D1-HCVR, D2-HCVR, and D3-HCVR comprise respective amino acid sequences of SEQ ID NOs: 2, 10, and 10; and D1-LCVR, D2-LCVR, and D3-LCVR comprise respective amino acid sequences of SEQ ID NOs: 18, 18, and 18.

31. The isolated multispecific antigen-binding molecule of claim 1, wherein D1-HCVR, D2-HCVR, and D3-HCVR comprise respective amino acid sequences of SEQ ID NOs: 2, 42, and 42; and D1-LCVR, D2-LCVR, and D3-LCVR comprise respective amino acid sequences of SEQ ID NOs: 18, 18, and 18.

32. The isolated multispecific antigen-binding molecule of claim 1, wherein D1-HCVR, D2-HCVR, and D3-HCVR comprise respective amino acid sequences of SEQ ID NOs: 32, 10, and 10; and D1-LCVR, D2-LCVR, and D3-LCVR comprise respective amino acid sequences of SEQ ID NOs: 18, 18, and 18.

33. The isolated multispecific antigen-binding molecule of claim 1, wherein the multispecific antigen-binding protein is a multispecific antibody or antigen-binding fragment thereof.

34. The isolated multispecific antigen-binding molecule of claim 33, wherein the multispecific antigen-binding the molecule is a multispecific antibody comprising a first heavy chain comprising a HCVR of a first antigen-binding arm, which binds CD22, wherein the first heavy chain is paired with a first light chain comprising a LCVR of the first antigen-binding arm, wherein (i) the first antigen-binding arm binds CD22, (ii) the HCVR is D1-HCVR, and (iii) the LCVR is D1-LCVR.

35. The isolated multispecific antigen-binding molecule of claim 34, wherein the multispecific antigen-binding the molecule is a multispecific antibody further comprising a second heavy chain comprising an outer HCVR and an inner HCVR of a second antigen-binding arm, wherein the second heavy chain is paired with an outer light chain and an inner light chain comprising an outer LCVR and an inner LCVR, respectively, of the second antigen-binding arm, wherein (i) the second antigen-binding arm binds 4-1BB, (ii) the outer HCVR is D2-HCVR, (iii) the inner HCVR is D3-HCVR, (iv) the outer LCVR is D2-LCVR, and (vi) the inner LCVR is D3-LCVR.

36. The isolated multispecific antigen-binding molecule of claim 35, wherein the first heavy chain and the first light chain are interconnected by disulfide bonds, wherein the first heavy chain comprises D1-HCVR and a heavy chain constant region comprising CH1, CH2, and CH3 domains, and the first light chain comprises D1-LCVR and a light chain constant region, wherein the first heavy chain and first light chain comprise the first antigen-binding domain; and
the second heavy chain and the outer light chain are interconnected by disulfide bonds, and the second heavy chain and the inner light chain are interconnected by disulfide bonds, wherein the second heavy chain comprises D2-HCVR and D3-HCVR and a heavy chain constant region comprising CH1, CH2, and CH3 domains, and the outer light chain comprises D2-LCVR and a light chain constant region, and the inner light chain comprises D3-LCVR and a light chain constant region, wherein the second heavy chain and the outer light chain comprise the second antigen-binding domain, and the second heavy chain and the inner light chain comprise the third antigen-binding domain.

37. The isolated multispecific antigen-binding molecule of claim 34, wherein the first heavy chain or the second heavy chain, but not both, comprises a CH3 domain comprising a H435R (EU numbering) modification and a Y436F (EU numbering) modification.

38. The isolated multispecific antigen-binding molecule of claim 34, wherein the heavy chain constant region of the first heavy chain and/or the heavy chain constant region of the second heavy chain are of isotype IgG1.

39. The isolated multispecific antigen-binding molecule of claim 34, wherein the heavy chain constant region of the first heavy chain and/or the heavy chain constant region of the second heavy chain are of isotype IgG4.

40. The isolated multispecific antigen-binding molecule of claim 34, wherein the first heavy chain and the second heavy chain comprise a chimeric hinge that reduces Fc receptor binding relative to a wild-type hinge of the same isotype.

41. The isolated multispecific antigen-binding molecule of claim 34, wherein the first heavy chain comprises an amino acid sequence of SEQ ID NO: 26 or 40.

42. The isolated multispecific antigen-binding molecule of claim 34, wherein the second heavy chain comprises an amino acid sequence of SEQ ID NO: 28 or 50.

43. The isolated multispecific antigen-binding molecule of claim 34, wherein the first heavy chain comprises an amino acid sequence of SEQ ID NO: 26, and the second heavy chain comprises an amino acid sequence of SEQ ID NO: 28.

44. The isolated multispecific antigen-binding molecule of claim 34, wherein the first heavy chain comprises an amino acid sequence of SEQ ID NO: 26, and the second heavy chain comprises an amino acid sequence of SEQ ID NO: 50.

45. The isolated multispecific antigen-binding molecule of claim 34, wherein the first heavy chain comprises an amino acid sequence of SEQ ID NO: 40, and the second heavy chain comprises an amino acid sequence of SEQ ID NO: 28.

46. The isolated multispecific antigen-binding molecule of claim 34, wherein the first light chain, the outer light chain, and the inner light chain comprise a common light chain.

47. The isolated multispecific antigen-binding molecule of claim 46, wherein the common light chain comprises an amino acid sequence of SEQ ID NO: 30.

48. A pharmaceutical composition comprising the isolated multispecific antigen-binding molecule of claim 1, and a pharmaceutically acceptable carrier or diluent.

49. A method for making the isolated multispecific antigen-binding molecule of claim 1 comprising: (a) introducing one or more nucleic acid molecules comprising polynucleotide sequences that encode the antigen-binding molecule into a host cell, and (b) culturing the host cell under conditions favorable to expression of the one or more nucleic acid molecules.

50. The method of claim 49, further comprising (c) isolating the multispecific antigen-binding molecule from the host cell and/or medium in which the host cell is grown.

51. The method of claim 50, wherein the host cell is a Chinese hamster ovary (CHO) cell.

52. A nucleic acid molecule or a group of nucleic acid molecules comprising one or more nucleotide sequences, respectively, encoding D1-HCVR, D2-HCVR, D3-HCVR, D1-LCVR, D2-LCVR, and D3-LCVR of claim 1.

53. An expression vector comprising the nucleic acid molecule of claim 52, or a group of expression vectors comprising, respectively, the group of nucleic acid molecules of claim 52.

54. A host cell comprising the expression vector or group of expression vectors of claim 53.

55. A host cell comprising:
(a) a first expression vector comprising a nucleic acid molecule encoding a first immunoglobulin heavy chain comprising the amino acid sequence of SEQ ID NO: 26;
a second expression vector comprising a nucleic acid molecule encoding a second immunoglobulin heavy chain comprising the amino acid sequence of SEQ ID NO: 28; and
a third expression vector comprising a nucleic acid molecule encoding an immunoglobulin light chain comprising the amino acid sequence of SEQ ID NO: 30;
(b) a first expression vector comprising a nucleic acid molecule encoding a first immunoglobulin heavy chain comprising the amino acid sequence of SEQ ID NO: 26;
a second expression vector comprising a nucleic acid molecule encoding a second immunoglobulin heavy chain comprising the amino acid sequence of SEQ ID NO: 50; and
a third expression vector comprising a nucleic acid molecule encoding an immunoglobulin light chain comprising the amino acid sequence of SEQ ID NO: 30; or
(c) a first expression vector comprising a nucleic acid molecule encoding a first immunoglobulin heavy chain comprising the amino acid sequence of SEQ ID NO: 40;
a second expression vector comprising a nucleic acid molecule encoding a second immunoglobulin heavy chain comprising the amino acid sequence of SEQ ID NO: 28; and
a third expression vector comprising a nucleic acid molecule encoding an immunoglobulin light chain comprising the amino acid sequence of SEQ ID NO: 30.

56. (canceled)

57. (canceled)

58. A method of producing a multispecific antigen-binding molecule that specifically binds CD22 and 4-1BB comprising: (a) culturing the host cell of claim 55 under conditions favorable for production of the multispecific antigen-binding molecule; and (b) optionally, isolating the multispecific antigen-binding molecule from the host cell and/or medium in which the host cell is grown.

59. The method of claim 58, wherein the host cell is a CHO cell.

60. The method of claim 58 further comprising formulating the multispecific antigen-binding molecule as a pharmaceutical composition comprising an acceptable carrier.

61. A method of inhibiting growth of a tumor in a subject, comprising administering the isolated multispecific antigen-binding molecule of claim 1 to the subject.

62. The method of claim 61, wherein the tumor is anal cancer, angiosarcoma, basal cell carcinoma, a B cell cancer, bladder cancer, bone cancer, brain cancer, breast cancer, cervical cancer, cholangiocarcinoma, chondrosarcoma, colon cancer, colorectal cancer, cutaneous squamous cell carcinoma, endometrial cancer, esophageal cancer, glioblastoma multiforme, head and neck squamous cell cancer, hepatocellular carcinoma, kidney cancer, liver cancer, leukemia, lung cancer, lymphoma, Merkel cell carcinoma, melanoma, myeloma, non-small cell lung cancer, ovarian cancer, pancreatic cancer, prostate cancer, salivary gland cancer, skin cancer, soft tissue sarcoma, stomach cancer, a T cell cancer, testicular cancer, and uterine cancer.

63. The method of claim 61, further comprising administering a second therapeutic agent or therapeutic regimen.

64. The method of claim 63, wherein the second therapeutic agent or therapeutic regimen comprises an Ang2 inhibitor, a BCMA inhibitor, a bispecific antibody comprising a CD28-binding arm, an anti-CD20×anti-CD3 multispecific antigen-binding molecule, an antibody drug conjugate, a bispecific antibody conjugated to an anti-tumor agent, a cancer vaccine, a CD20 inhibitor, a CD19 inhibitor, a CD27 agonist, a CD28 agonist, CD38 inhibitor, a chemotherapeutic drug, a checkpoint inhibitor, a corticosteroid, a CTLA-4 inhibitor, a cytokine, a DNA alkylator, an EGFR inhibitor, a GITR agonist, a histone deacetylase inhibitor, an IL4 inhibitor, an IL6 inhibitor, an immunocytokine, an immunomodulator, a LAG3 inhibitor, a modified IL2, a modified IL12, a MUC16 inhibitor, an oncolytic virus, a PD-1 inhibitor, a PD-L1 inhibitor, a proteasome inhibitor, radiotherapy, a stem cell transplant, surgery, a T cell comprising a chimeric antigen receptor (CAR-Tcell), a VEGF inhibitor, a 4-1BB activator, or combinations thereof.

65. A kit comprising the isolated multispecific antigen-binding molecule that comprises the isolated multispecific antigen-binding molecule of claim 1 in combination with written instructions for use of a therapeutically effective amount of the isolated multispecific antigen-binding molecule for inhibiting the growth of a tumor in a subject.


SEQUENCE LISTING (58 sequences)

SEQ ID NO: 1 - DNA, 375 bp, synthetic construct
  source 1..375: mol_type = other DNA
gaggtgcagctggtgcagtctggagcagaggtgaaaaagcccggggagtctctgaagatctcctgtaagggttctggatacaactttgccacgtactggattgcctgggtgcgccagatgcccgggaaaggcctggagttgatgggaatcatctatcctggtgactctgagaccacatacaacccgtccttccaaggccaggtcaccatctcagccgacaagtccatcagtaacgcctacctgcagtggagcagcctgaaggcctcggacaccgccatgtattattgtgcgagagtaggaggatattgtagtggtaccagttgccacaactggttcgacccctggggcctgggaaccctggtcaccgtctcctca

SEQ ID NO: 2 - protein, 125 aa, synthetic construct
EVQLVQSGAEVKKPGESLKISCKGSGYNFATYWIAWVRQMPGKGLELMGIIYPGDSETTYNPSFQGQVTISADKSISNAYLQWSSLKASDTAMYYCARVGGYCSGTSCHNWFDPWGLGTLVTVSS

SEQ ID NO: 3 - DNA, 24 bp, synthetic construct
  source 1..24: mol_type = other DNA
ggatacaactttgccacgtactgg

SEQ ID NO: 4 - protein, 8 aa, synthetic construct
GYNFATYW

SEQ ID NO: 5 - DNA, 24 bp, synthetic construct
  source 1..24: mol_type = other DNA
atctatcctggtgactctgagacc

SEQ ID NO: 6 - protein, 8 aa, synthetic construct
IYPGDSET

SEQ ID NO: 7 - DNA, 54 bp, synthetic construct
  source 1..54: mol_type = other DNA
gcgagagtaggaggatattgtagtggtaccagttgccacaactggttcgacccc

SEQ ID NO: 8 - protein, 18 aa, synthetic construct
ARVGGYCSGTSCHNWFDP

SEQ ID NO: 9 - DNA, 369 bp, synthetic construct
  source 1..369: mol_type = other DNA
caggttcagctggtgcagtctggaactgaggtgaagaagcctggggcctcagtgaaggtctcctgcaaggcttctgattacacctttaccgactatggtatcacctgggtgcgacaggcccctggacaagggcttgagtggatgggatggatcagcccttacaatggtaacacagactatgcacagaagttccagggcagagtcaccatgaccgcagacacatccaggaccacagcctacatggagctgaggagcctgagatctgacgacacggccgtgtattactgtgcgagaaagggtcaactcgctctctactactattacggtttggacgtctggggccaagggaccacggtcaccgtctcctca

SEQ ID NO: 10 - protein, 123 aa, synthetic construct
QVQLVQSGTEVKKPGASVKVSCKASDYTFTDYGITWVRQAPGQGLEWMGWISPYNGNTDYAQKFQGRVTMTADTSRTTAYMELRSLRSDDTAVYYCARKGQLALYYYYGLDVWGQGTTVTVSS

SEQ ID NO: 11 - DNA, 24 bp, synthetic construct
  source 1..24: mol_type = other DNA
gattacacctttaccgactatggt

SEQ ID NO: 12 - protein, 8 aa, synthetic construct
DYTFTDYG

SEQ ID NO: 13 - DNA, 24 bp, synthetic construct
  source 1..24: mol_type = other DNA
atcagcccttacaatggtaacaca

SEQ ID NO: 14 - protein, 8 aa, synthetic construct
ISPYNGNT

SEQ ID NO: 15 - DNA, 48 bp, synthetic construct
  source 1..48: mol_type = other DNA
gcgagaaagggtcaactcgctctctactactattacggtttggacgtc

SEQ ID NO: 16 - protein, 16 aa, synthetic construct
ARKGQLALYYYYGLDV

SEQ ID NO: 17 - DNA, 324 bp, synthetic construct
  source 1..324: mol_type = other DNA
gaaatagttttgacacagagtcccggcacactgtcactctctcccggggaaagagccaccttgtcatgtagagcaagtcagtcagtctctagctcttatctcgcctggtaccagcagaagccgggacaggcccctagactgctgatctacggggcaagttccagggccaccggaatccccgaccggttcagtggaagcggaagcggaaccgattttactttgacgatttctagactggagccagaggatttcgccgtttactattgtcaacagtacggaagcagcccgtggacgtttggccagggcacgaaggtagaaatcaag

SEQ ID NO: 18 - protein, 108 aa, synthetic construct
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPWTFGQGTKVEIK

SEQ ID NO: 19 - DNA, 21 bp, synthetic construct
  source 1..21: mol_type = other DNA
cagtcagtctctagctcttat

SEQ ID NO: 20 - protein, 7 aa, synthetic construct
QSVSSSY

SEQ ID NO: 21-22 - skipped (2 entries)

SEQ ID NO: 23 - DNA, 27 bp, synthetic construct
  source 1..27: mol_type = other DNA
caacagtacggaagcagcccgtggacg

SEQ ID NO: 24 - protein, 9 aa, synthetic construct
QQYGSSPWT

SEQ ID NO: 25 - DNA, 1356 bp, synthetic construct
  source 1..1356: mol_type = other DNA
gaggtgcagctggtgcagtctggagcagaggtgaaaaagcccggggagtctctgaagatctcctgtaagggttctggatacaactttgccacgtactggattgcctgggtgcgccagatgcccgggaaaggcctggagttgatgggaatcatctatcctggtgactctgagaccacatacaacccgtccttccaaggccaggtcaccatctcagccgacaagtccatcagtaacgcctacctgcagtggagcagcctgaaggcctcggacaccgccatgtattattgtgcgagagtaggaggatattgtagtggtaccagttgccacaactggttcgacccctggggcctgggaaccctggtcaccgtctcctcagcctccaccaagggcccatcggtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccgtgcccagcaccacctgtggcaggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctcaccgtggacaagagcaggtggcaggaggggaatgtcttctcatgctccgtgatgcatgaggctctgcacaaccactacacacagaagtccctctccctgtctctgggtaaatga

SEQ ID NO: 26 - protein, 451 aa, synthetic construct
EVQLVQSGAEVKKPGESLKISCKGSGYNFATYWIAWVRQMPGKGLELMGIIYPGDSETTYNPSFQGQVTISADKSISNAYLQWSSLKASDTAMYYCARVGGYCSGTSCHNWFDPWGLGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 27 - DNA, 2064 bp, synthetic construct
  source 1..2064: mol_type = other DNA
caggtgcaactcgtgcagtctgggactgaggttaaaaaacctggtgcctccgtcaaagtgtcttgtaaagcatcagattacacttttaccgattatggaatcacatgggttagacaggctcctggacagggattggaatggatgggatggatttctccttacaatggtaacaccgattacgcacaaaaatttcaaggccgcgtgactatgacagcagacacttcccgaactaccgcttacatggaactcaggtccttgcgttctgacgataccgcagtttattattgtgccaggaagggacagttggcactctactattactatggtctggatgtgtgggggcagggcactaccgtcaccgtgagctcagcatctacaaaagggccatccgttttccctctggcaccctgttcaagatctacctctgaatctacagccgctctcggatgcctcgtgaaagactatttcccagaacccgtcaccgtttcatggaacagcggcgccctcacatccggcgtgcatacctttcctgcagtgctccaatcttccggtctgtattctctgtctagtgtcgtgactgttccatcatcttccctcggaactaaaacctatacctgtaacgttgaccataagccctctaacactaaggtggataaaagagtcgaatccaagtacggccctccaggcggcggtggatctggaggcggtggaagccaggttcagctggtgcagtctggaactgaggtgaagaagcctggggcctcagtgaaggtctcctgcaaggcttctgattacacctttaccgactatggtatcacctgggtgcgacaggcccctggacaagggcttgagtggatgggatggatcagcccttacaatggtaacacagactatgcacagaagttccagggcagagtcaccatgaccgcagacacatccaggaccacagcctacatggagctgaggagcctgagatctgacgacacggccgtgtattactgtgcgagaaagggtcaactcgctctctactactattacggtttggacgtctggggccaagggaccacggtcaccgtctcctcagcctctacaaagggaccttctgtgtttcctctggctccttgttctagatctacatctgaatctacagctgctctgggatgtctggtgaaggattattttcctgaacctgtgacagtgtcttggaattctggagctctgacatctggagtgcatacatttcctgctgtgctgcagtcttctggactgtattctctgtcttctgtggtgacagtgccttcttcttctctgggaacaaagacatatacatgtaatgtggatcataagccttctaatacaaaggtggataagagagtggaatctaagtatggacctccttgtcctccttgtcctgctcctcctgtggctggaccttctgtgtttctgtttcctcctaagcctaaggatacactgatgatctctagaacacctgaagtgacatgtgtggtggtggatgtgtctcaggaagatcctgaagtgcagtttaattggtatgtggatggagtggaagtgcataatgctaagacaaagcctagagaagaacagtttaattctacatatagagtggtgtctgtgctgacagtgctgcatcaggattggctgaatggaaaggaatataagtgtaaggtgtctaataagggactgccttcttctatcgaaaagacaatctctaaggctaagggacagcctagagaacctcaggtgtatacactgcctccttctcaggaagaaatgacaaagaatcaggtgtctctgacatgtctggtgaagggattttatccttctgatatcgctgtggaatgggaatctaatggacagcctgaaaataattataagacaacacctcctgtgctggattctgatggatctttttttctgtattctagactgacagtggataagtctagatggcaggaaggaaatgtgttttcttgttctgtgatgcatgaagctctgcataatagatttacacagaagtctctgtctctgtctcctggaaagtag

SEQ ID NO: 28 - protein, 687 aa, synthetic construct
QVQLVQSGTEVKKPGASVKVSCKASDYTFTDYGITWVRQAPGQGLEWMGWISPYNGNTDYAQKFQGRVTMTADTSRTTAYMELRSLRSDDTAVYYCARKGQLALYYYYGLDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPGGGGSGGGGSQVQLVQSGTEVKKPGASVKVSCKASDYTFTDYGITWVRQAPGQGLEWMGWISPYNGNTDYAQKFQGRVTMTADTSRTTAYMELRSLRSDDTAVYYCARKGQLALYYYYGLDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNRFTQKSLSLSPGK

SEQ ID NO: 29 - DNA, 648 bp, synthetic construct
  source 1..648: mol_type = other DNA
gaaatagttttgacacagagtcccggcacactgtcactctctcccggggaaagagccaccttgtcatgtagagcaagtcagtcagtctctagctcttatctcgcctggtaccagcagaagccgggacaggcccctagactgctgatctacggggcaagttccagggccaccggaatccccgaccggttcagtggaagcggaagcggaaccgattttactttgacgatttctagactggagccagaggatttcgccgtttactattgtcaacagtacggaagcagcccgtggacgtttggccagggcacgaaggtagaaatcaagcgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 30 - protein, 215 aa, synthetic construct
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 31 - DNA, 381 bp, synthetic construct
  source 1..381: mol_type = other DNA
gaggtgcagctggtggagtctgggggaggcttggtccagccgggggggtccctgaaactctcctgtgcagcctctgggttcaccttcagtggctctgatatgcactgggtccgccaggcttccgggaaagggctggagtgggttggccgtattagaaaccaacctaatagttacgcgacagcatatgctgcgtcggtgaaaggcaggttcaccatctccagagatgattcaaagaacacggcgcatttgcaaatgaacagcctgaaaaccgaggacacggccgtgtatttttgtactagacaaaagcaggtcgtttataattaccatcactactacggtatggacgtctggggccaagggaccacggtcaccgtctcctca

SEQ ID NO: 32 - protein, 127 aa, synthetic construct
EVQLVESGGGLVQPGGSLKLSCAASGFTFSGSDMHWVRQASGKGLEWVGRIRNQPNSYATAYAASVKGRFTISRDDSKNTAHLQMNSLKTEDTAVYFCTRQKQVVYNYHHYYGMDVWGQGTTVTVSS

SEQ ID NO: 33 - DNA, 24 bp, synthetic construct
  source 1..24: mol_type = other DNA
gggttcaccttcagtggctctgat

SEQ ID NO: 34 - protein, 8 aa, synthetic construct
GFTFSGSD

SEQ ID NO: 35 - DNA, 30 bp, synthetic construct
  source 1..30: mol_type = other DNA
attagaaaccaacctaatagttacgcgaca

SEQ ID NO: 36 - protein, 10 aa, synthetic construct
IRNQPNSYAT

SEQ ID NO: 37 - DNA, 54 bp, synthetic construct
  source 1..54: mol_type = other DNA
actagacaaaagcaggtcgtttataattaccatcactactacggtatggacgtc

SEQ ID NO: 38 - protein, 18 aa, synthetic construct
TRQKQVVYNYHHYYGMDV

SEQ ID NO: 39 - DNA, 1362 bp, synthetic construct
  source 1..1362: mol_type = other DNA
gaggtgcagctggtggagtctgggggaggcttggtccagccgggggggtccctgaaactctcctgtgcagcctctgggttcaccttcagtggctctgatatgcactgggtccgccaggcttccgggaaagggctggagtgggttggccgtattagaaaccaacctaatagttacgcgacagcatatgctgcgtcggtgaaaggcaggttcaccatctccagagatgattcaaagaacacggcgcatttgcaaatgaacagcctgaaaaccgaggacacggccgtgtatttttgtactagacaaaagcaggtcgtttataattaccatcactactacggtatggacgtctggggccaagggaccacggtcaccgtctcctcagcctccaccaagggcccatcggtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccgtgcccagcaccacctgtggcaggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctcaccgtggacaagagcaggtggcaggaggggaatgtcttctcatgctccgtgatgcatgaggctctgcacaaccactacacacagaagtccctctccctgtctctgggtaaatga

SEQ ID NO: 40 - protein, 453 aa, synthetic construct
EVQLVESGGGLVQPGGSLKLSCAASGFTFSGSDMHWVRQASGKGLEWVGRIRNQPNSYATAYAASVKGRFTISRDDSKNTAHLQMNSLKTEDTAVYFCTRQKQVVYNYHHYYGMDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 41 - DNA, 357 bp, synthetic construct
  source 1..357: mol_type = other DNA
caggttcagctggtgcagtctggagctgaggtgaagaagcctggggcctcagtgaaggtctcctgcaaggcttctgcttacacctttaccagctatggtttcagctgggtgcgacaggcccctggacaagggcttgagtggatgggatggatcagcacttccaatggttacacaaactatgcacagaagatccaggacagagtcaccatgaccacagacacatccacgagcacagcctacatggagctgaggagcctgagatctgacgacacggccgtgtattattgtgcgagcctctctaactgggtatctttctttgactactggggccagggaaccctggtcaccgtctcctca

SEQ ID NO: 42 - protein, 119 aa, synthetic construct
QVQLVQSGAEVKKPGASVKVSCKASAYTFTSYGFSWVRQAPGQGLEWMGWISTSNGYTNYAQKIQDRVTMTTDTSTSTAYMELRSLRSDDTAVYYCASLSNWVSFFDYWGQGTLVTVSS

SEQ ID NO: 43 - DNA, 24 bp, synthetic construct
  source 1..24: mol_type = other DNA
gcttacacctttaccagctatggt

SEQ ID NO: 44 - protein, 8 aa, synthetic construct
AYTFTSYG

SEQ ID NO: 45 - DNA, 24 bp, synthetic construct
  source 1..24: mol_type = other DNA
atcagcacttccaatggttacaca

SEQ ID NO: 46 - protein, 8 aa, synthetic construct
ISTSNGYT

SEQ ID NO: 47 - DNA, 36 bp, synthetic construct
  source 1..36: mol_type = other DNA
gcgagcctctctaactgggtatctttctttgactac

SEQ ID NO: 48 - protein, 12 aa, synthetic construct
ASLSNWVSFFDY

SEQ ID NO: 49 - DNA, 2040 bp, synthetic construct
  source 1..2040: mol_type = other DNA
caagtgcaacttgtgcagtcaggggcagaagttaagaaacctggggcctccgttaaagtttcttgtaaagctagtgcatacactttcacatcatacggatttagttgggtaagacaagctcctggtcaaggactggaatggatgggatggatttctacttctaacggatacacaaactacgctcagaaaattcaggatagggtaacaatgacaaccgacacttccacatctaccgcatatatggaactcagatcacttagaagcgacgatacagctgtttattactgcgcctctctctccaactgggtgtcatttttcgactattgggggcaaggtaccttggtaacagttagttctgcatctacaaaagggccatccgttttccctctggcaccctgttcaagatctacctctgaatctacagccgctctcggatgcctcgtgaaagactatttcccagaacccgtcaccgtttcatggaacagcggcgccctcacatccggcgtgcatacctttcctgcagtgctccaatcttccggtctgtattctctgtctagtgtcgtgactgttccatcatcttccctcggaactaaaacctatacctgtaacgttgaccataagccctctaacactaaggtggataaaagagtcgaatccaagtacggccctccaggcggcggtggatctggaggcggtggaagccaggttcagctggtgcagtctggagctgaggtgaagaagcctggggcctcagtgaaggtctcctgcaaggcttctgcttacacctttaccagctatggtttcagctgggtgcgacaggcccctggacaagggcttgagtggatgggatggatcagcacttccaatggttacacaaactatgcacagaagatccaggacagagtcaccatgaccacagacacatccacgagcacagcctacatggagctgaggagcctgagatctgacgacacggccgtgtattattgtgcgagcctctctaactgggtatctttctttgactactggggccagggaaccctggtcaccgtctcctcagcctctacaaagggaccttctgtgtttcctctggctccttgttctagatctacatctgaatctacagctgctctgggatgtctggtgaaggattattttcctgaacctgtgacagtgtcttggaattctggagctctgacatctggagtgcatacatttcctgctgtgctgcagtcttctggactgtattctctgtcttctgtggtgacagtgccttcttcttctctgggaacaaagacatatacatgtaatgtggatcataagccttctaatacaaaggtggataagagagtggaatctaagtatggacctccttgtcctccttgtcctgctcctcctgtggctggaccttctgtgtttctgtttcctcctaagcctaaggatacactgatgatctctagaacacctgaagtgacatgtgtggtggtggatgtgtctcaggaagatcctgaagtgcagtttaattggtatgtggatggagtggaagtgcataatgctaagacaaagcctagagaagaacagtttaattctacatatagagtggtgtctgtgctgacagtgctgcatcaggattggctgaatggaaaggaatataagtgtaaggtgtctaataagggactgccttcttctatcgaaaagacaatctctaaggctaagggacagcctagagaacctcaggtgtatacactgcctccttctcaggaagaaatgacaaagaatcaggtgtctctgacatgtctggtgaagggattttatccttctgatatcgctgtggaatgggaatctaatggacagcctgaaaataattataagacaacacctcctgtgctggattctgatggatctttttttctgtattctagactgacagtggataagtctagatggcaggaaggaaatgtgttttcttgttctgtgatgcatgaagctctgcataatagatttacacagaagtctctgtctctgtctcctggaaagtag

SEQ ID NO: 50 - protein, 679 aa, synthetic construct
QVQLVQSGAEVKKPGASVKVSCKASAYTFTSYGFSWVRQAPGQGLEWMGWISTSNGYTNYAQKIQDRVTMTTDTSTSTAYMELRSLRSDDTAVYYCASLSNWVSFFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPGGGGSGGGGSQVQLVQSGAEVKKPGASVKVSCKASAYTFTSYGFSWVRQAPGQGLEWMGWISTSNGYTNYAQKIQDRVTMTTDTSTSTAYMELRSLRSDDTAVYYCASLSNWVSFFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNRFTQKSLSLSPGK

SEQ ID NO: 51 - DNA, 369 bp, synthetic construct
  source 1..369: mol_type = other DNA
caggtgcaactcgtgcagtctgggactgaggttaaaaaacctggtgcctccgtcaaagtgtcttgtaaagcatcagattacacttttaccgattatggaatcacatgggttagacaggctcctggacagggattggaatggatgggatggatttctccttacaatggtaacaccgattacgcacaaaaatttcaaggccgcgtgactatgacagcagacacttcccgaactaccgcttacatggaactcaggtccttgcgttctgacgataccgcagtttattattgtgccaggaagggacagttggcactctactattactatggtctggatgtgtgggggcagggcactaccgtcaccgtgagctca

SEQ ID NO: 52 - DNA, 24 bp, synthetic construct
  source 1..24: mol_type = other DNA
gattacacttttaccgattatgga

SEQ ID NO: 53 - DNA, 24 bp, synthetic construct
  source 1..24: mol_type = other DNA
atttctccttacaatggtaacacc

SEQ ID NO: 54 - DNA, 48 bp, synthetic construct
  source 1..48: mol_type = other DNA
gccaggaagggacagttggcactctactattactatggtctggatgtg

SEQ ID NO: 55 - DNA, 357 bp, synthetic construct
  source 1..357: mol_type = other DNA
caagtgcaacttgtgcagtcaggggcagaagttaagaaacctggggcctccgttaaagtttcttgtaaagctagtgcatacactttcacatcatacggatttagttgggtaagacaagctcctggtcaaggactggaatggatgggatggatttctacttctaacggatacacaaactacgctcagaaaattcaggatagggtaacaatgacaaccgacacttccacatctaccgcatatatggaactcagatcacttagaagcgacgatacagctgtttattactgcgcctctctctccaactgggtgtcatttttcgactattgggggcaaggtaccttggtaacagttagttct

SEQ ID NO: 56 - DNA, 24 bp, synthetic construct
  source 1..24: mol_type = other DNA
gcatacactttcacatcatacgga

SEQ ID NO: 57 - DNA, 24 bp, synthetic construct
  source 1..24: mol_type = other DNA
atttctacttctaacggatacaca

SEQ ID NO: 58 - DNA, 36 bp, synthetic construct
  source 1..36: mol_type = other DNA
gcctctctctccaactgggtgtcatttttcgactat